(19) United States
(12) Patent Application Publication
(10) Pub. No.: US 2026/0317827 A1
(43) Pub. Date: Oct. 15, 2026

(54) DNA METHYLATION SIGNATURE OF RETINOBLASTOMA

(71) Applicants: THE SABAN RESEARCH INSTITUTE;CHILDREN'S HOSPITAL LOS ANGELES, Los Angeles, CA (US); UNIVERSITY OF SOUTHERN CALIFORNIA, Los Angeles, CA (US)

(72) Inventors: Jesse Berry, Altadena, CA (US); Liya Xu, San Diego, CA (US); Gangning Liang, Rowland Heights, CA (US); Hongtao Li, Monterey Park, CA (US)

(21) Appl. No.: 19/111,253

(22) PCT Filed: Sep. 13, 2023
(86) PCT No.: PCT/US2023/074094

§ 371 Date: Mar. 12, 2025

Related U.S. Application Data
(60) Provisional application No. 63/375,511, filed on Sep. 13, 2022.

(51) Int. Cl. C12Q 1/6886; C12Q 1/48; C12Q 1/6806; C12Q 1/6813; C12Q 1/686; C12Q 1/6869

(52) U.S. Cl. CPC C12Q 1/6886; C12Q 1/48; C12Q 1/6806; C12Q 1/6813; C12Q 1/686; C12Q 1/6869; C12Y 207/07; C12Q 2600/112; C12Q 2600/118; C12Q 2600/154

(57) ABSTRACT

Provided herein is a DNA methylation signature of retinoblastoma and methods of use.

### CROSS-REFERENCE TO RELATED APPLICATIONS

This application claims the benefit of U.S. Provisional Application No. 63/375,511, filed Sep. 13, 2022, the content of which is herein incorporated by reference in its entirety.

### GOVERNMENT SUPPORT

This invention was made with Government support under Grant Nos. R35 CA209859, P30 EY029220, P30 CA014089, K08 CA232344 and 2R01CA137124, awarded by the National Institute of Health (NIH). The Government has certain rights in this invention.

### INCORPORATION BY REFERENCE OF SEQUENCE LISTING

This application contains a sequence listing. It has been submitted electronically as an XML file titled “2771014WO1.xml”. The sequence listing is 8,184 bytes in size and was created on Sep. 13, 2023. It is hereby incorporated by reference in its entirety.

### BACKGROUND

Retinoblastoma (RB) is a childhood cancer that forms in the developing retina of babies and toddlers. Malignant tumors can form in one or both eyes. Without treatment RB is life threatening; even with treatment there is a high chance of losing the eye if the cancer does not respond to therapy. (1,2) Although the majority of RB cases are initiated by biallelic inactivating mutations of the RB1 tumor suppressor gene (3), approximately 13% of non-hereditary RB have RB1 promotor methylation and silencing. (4-6) In addition, epigenetic deregulation of tumor-promoting pathways has been shown to play a role in RB tumorigenesis and disease progression beyond RB1 inactivation. (7-9)

### SUMMARY OF THE INVENTION

Retinoblastoma (RB) is a cancer that forms in the developing retina of babies and toddlers. The goal of therapy is to cure the tumor, save the eye and maximize vision. However, it is difficult to predict which eyes are likely to respond to therapy. Predictive molecular biomarkers are needed to guide prognosis and optimize treatment decisions. Direct tumor biopsy is not an option for this cancer; however, the aqueous humor (AH) is an alternate source of tumor-derived cell-free DNA (cfDNA). Here it is shown that DNA methylation profiling of the AH is a valid method to identify the methylation status of RB tumors. 294 genes were identified that were directly regulated by methylation that are implicated in p53 tumor suppressor (RB1, p53, p21, and p16) and oncogenic (E2F) pathways. Finally, AH was used to characterize molecular subtypes that can potentially be used to predict the likelihood of treatment success for retinoblastoma patients.

One aspect provides a method to diagnose retinoblastoma (RB) in a subject comprising providing an aqueous humor (AH) sample from said subject; measuring methylation levels of cell-free DNA (cfDNA) from said AH sample; and determining likelihood of RB if at least one gene in said AH sample of cfDNA is hypermethylated or hypomethylated compared to that of a control. In one aspect, the subject is a mammal, such as a human. In one aspect, the control is AH from an unaffected subject or normal control sample. In one aspect, the methylation level of a promoter region of at least one gene is measured and/or a gene body of the at least one gene is measured. In one aspect, the at least one gene is one provided in FIG. 10. In another aspect, the methylation of at least 2, 3, 4, 5, 6, 7, 8, 9, 10 or more genes is determined. In one aspect, the methylation of status of all genes provided in FIG. 10 is determined. One aspect further comprises treating said subject for RB. In one embodiment, the treatment is cryotherapy, thermotherapy, chemotherapy, radiation therapy, internal radiation therapy, high-dose chemotherapy with stem cell rescue, surgery, targeted therapy, those therapies listed in FIG. 10 or a combination thereof. One aspect further provides the correlating the treatment with the altered methylation state of the gene, wherein the gene and correlated treatment are provided in FIG. 10.

One aspect provides a method to predict severity of retinoblastoma (RB) in a subject comprising providing an aqueous humor (AH) sample from said subject; detecting methylation of cell-free DNA (cfDNA) from said AH sample; and determining severity of RB if a plurality of genes in said AH sample is hypermethylated or hypomethylated compared to that of a control. In one aspect, the subject is a mammal, such as a human. In some aspects, the control is AH from a subject without RB. In one aspect, the methylation status of at least 2, 3, 4, 5, 6, 7, 8, 9, 10 or more genes is determined. In one aspect, the plurality of genes is selected from the genes listed in FIG. 8. In aspect, the severe or more aggressive RB is predicted based on altered methylation status of the plurality of genes identified in FIG. 8 by Type I or Type II probes that show altered methylation status of genes that identified with Cluster B of FIG. 7. One aspect further comprises treating said subject for RB, such as with cryotherapy, thermotherapy, chemotherapy, radiation therapy, internal radiation therapy, high-dose chemotherapy with stem cell rescue, surgery (e.g., enucleation), targeted therapy, or a combination thereof.

In one aspect, the methylation levels are determined by sodium bisulfite pyrosequencing, methylation-sensitive single nucleotide primer extension (Ms-SNuPE) reaction, methylation-specific PCR or microarray analysis. In another aspect, the DNA methylation levels are measured after s odium bisulfite modification and analyzed using microarray analysis.

One aspect provides a method to monitor the progress of a patient on treatment for retinoblastoma (RB) comprising: a) measuring DNA methylation levels of a panel of biomarker loci of cell free DNA (cfDNA) in an aqueous humor (AH) sample from said patient for at least two time points during a course of treatment, and b) identifying a change in DNA methylation levels of the from the panel in a), wherein the change in the methylation status identified in b) towards normal levels indicates the treatment is therapeutically efficacious for the patient, wherein normal levels are determined by comparison to a suitable control sample. In one aspect, the change in methylation levels is in one or more genes provided in FIG. 10. In some aspects, the control sample/normal levels is derived from an unaffected individual or normal control sample. In one aspect, the methylation of cfDNA from AH is measured after initial chemotherapy to treat RB. In another aspect, the methylation of cfDNA from AH is measure at the time or following a tumor recurrence. In one aspect, the period of time begins before a therapeutic treatment and concludes after a therapeutic treatment. In one aspect, the period of time begins and concludes after a therapeutic treatment. In one aspect, the period of time begins after a first therapeutic treatment and before a second therapeutic treatment and concludes after a second therapeutic treatment.

One aspect provides a method to determine modified expression of level of one or more genes in a retinoblastoma (RB) patient comprising providing an aqueous humor (AH) sample from said patient; measuring methylation levels of cell-free DNA (cfDNA) from said AH sample; and determining increased or decreased modified expression of said one or more genes if said or more genes in said AH sample of cfDNA is hypermethylated or hypomethylated compared to that of a control. In one aspect, the gene promoter is hypermethylated or hypomethylated compared to that of a control. In one aspect, the gene body is hypermethylated or hypomethylated compared to that of a control.

One aspect provides a kit for detecting gene methylation of cell-free DNA (cfDNA) in aqueous humor (AH) comprising probes or primers specific for one or more of the genes listed in FIG. 10.

### BRIEF DESCRIPTION OF THE DRAWINGS

Exemplary embodiments are illustrated in referenced figures. It is intended that the embodiments and figures disclosed herein are to be considered illustrative rather than restrictive.

FIG. 1A-1C. DNA methylation signatures in retinoblastoma (RB). A. Unsupervised hierarchical clustering of the differentially methylated probes between RB and retina samples. The top annotation indicates sample type. Green and blue bars indicate the retina (green, n=12) and RB samples (RB_SR, n=30), respectively, from dataset GSE57362. Orange bars highlight the four primary RB tumor samples collected for this study (RB_CHLA, n=4). B. MDS plot showing that RB samples (n=34) can be distinguished from retina (n=12) using the panel of differentially methylated probes in A. C. Genomic location of the hyper- and hypo-CpGs on promoter (TSS, 5′UTR, and 1^{st} exon), gene body, other and enhancer. Total means the probe distribution on EPIC array. Source data are provided as a Source Data file.

FIG. 2A-2D. DNA methylation profiles in AH cfDNAs are highly concordant with paired primary RB tumors. A. Violin plot showing the global DNA methylation level of sonicated DNA from the HCT116 cell line. DNA input amounts for EPIC methylation array assays are shown at the bottom. Non-sonicated bulk DNA (200 ng) was used as control. The median methylation values are shown in dots for each sample. B. Smooth Kernel scatter plots showing the DNA methylation patterns of different amounts of sonicated DNA input DNA (y-axis) compared to the bulk DNA sample (x-axis). The r2 values are displayed for each plot. C. MDS plot indicating the concordance of individual CHLA RB primary tumor sample (RB_CHLA, n=4) and the paired AH cfDNA sample (AH_CHLA, n=4) demonstrating AH is a valid substrate to assay tumor methylation signature. Top 10,000 most variably methylated probes were used to generate the plot. D. Unsupervised hierarchical clustering analysis and heatmap representation of the methylation of the probes used in panel C.

FIG. 3A-3C. RB specific DNA methylation profiles are highly correlated in AH and primary tumors. A. Unsupervised hierarchical clustering of differential DNA methylation between retina (n=12) and RB samples including AH cfDNA (n=15) and RB tumor samples (n=34). The probes identified in FIG. 1A were used. B. MDS plot showing AH cfDNA samples (n=15) are clustered with primary RB tumor samples (n=34) and separated from retina (n=12). C. Promoter DNA methylation RB1, MYCN, and SYK genes in normal retina and RB samples. The RB1 germ line mutation status for each sample is shown on the top. “+” indicates RB1 mutation, “−” indicates no RB1 mutation, and “N” indicates that the genotype was not determined. This demonstrates that the AH can be used to identify cases of RB driven by RB1 hypermethylation (CHLA_3, AH and Tumor); additionally, SYC and MYCN promotors are hypomethylated as compared to normal retina suggesting gene activation in RB tumors versus normal retina. Source data are provided as a Source Data file.

FIG. 4A-4E. Pathway analyses of DNA methylation genes in RB. A. Significantly differential DNA methylation in RB versus retina. Each dot represents one probe. −log₁₀(pvalue) for each probe were plotted on the y-axis while the β value difference between RB tumors and normal retina were plotted on the x-axis. The P value was calculated using two-sided Welch's t-tests. The β value change cutoffs of +/−0.3 were shown. Probe locations were shown in red for promoter, blue for gene body and grey for other location. B. Genes regulated by DNA methylation in RB versus normal retina. DNA methylation changes were plotted on the x-axis (Δβ>0.3 or <−0.3). Gene expression changes were plotted on the y-axis (Log 2FC>1 or <−1). Only the probes located in promoter (red) and gene body (blue) were plotted. C. Graphical summary of the IPA pathway analysis of the genes identified in B shows enrichment of several tumor-associated pathways. Source data are provided as a Source Data file. D. The estrogen-mediated S-phase Entry pathway is activated by the upregulation of many pathway components (shown in red) identified in B. E. Promoter DNA hypomethylation of the genes highlighted in D.

FIG. 5A-5D. Identification of specific DNA methylation clusters from AH cfDNAs. A. Unsupervised hierarchical clustering of the 1092 differentially methylated probes between salvaged (AH_SV, dark green on top, n=4) and enucleated (AH_PE, primary enucleated (n=4) and AH_SE, secondary enucleated (n=4), grey and magenta on top) samples (top panel). The bottom panel showed the salvaged and enucleated samples can be well separated by the selected probes. B. Unsupervised hierarchical clustering heatmap of CHLA AH samples (n=12) and enucleated RB tumors (n=30) from GSE57362 cohort (RB_SR, blue) using the 1092 probes in A. C. Unsupervised hierarchical clustering heatmap of the CHLA AH samples (n=12) and enucleated RB tumors (n=67) from GSE58783 cohort (RB_NC, aqua) using the 1092 probes in A. D. Identification of Cluster A (less aggressive) and Cluster B (more aggressive) by the 1092 probes in A. (a) Unsupervised clustering of the 12 CHLA AH samples and 97 enucleated RB tumors (from 868 B and C). (b) MDS plot of all the 109 (12+30+67) samples. (c) MDS plot of the Cluster A (green) and Cluster B (red) samples.

FIG. 6A-6D. The DNA methylation signature for RB treatment outcome prediction. A. The selected panel of 320 differentially methylated probes between Clusters A and B in FIG. 5D can separate Subtype 1 (blue) and Subtype 2 (orange) RB samples from the GSE58783 cohort by Liu et al.36, By comparison, Clusters A and B are identified by green and red bars, respectively. B. Venn diagrams showing the overlap of Cluster A (green) with Subtype 1 tumors (blue) and Cluster B (red) with Subtype 2 (orange) tumors. C and D. Boxplots showing candidate gene DNA hypo-(C) and hyper-methylation (D) in Cluster B (red) RBs compared to Cluster A (dark green) tumors. The DNA methylation status of each gene in normal retina is shown in light green. For each box plot, the line inside the box denotes the median value, the box denotes the Q3 (top boundary) and Q1 (bottom boundary), and the whiskers denotes the maximum (top) and minimum (bottom) respectively. The P value was calculated using two-sided Welch's t-tests. Source data are provided as a Source Data file.

FIG. 7 (corresponds to FIG. 5A). Unsupervised hierarchical clustering of the 1092 differentially methylated probes between salvaged (AH_SV, dark green on top, n=4) and enucleated (AH_PE, primary enucleated (n=4) and AH_SE, secondary enucleated (n=4), grey and magenta on top) samples (top panel). The bottom panel showed the salvaged and enucleated samples can be well separated by the selected probes. The type I probes (288) are unmethylated (shown in green in the upper panel of the heatmap) in Cluster A (salvaged eyes and good patient outcome) and methylated (shown in red in the upper panel of the heatmap) in Cluster B (enucleated eyes and bad outcome); the type II probes (804) are methylated (shown in red in the lower panel of the heatmap) in Cluster A (salvaged eyes and good outcome) and unmethylated (shown in green in the low panel of the heatmap) in Cluster B (enucleated eyes and bad outcome).

FIG. 8 (corresponds to FIGS. 5A and 7). There are 288 type 1 probes which are hyper methylated in cluster A and hypomethylated in cluster B. There are 804 type II probes which are reverse—i.e., HYPOmethylated in A and HYPERmethylated in B. The loci tested for the methylation, as well as the gene, in addition to other information, is provided.

FIG. 9 (corresponds to FIG. 4B). Genes regulated by DNA methylation in RB versus normal retina. DNA methylation changes were plotted on the x-axis (Δβ>0.3 or <−0.3). Gene expression changes were plotted on the y-axis (Log 2FC>1 or <−1). Only the probes located in promoter (red) and gene body (blue) were plotted. The genes showing the name are therapeutic targets of known drugs based on Ingenuity Pathway Analysis (IPA) from QIAGEN.

Through data analysis, 294 markers in DNA were discovered that are linked to gene behavior, and knowing these can predict how genes work. Notably, 45 targets were identified for new medicines and current medicines available to an art worker/physician, presenting an opportunity for more effective treatments against retinoblastoma.

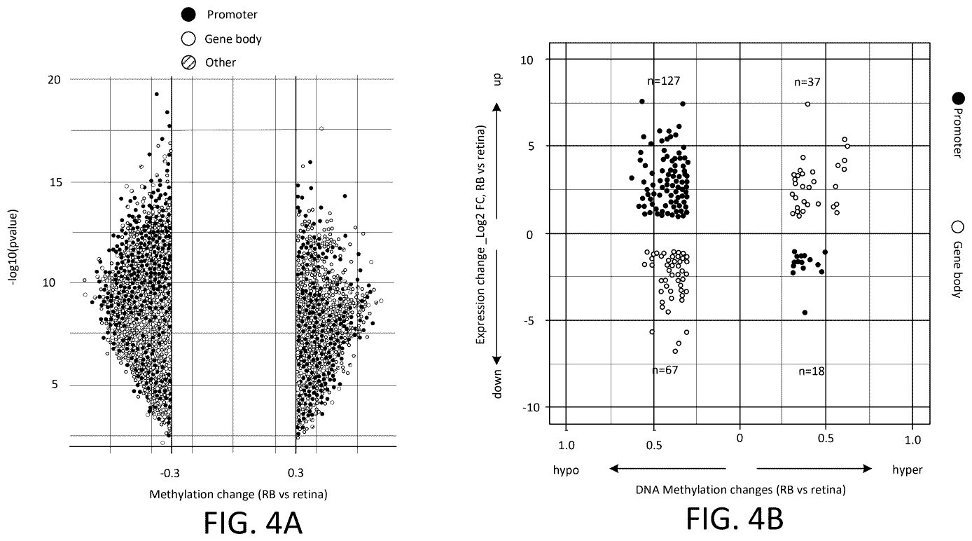

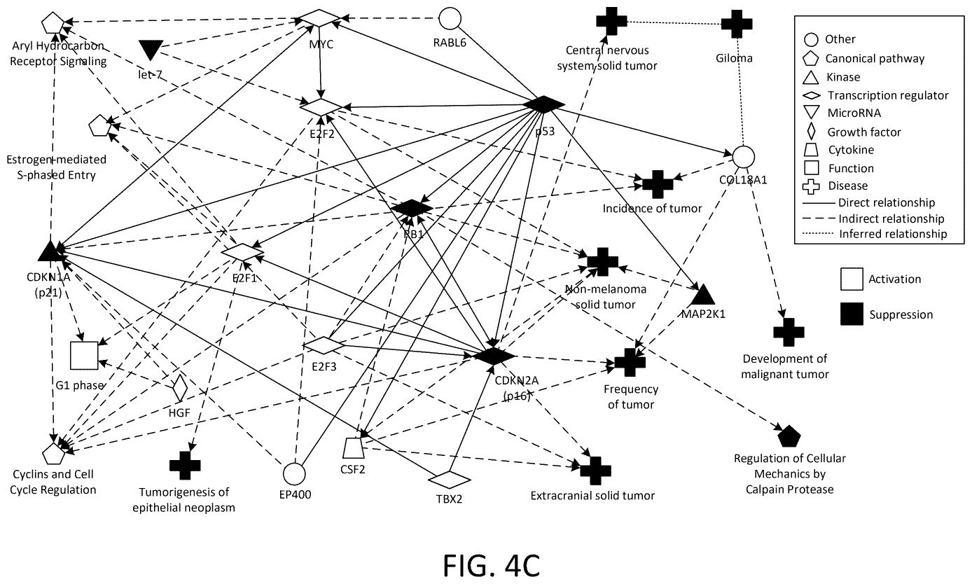

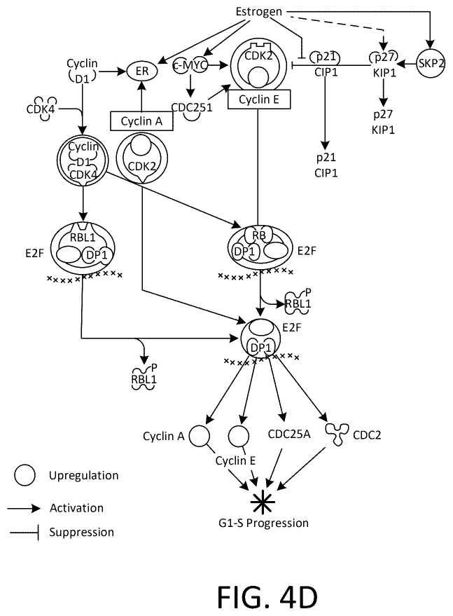

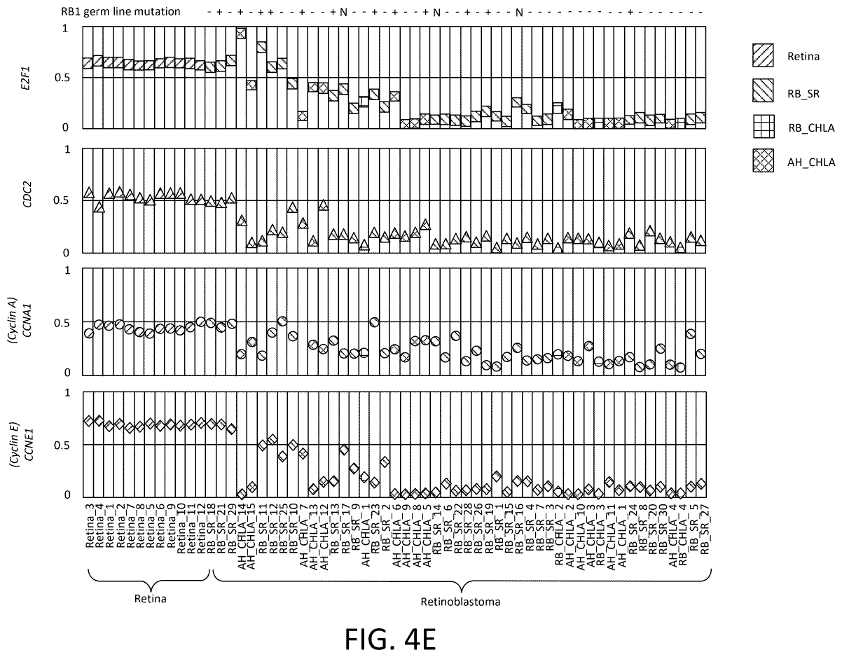

By further analysis of the data in FIG. 4B, it was found that the previously identified 294 DNA methylation markers which were demonstrated highly correlated with gene expression, indeed, by knowing the DNA methylation status in 294 loci, one can also predication genes expression. Furthermore, 45 genes (45 out of 294) were identified as therapeutic targets with candidate drugs by running Ingenuity Pathway Analysis (IPA) from QIAGEN (FIG. 10). In addition, this finding provides us an opportunity to identify new or repurposed drug for the treatment of RB.

FIG. 10 provides the 45 genes as therapeutic targets with candidate drugs for treatment.

FIG. 11 provides the 294 genes/markers in DNA.

FIG. S1A-S1B. Datasets and analysis outline.

FIG. S2A-S2C. LUMP assay filtering the samples purity on the RB_CHLA (orange) and AH_CHLA (red) for this study (A), retina (green) and RB (blue) from GSE57362 (B), and RB (light blue) from GSE58783 (C). Source data are provided as a Source Data file.

FIG. S3A-S3B. Chromosomal Copy number variation (CNV) profiles from 4 eyes with AH cfDNA and corresponding paired primary tumor DNA. (A) SCNA profiles between AH and tumor pairs demonstrate the similar genomic alterations, (B) Salvaged eyes (green, n=4) had fewer copy number alterations than non-Salvaged (enucleated) eyes (blue, n=8). The gain of 1q, 2p, 6p, 7q, and 19p and loss of 13q and 16p are frequently appeared in RB.

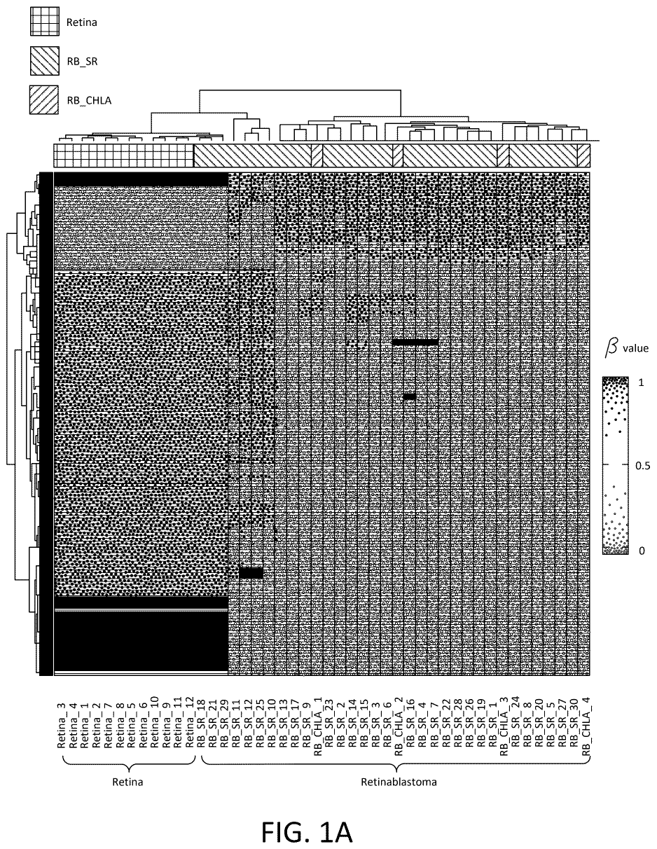

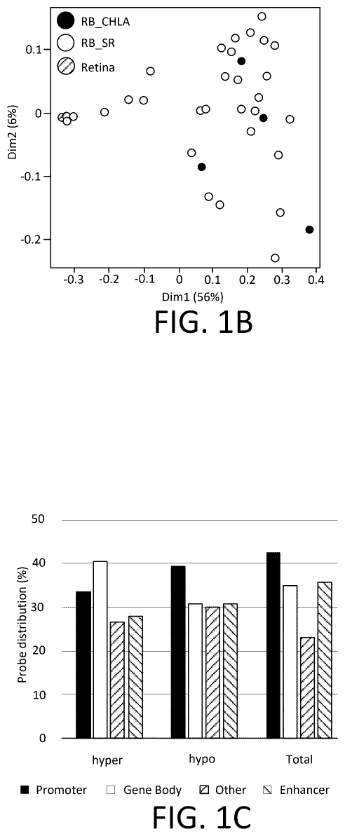

FIG. S4A-S4B. Unsupervised hierarchical clustering (A) and MDS plot (B) showing the RB-specific DNA methylation profile identified in FIG. 1A cannot be detected in RB patient blood samples. Sample types: AH_CHLA (red) for RB AH samples for this study, RB_CHLA (orange) for RB primary tumor samples for this study, RB_SR (blue) for RB samples from GSE57362, Retina (green) for normal retina samples from GSE57362, Blood_CHLA (brown) for RB patient blood plasma samples in this study, and Blood_SR (pink) for RB patient white blood cell samples from GSE57362.

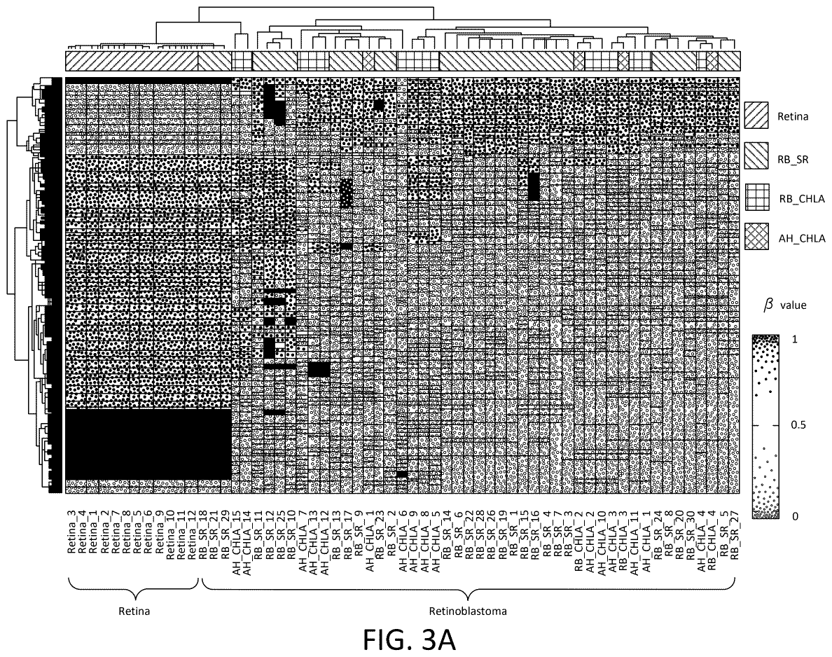

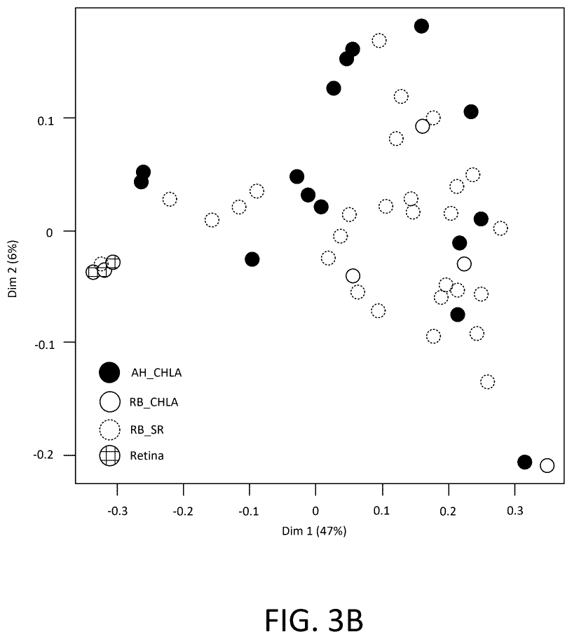

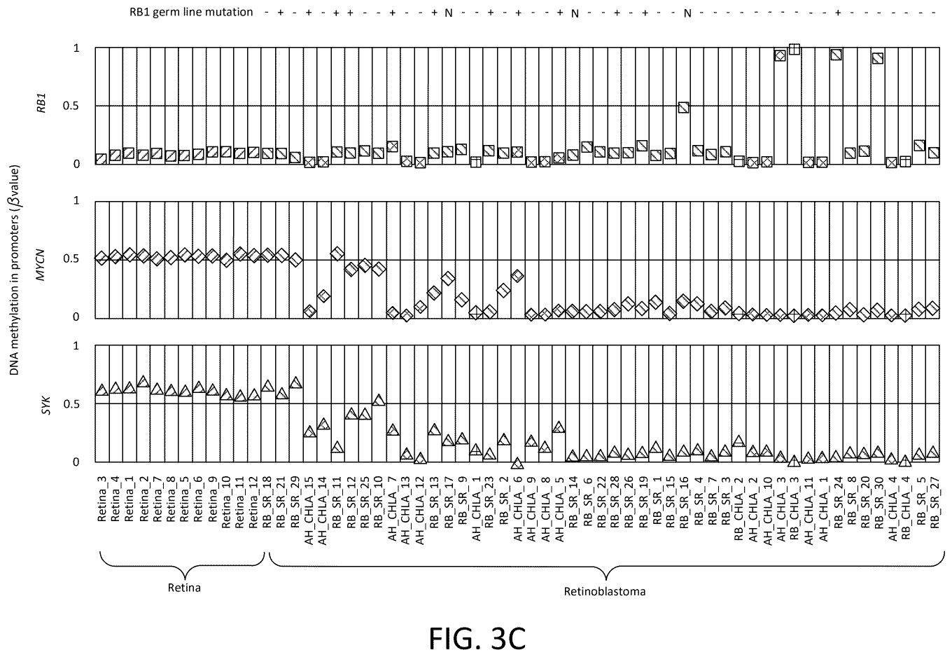

FIG. S5A-S5D. DNA methylation measured by Illumina EPIC array and bisulfite targeted sequencing at the TFF1 promoter (A), the HOXC4 promoter (B), the MNX1 gene body (C) and the CELSR3 gene body (D). a) The mean DNA methylation level (βvalue) of retina and RB specimens from FIG. 3A. For DNA methylation array probes at all four gene loci, the horizontal arrows indicate the transcriptional start sites; b) the mean DNA methylation status using targeted bisulfite sequencing (based on over 100 DNA molecules) for RB (n=10) and retinal (n=1) specimens. Vertical arrows indicate the DNA methylation probe site from DNA methylation array; c) correlation of mean DNA methylation value between array (βvalue) and targeted sequencing (% methylation) for 10 RB DNA specimens. The regression lines were calculated using ggplot2 package with method “lm”. Source data are provided as a Source Data file.

FIG. S6. Top over-represented transcription factor binding sites for methylation regulated genes as determined by TRANSFAC analysis. The P values were calculated by TRANSFAC with default setting.

### DESCRIPTION OF THE INVENTION

### I. Definitions

All references cited herein are incorporated by reference in their entirety as though fully set forth. Unless defined otherwise, technical and scientific terms used herein have the same meaning as commonly understood by one of ordinary skill in the art to which this invention belongs. Singleton et al., Dictionary of Microbiology and Molecular Biology 3^{rd} ed., Revised, J. Wiley & Sons (New York, NY 2006); March, Advanced Organic Chemistry Reactions, Mechanisms and Structure 7^{th} ed., J. Wiley & Sons (New York, NY 2013); and Sambrook and Russel, Molecular Cloning: A Laboratory Manual 4^{th} ed., Cold Spring Harbor Laboratory Press (Cold Spring Harbor, NY 2012), provide one skilled in the art with a general guide to many of the terms used in the present application.

References in the specification to “one embodiment,” “an embodiment,” etc., indicate that the embodiment described may include a particular aspect, feature, structure, moiety, or characteristic, but not every embodiment necessarily includes that aspect, feature, structure, moiety, or characteristic. Moreover, such phrases may, but do not necessarily, refer to the same embodiment referred to in other portions of the specification. Further, when a particular aspect, feature, structure, moiety, or characteristic is described in connection with an embodiment, it is within the knowledge of one skilled in the art to affect or connect such aspect, feature, structure, moiety, or characteristic with other embodiments, whether or not explicitly described.

The singular forms “a,” “an,” and “the” include plural reference unless the context clearly dictates otherwise. Thus, for example, a reference to “a compound” includes a plurality of such compounds, so that a compound X includes a plurality of compounds X. It is further noted that the claims may be drafted to exclude any optional element. As such, this statement is intended to serve as antecedent basis for the use of exclusive terminology, such as “solely,” “only,” and the like, in connection with any element described herein, and/or the recitation of claim elements or use of “negative” limitations. “Plurality” means at least two.

The term “and/or” means any one of the items, any combination of the items, or all of the items with which this term is associated. The phrase “one or more” is readily understood by one of skill in the art, particularly when read in context of its usage. For example, one or more substituents on a phenyl ring refers to one to five, or one to four, for example if the phenyl ring is di-substituted.

As used herein, “or” should be understood to have the same meaning as “and/or” as defined above. For example, when separating a listing of items, “and/or” or “or” shall be interpreted as being inclusive, e.g., the inclusion of at least one, but also including more than one of a number of items, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of,” or “exactly one of.”

As used herein, the terms “including,” “includes,” “having,” “has,” “with,” or variants thereof, are intended to be inclusive similar to the term “comprising.” As used herein the term “comprising” or “comprises” is used in reference to compositions, methods, and respective component(s) thereof, that are useful to an embodiment, yet open to the inclusion of unspecified elements, whether useful or not. It will be understood by those within the art that, in general, terms used herein are generally intended as “open” terms (e.g., the term “including” should be interpreted as “including but not limited to,” the term “having” should be interpreted as “having at least,” the term “includes” should be interpreted as “includes but is not limited to,” etc.). Although the open-ended term “comprising,” as a synonym of terms such as including, containing, or having, is used herein to describe and claim the invention, the present invention, or embodiments thereof, may alternatively be described using alternative terms such as “consisting of” or “consisting essentially of.”

The term “about” can refer to a variation of +5%, +10%, +20%, or +25% of the value specified. For example, “about 50” percent can in some embodiments carry a variation from 45 to 55 percent. For integer ranges, the term “about” can include one or two integers greater than and/or less than a recited integer at each end of the range. Unless indicated otherwise herein, the term “about” is intended to include values, e.g., weight percentages, proximate to the recited range that are equivalent in terms of the functionality of the individual ingredient, the composition, or the embodiment. The term about can also modify the endpoints of a recited range as discuss above in this paragraph. Numerical ranges recited herein by endpoints include all numbers and fractions subsumed within that range (e.g., 1 to 5 includes 1, 1.5, 2, 2.75, 3, 3.90, 4, and 5). It is also to be understood that all numbers and fractions thereof are presumed to be modified by the term “about.”

As will be understood by the skilled artisan, all numbers, including those expressing quantities of ingredients, properties such as molecular weight, reaction conditions, and so forth, are approximations and are understood as being optionally modified in all instances by the term “about.” These values can vary depending upon the desired properties sought to be obtained by those skilled in the art utilizing the teachings of the descriptions herein. It is also understood that such values inherently contain variability necessarily resulting from the standard deviations found in their respective testing measurements.

As will be understood by one skilled in the art, for any and all purposes, particularly in terms of providing a written description, all ranges recited herein also encompass any and all possible sub-ranges and combinations of sub-ranges thereof, as well as the individual values making up the range, particularly integer values. A recited range (e.g., weight percentages or carbon groups) includes each specific value, integer, decimal, or identity within the range. Any listed range can be easily recognized as sufficiently describing and enabling the same range being broken down into at least equal halves, thirds, quarters, fifths, or tenths. As a non-limiting example, each range discussed herein can be readily broken down into a lower third, middle third and upper third, etc. As will also be understood by one skilled in the art, all language such as “up to,” “at least,” “greater than,” “less than,” “more than,” “or more,” and the like, include the number recited and such terms refer to ranges that can be subsequently broken down into sub-ranges as discussed above. In the same manner, all ratios recited herein also include all sub-ratios falling within the broader ratio. Accordingly, specific values recited for radicals, substituents, and ranges, are for illustration only; they do not exclude other defined values or other values within defined ranges for radicals and substituents.

One skilled in the art will also readily recognize that where members are grouped together in a common manner, such as in a Markush group, the invention encompasses not only the entire group listed as a whole, but each member of the group individually and all possible subgroups of the main group.

Additionally, for all purposes, the invention encompasses not only the main group, but also the main group absent one or more of the group members. The invention therefore envisages the explicit exclusion of any one or more of members of a recited group. Accordingly, provisos may apply to any of the disclosed categories or embodiments whereby any one or more of the recited elements, species, or embodiments, may be excluded from such categories or embodiments, for example, for use in an explicit negative limitation.

As used herein, the term “comparing” refers to making an assessment of how the methylation status, proportion, level or cellular localization of one or more biomarkers in a sample from a patient relates to the methylation status, proportion, level or cellular localization of the corresponding one or more biomarkers in a standard or control sample. For example, “comparing” may refer to assessing whether the methylation status, proportion, level, or cellular localization of one or more biomarkers in a sample from a patient is the same as, more or less than, or different from the methylation status, proportion, level, or cellular localization of the corresponding one or more biomarkers in standard or control sample. More specifically, the term may refer to assessing whether the methylation status, proportion, level, or cellular localization of one or more biomarkers in a sample from a patient is the same as, more or less than, different from or otherwise corresponds (or not) to the methylation status, proportion, level, or cellular localization of predefined biomarker levels that correspond to, for example, a patient having retinoblastoma (RB), at risk for developing RB, not having RB, at risk for developing aggressive RB, at risk for not developing aggressive RB, is responding to treatment for RB, is not responding to treatment for RB, is/is not likely to respond to a particular RB treatment, or having/not having another disease or condition. In a specific embodiment, the term “comparing” refers to assessing whether the methylation level of one or more biomarkers of the present invention in a sample from a patient is the same as, more or less than, different from other otherwise correspond (or not) to methylation levels of the same biomarkers in a control sample (e.g., predefined levels that correlate to uninfected individuals, standard retina levels, etc.). As used herein, the terms “indicates” or “correlates” (or “indicating” or “correlating,” or “indication” or “correlation,” depending on the context) in reference to a parameter, e.g., a modulated proportion, level, or cellular localization in a sample from a patient, may mean that the patient has RB. In specific embodiments, the parameter may comprise the methylation status or level of one or more biomarkers of the present invention. A particular set or pattern of methylation of one or more biomarkers may indicate that a patient has RB (i.e., correlates to a patient having RB) or is at risk of developing RB. In other embodiments, a particular set or pattern of methylation of one or more biomarkers may be correlated to a patient being unaffected. In certain embodiments, “indicating,” or “correlating,” as used according to the present invention, may be by any linear or non-linear method of quantifying the relationship between methylation levels of biomarkers to a standard, control or comparative value for the assessment of the diagnosis, prediction of RB progression, assessment of efficacy of clinical treatment, identification of a patient that may respond to a particular treatment regime or pharmaceutical agent, monitoring of the progress of treatment, and in the context of a screening assay, for the identification of an anti-RB therapeutic.

The terms “patient,” “individual,” or “subject” are used interchangeably herein, and refer to a mammal, particularly, a human. In one aspect the patient is a human child, such as child of age of about two years or younger. The patient may have mild, intermediate or severe disease. The patient may be an individual, at risk of developing a disease, in need of treatment or in need of diagnosis based on particular symptoms or family history. In some cases, the terms may refer to treatment in experimental animals, in veterinary application, and in the development of animal models for disease, including, but not limited to, rodents including mice, rats, and hamsters, and primates. Mammals include, but are not limited to, humans, farm animals, sport animals and pets.

The term “retinoblastoma” (RB) refers to a disease in which malignant (cancer) cells form in the tissues of the retina. The retina is made of nerve tissue that lines the inside wall of the back of the eye. It receives light and converts the light into signals that travel down the optic nerve to the brain. The brain decodes the signals so that one can see the image. Retinoblastoma may be in one eye (unilateral) or in both eyes (bilateral). Cavitary retinoblastoma is a rare type of retinoblastoma in which cavities (hollow spaces) form within the tumor. Although retinoblastoma may occur at any age, it occurs most often in children younger than 2 years.

As used herein, “health care provider” includes either an individual or an institution that provides preventive, curative, promotional or rehabilitative health care services to a subject, such as a patient. In one embodiment, the data is provided to a health care provider so that they may use it in their diagnosis/treatment of the patient.

The term “gene” refers to a nucleic acid sequence that comprises control and coding sequences necessary for producing a polypeptide or precursor. The polypeptide may be encoded by a full-length coding sequence or by any portion of the coding sequence. A gene may contain one or more modifications in either the coding or the untranslated regions that could affect the biological activity or the chemical structure of the expression product, the rate of expression, or the manner of expression control. Such modifications include, but are not limited to, methylation, mutations, insertions, deletions, and substitutions of one or more nucleotides. The gene may constitute an uninterrupted coding sequence, or it may include one or more introns, bound by the appropriate splice junctions.

The term “gene expression” refers to the process by which a nucleic acid sequence undergoes successful transcription and/or translation such that detectable levels of the nucleotide sequence are expressed.

The terms “gene expression profile” or “gene signature” refer to a group of genes expressed by a particular cell or tissue type wherein presence of the genes taken together or the differential expression of such genes, is indicative/predictive of a certain condition; or one or more genes and their methylation status as compared to a control.

The term “nucleic acid” as used herein, refers to a molecule comprised of one or more nucleotides, i.e., ribonucleotides, deoxyribonucleotides, or both. The term includes monomers and polymers of ribonucleotides and deoxyribonucleotides, with the ribonucleotides and/or deoxyribonucleotides being bound together, in the case of the polymers, via 5′ to 3′ linkages. The ribonucleotide and deoxyribonucleotide polymers may be single or double-stranded. However, linkages may include any of the linkages known in the art including, for example, nucleic acids comprising 5′ to 3′ linkages. Furthermore, the term “nucleic acid sequences” contemplates the complementary sequence and specifically includes any nucleic acid sequence that is substantially homologous to the both the nucleic acid sequence and its complement.

The terms “array” and “microarray” refer to the type of genes represented on an array by oligonucleotides, and where the type of genes represented on the array is dependent on the intended purpose of the array (e.g., to monitor expression and/or methylation status of human genes). The oligonucleotides on a given array may correspond to the same type, category, or group of genes. Genes may be considered to be of the same type if they share some common characteristics such as species of origin (e.g., human, mouse, rat); disease state (e.g., cancer); functions (e.g., protein kinases, tumor suppressors); or same biological process (e.g., apoptosis, signal transduction, cell cycle regulation, proliferation, differentiation). For example, one array type may be a “cancer array” in which each of the array oligonucleotides correspond to a gene associated with a cancer.

The term “activation” as used herein refers to any alteration of a signaling pathway or biological response including, for example, increases above basal levels, restoration to basal levels from an inhibited state, and stimulation of the pathway above basal levels.

The term “differential expression” refers to both quantitative as well as qualitative differences in the temporal and tissue expression patterns of a gene in diseased tissues or cells versus normal adjacent tissue. For example, a differentially expressed gene may have its expression activated or partially or completely inactivated in normal versus disease conditions or may be up-regulated (over-expressed) or down-regulated (under-expressed) in a disease condition versus a normal condition. Such a qualitatively regulated gene may exhibit an expression pattern within a given tissue or cell type that is detectable in either control or disease conditions but is not detectable in both. Stated another way, a gene is differentially expressed when expression of the gene occurs at a higher or lower level in the diseased tissues or cells of a patient relative to the level of its expression in the normal (disease-free) tissues or cells of the patient and/or control tissues or cells. Also, as noted herein there is differential methylation.

The terms “measuring” and “determining” are used interchangeably throughout and refer to methods which include obtaining a patient sample and/or detecting the methylation status or level of a biomarker(s) in a sample. In one embodiment, the terms refer to obtaining a patient sample and detecting the methylation status or level of one or more biomarkers in the sample. In another embodiment, the terms “measuring” and “determining” mean detecting the methylation status or level of one or more biomarkers in a patient sample. Measuring can be accomplished by methods known in the art and those further described herein including, but not limited to, quantitative polymerase chain reaction (PCR). The term “measuring” is also used interchangeably throughout with the term “detecting.”

The term “methylation” refers to cytosine methylation at positions C5 or N4 of cytosine, the N6 position of adenine or other types of nucleic acid methylation. In vitro amplified DNA is unmethylated because in vitro DNA amplification methods do not retain the methylation pattern of the amplification template. However, “unmethylated DNA” or “methylated DNA” can also refer to amplified DNA whose original template was unmethylated or methylated, respectively. By “hypermethylation” or “elevated level of methylation” is meant an increase in methylation of a region of DNA (e.g., a biomarker of the present invention) that is considered statistically significant over levels of a control population. “Hypermethylation” or “elevated level of methylation” may refer to increased levels seen in a patient over time. By “hypomethylation” or “lowered level of methylation” is meant a decrease in methylation of a region of DNA (e.g., a biomarker of the present invention) that is considered statistically significant over levels of a control population. “Hypomethylation” or “lowered level of methylation” may refer to decreased levels seen in a patient over time.

In particular embodiments, a biomarker would be hypermethylated, as compared to a control, in a sample from a patient having or at risk of RB, such as at a methylation frequency of at least about 50%, at least about 60%, at least about 70%, at least about 75%, at least about 80%, at least about 85%, at least about 90%, at least about 95%, or about 100%. In other embodiments, a biomarker would be hypomethylated, as compared to a control, in a sample from a patient having or at risk of RB, such as at a methylation frequency of at least about 50%, at least about 60%, at least about 70%, at least about 75%, at least about 80%, at least about 85%, at least about 90%, at least about 95%, or about 100%.

A “methylation profile” refers to a set of data representing the methylation states or levels of one or more loci within a molecule of DNA from e.g., the genome of an individual or cells or sample from an individual. The profile can indicate the methylation state of every base in an individual, can comprise information regarding a subset of the base pairs (e.g., the methylation state of specific restriction enzyme recognition sequence) in a genome, or can comprise information regarding regional methylation density of each locus. In some embodiments, a methylation profile refers to the methylation states or levels of one or more biomarkers described herein. In more specific embodiments, a methylation profile refers to the methylation states or levels of the promoter regions or gene body regions described herein. In even more specific embodiments, a methylation profile refers to the methylation states of levels of CpG dinucleotides located within specific genes.

The terms “methylation status” or “methylation level” refers to the presence, absence and/or quantity of methylation at a particular nucleotide, or nucleotides within a portion of DNA. The methylation status of a particular DNA sequence (e.g., a DNA biomarker or DNA region as described herein) can indicate the methylation state of every base in the sequence or can indicate the methylation state of a subset of the base pairs (e.g., of cytosines or the methylation state of one or more specific restriction enzyme recognition sequences) within the sequence, or can indicate information regarding regional methylation density within the sequence without providing precise information of where in the sequence the methylation occurs. The methylation status can optionally be represented or indicated by a “methylation value” or “methylation level.” A methylation value or level can be generated, for example, by quantifying the amount of intact DNA present following restriction digestion with a methylation dependent restriction enzyme. In this example, if a particular sequence in the DNA is quantified using quantitative PCR, an amount of template DNA approximately equal to a mock treated control indicates the sequence is not highly methylated whereas an amount of template substantially less than occurs in the mock treated sample indicates the presence of methylated DNA at the sequence. Accordingly, a value, i.e., a methylation value, for example from the above-described example, represents the methylation status and can thus be used as a quantitative indicator of methylation status. This is of particular use when it is desirable to compare the methylation status of a sequence in a sample to a threshold value.

A “methylation-dependent restriction enzyme” refers to a restriction enzyme that cleaves or digests DNA at or in proximity to a methylated recognition sequence but does not cleave DNA at or near the same sequence when the recognition sequence is not methylated. Methylation-dependent restriction enzymes include those that cut at a methylated recognition sequence (e.g., DpnI) and enzymes that cut at a sequence near but not at the recognition sequence (e.g., McrBC). For example, McrBC's recognition sequence is 5′ RmC (N40-3000) RmC 3′ where “R” is a purine and “mC” is a methylated cytosine and “N40-3000” indicates the distance between the two RmC half sites for which a restriction event has been observed. McrBC generally cuts close to one half-site or the other, but cleavage positions are typically distributed over several base pairs, approximately 30 base pairs from the methylated base. McrBC sometimes cuts 3′ of both half sites, sometimes 5′ of both half sites, and sometimes between the two sites. Exemplary methylation-dependent restriction enzymes include, e.g., McrBC, McrA, MrrA, BisI, GlaI and DpnI. One of skill in the art will appreciate that any methylation-dependent restriction enzyme, including homologs and orthologs of the restriction enzymes described herein, is also suitable for use in the present invention.

A “methylation-sensitive restriction enzyme” refers to a restriction enzyme that cleaves DNA at or in proximity to an unmethylated recognition sequence but does not cleave at or in proximity to the same sequence when the recognition sequence is methylated. Exemplary methylation-sensitive restriction enzymes are described in, e.g., McClelland et al., 22 (17) NUCLEIC ACIDS RES. 3640-59 (1994) and http://rebase.neb.com. Suitable methylation-sensitive restriction enzymes that do not cleave DNA at or near their recognition sequence when a cytosine within the recognition sequence is methylated at position C5 include, e.g., Aat II, Aci I, Acd I, Age I, Alu I, Asc I, Ase I, AsiS I, Bbe I, BsaA I, BsaH I, BsiE I, BsiW I, BsrF I, BssH II, BssK I, BstB I, BstN I, BstU I, Cla I, Eae I, Eag I, Fau I, Fse I, Hha I, HinP1 I, HinC II, Hpa II, Hpy99 I, HpyCH4 IV, Kas I, Mbo I, Mlu I, MapA1 I, Msp I, Nae I, Nar I, Not I, Pm1 I, Pst I, Pvu I, Rsr II, Sac II, Sap I, Sau3A I, Sfl I, Sfo I, SgrA I, Sma I, SnaB I, Tsc I, Xma I, and Zra I. Suitable methylation-sensitive restriction enzymes that do not cleave DNA at or near their recognition sequence when an adenosine within the recognition sequence is methylated at position N6 include, e.g., Mbo I. One of skill in the art will appreciate that any methylation-sensitive restriction enzyme, including homologs and orthologs of the restriction enzymes described herein, is also suitable for use in the present invention. One of skill in the art will further appreciate that a methylation-sensitive restriction enzyme that fails to cut in the presence of methylation of a cytosine at or near its recognition sequence may be insensitive to the presence of methylation of an adenosine at or near its recognition sequence. Likewise, a methylation-sensitive restriction enzyme that fails to cut in the presence of methylation of an adenosine at or near its recognition sequence may be insensitive to the presence of methylation of a cytosine at or near its recognition sequence. For example, Sau3AI is sensitive (i.e., fails to cut) to the presence of a methylated cytosine at or near its recognition sequence, but is insensitive (i.e., cuts) to the presence of a methylated adenosine at or near its recognition sequence. One of skill in the art will also appreciate that some methylation-sensitive restriction enzymes are blocked by methylation of bases on one or both strands of DNA encompassing of their recognition sequence, while other methylation-sensitive restriction enzymes are blocked only by methylation on both strands but can cut if a recognition site is hemi-methylated.

The terms “sample,” “patient sample,” “biological sample,” and the like, encompass a variety of sample types obtained from a patient, individual, or subject and can be used in a diagnostic or monitoring assay. The patient sample may be obtained from a healthy subject, a diseased patient or a patient having associated symptoms of RB. Moreover, a sample obtained from a patient can be divided and only a portion may be used for diagnosis. Further, the sample, or a portion thereof, can be stored under conditions to maintain sample for later analysis. The definition specifically encompasses blood, aqueous humor (AH; the clear liquid inside the front part of the eye) and other liquid samples of biological origin (including, but not limited to, peripheral blood, serum, plasma, urine, saliva and synovial fluid), solid tissue samples such as a biopsy specimen (e.g., from an enucleated tumor) or tissue cultures or cells derived therefrom and the progeny thereof. In a specific embodiment, a sample comprises an AH sample. Samples may be collected as part of routine physician visits, e.g., at the doctor's office. The definition also includes samples that have been manipulated in any way after their procurement, such as by centrifugation, filtration, precipitation, dialysis, chromatography, treatment with reagents, washed, or enriched for certain cell or DNA populations. The terms further encompass a clinical sample, and also include cells in culture, cell supernatants, tissue samples, organs, and the like. Samples may also comprise fresh-frozen and/or formalin-fixed, paraffin-embedded tissue blocks, such as blocks prepared from clinical or pathological biopsies, prepared for pathological analysis or study by immunohistochemistry.

Treatments for RB include, but are not limited to, cryotherapy, thermotherapy, chemotherapy (systemic and regional (e.g., ophthalmic artery infusion, intravitreal chemotherapy, or intrathecal chemotherapy), radiation therapy (e.g., external beam radiation therapy, intensity-modulated radiation therapy (IMRT), internal radiation therapy (including plaque radiotherapy)), high-dose chemotherapy with stem cell rescue, surgery (enucleation), targeted therapy (including oncolytic virus therapy). See also FIG. 10.

Various methodologies of the instant invention include a step that involves comparing a value, level, feature, characteristic, property, etc. to a “suitable control,” referred to interchangeably herein as an “appropriate control” or a “control sample.” A “suitable control,” “appropriate control” or a “control sample” is any control or standard familiar to one of ordinary skill in the art useful for comparison purposes. In one embodiment, a “suitable control” or “appropriate control” is a value, level, feature, characteristic, property, etc., determined in a liquid sample, cell, organ, or patient, e.g., a control or normal cell, organ, or patient, exhibiting, for example, normal traits. For example, the biomarkers of the present invention may be assayed for their methylation level in a sample from an unaffected individual (UI) or a normal control individual (NC) (both terms are used interchangeably herein). In another embodiment, a “suitable control” or “appropriate control” is a value, level, feature, characteristic, property, etc. determined prior to performing a therapy (e.g., a RB treatment) on a patient. In a further embodiment, a “suitable control” or “appropriate control” is a predefined value, level, feature, characteristic, property, etc. A “suitable control” can be a methylation profile of one or more biomarkers of the present invention that correlates to RB, to which a patient sample can be compared. The patient sample can also be compared to a negative control, i.e., a methylation profile that correlates to not having RB.

### II. Hypermethylated/Hypomethylated Biomarkers and Detection Thereof

The biomarkers of the present invention are differentially methylated in RB versus normal retina. Such biomarkers can be used individually as diagnostic tool, or in combination as a biomarker panel.

The DNA biomarkers of the present invention can comprise fragments of a polynucleotide (e.g., regions of genome polynucleotide or DNA) which likely contain CpG island(s), or fragments which are more susceptible to methylation or demethylation than other regions of genome DNA. The term “CpG islands” is a region of genome DNA which shows higher frequency of 5′-CG-3′ (CpG) dinucleotides than other regions of genome DNA. Methylation of DNA at CpG dinucleotides, in particular, the addition of a methyl group to position 5 of the cytosine ring at CpG dinucleotides, is one of the epigenetic modifications in mammalian cells. CpG islands often harbor the promoters of genes and play a pivotal role in the control of gene expression. A subset of islands can become methylated or unmethylated during the development of a disease or condition (e.g., RB).

There are a number of methods that can be employed to measure, detect, determine, identify, and characterize the methylation status/level of a biomarker (i.e., a region/fragment of DNA or a region/fragment of genome DNA (e.g., CpG island-containing region/fragment)) in the development of a disease or condition (e.g., RB) and thus diagnose the onset, presence or status of the disease or condition.

In some embodiments, methods for detecting methylation include randomly shearing or randomly fragmenting the genomic DNA, cutting the DNA with a methylation-dependent or methylation-sensitive restriction enzyme and subsequently selectively identifying and/or analyzing the cut or uncut DNA. Selective identification can include, for example, separating cut and uncut DNA (e.g., by size) and quantifying a sequence of interest that was cut or, alternatively, that was not cut. See, e.g., U.S. Pat. No. 7,186,512. Alternatively, the method can encompass amplifying intact DNA after restriction enzyme digestion, thereby only amplifying DNA that was not cleaved by the restriction enzyme in the area amplified. See, e.g., U.S. Pat. Nos. 7,910,296; 7,901,880; and 7,459,274. In some embodiments, amplification can be performed using primers that are gene specific. Alternatively, adaptors can be added to the ends of the randomly fragmented DNA, the DNA can be digested with a methylation-dependent or methylation-sensitive restriction enzyme, intact DNA can be amplified using primers that hybridize to the adaptor sequences. In this case, a second step can be performed to determine the presence, absence or quantity of a particular gene in an amplified pool of DNA. In some embodiments, the DNA is amplified using real-time, quantitative PCR.

In other embodiments, the methods comprise quantifying the average methylation density in a target sequence within a population of genomic DNA. In some embodiments, the method comprises contacting genomic DNA with a methylation-dependent restriction enzyme or methylation-sensitive restriction enzyme under conditions that allow for at least some copies of potential restriction enzyme cleavage sites in the locus to remain uncleaved; quantifying intact copies of the locus; and comparing the quantity of amplified product to a control value representing the quantity of methylation of control DNA, thereby quantifying the average methylation density in the locus compared to the methylation density of the control DNA.

The quantity of methylation of a locus of DNA can be determined by providing a sample of genomic DNA comprising the locus, cleaving the DNA with a restriction enzyme that is either methylation-sensitive or methylation-dependent, and then quantifying the amount of intact DNA or quantifying the amount of cut DNA at the DNA locus of interest. The amount of intact or cut DNA will depend on the initial amount of genomic DNA containing the locus, the amount of methylation in the locus, and the number (i.e., the fraction) of nucleotides in the locus that are methylated in the genomic DNA. The amount of methylation in a DNA locus can be determined by comparing the quantity of intact DNA or cut DNA to a control value representing the quantity of intact DNA or cut DNA in a similarly treated DNA sample. The control value can represent a known or predicted number of methylated nucleotides. Alternatively, the control value can represent the quantity of intact or cut DNA from the same locus in another (e.g., normal, non-diseased) cell or a second locus.

By using at least one methylation-sensitive or methylation-dependent restriction enzyme under conditions that allow for at least some copies of potential restriction enzyme cleavage sites in the locus to remain uncleaved and subsequently quantifying the remaining intact copies and comparing the quantity to a control, average methylation density of a locus can be determined. If the methylation-sensitive restriction enzyme is contacted to copies of a DNA locus under conditions that allow for at least some copies of potential restriction enzyme cleavage sites in the locus to remain uncleaved, then the remaining intact DNA will be directly proportional to the methylation density, and thus may be compared to a control to determine the relative methylation density of the locus in the sample. Similarly, if a methylation-dependent restriction enzyme is contacted to copies of a DNA locus under conditions that allow for at least some copies of potential restriction enzyme cleavage sites in the locus to remain uncleaved, then the remaining intact DNA will be inversely proportional to the methylation density, and thus may be compared to a control to determine the relative methylation density of the locus in the sample. Such assays are disclosed in, e.g., U.S. Pat. No. 7,910,296.

Quantitative amplification methods (e.g., quantitative PCR or quantitative linear amplification) can be used to quantify the amount of intact DNA within a locus flanked by amplification primers following restriction digestion. Methods of quantitative amplification are disclosed in, e.g., U.S. Pat. Nos. 6,180,349; 6,033,854; and 5,972,602, as well as in, e.g., DeGraves, et al., 34 (1) Biotechniques 106-15 (2003); Deiman B, et al., 20 (2) MOL. BIOTECHNOL. 163-79 (2002); and Gibson et al., 6 Genome Research 995-1001 (1996). Amplifications may be monitored in “real time.”

Additional methods for detecting DNA methylation can involve genomic sequencing before and after treatment of the DNA with bisulfite. See, e.g., Frommer et al., 89 Proc. Natl. Acad. Sci. USA 1827-31 (1992). When sodium bisulfite is contacted to DNA, unmethylated cytosine is converted to uracil, while methylated cytosine is not modified. In some embodiments, restriction enzyme digestion of PCR products amplified from bisulfite-converted DNA is used to detect DNA methylation. See, e.g., Xiong & Laird, 25 Nucleic Acids Res. 2532-34 (1997); and Sadri & Hornsby, 24 Nucl. Acids Res. 5058-59 (1996).

In some embodiments, a MethyLight assay is used alone or in combination with other methods to detect DNA methylation. See, Eads et al., 59 Cancer Res. 2302-06 (1999). Briefly, in the MethyLight process genomic DNA is converted in a sodium bisulfite reaction (the bisulfite process converts unmethylated cytosine residues to uracil). Amplification of a DNA sequence of interest is then performed using PCR primers that hybridize to CpG dinucleotides. By using primers that hybridize only to sequences resulting from bisulfite conversion of unmethylated DNA, (or alternatively to methylated sequences that are not converted) amplification can indicate methylation status of sequences where the primers hybridize. Similarly, the amplification product can be detected with a probe that specifically binds to a sequence resulting from bisulfite treatment of a unmethylated (or methylated) DNA. If desired, both primers and probes can be used to detect methylation status. Thus, kits for use with MethyLight can include sodium bisulfite as well as primers or detectably labeled probes (including but not limited to Taqman or molecular beacon probes) that distinguish between methylated and unmethylated DNA that have been treated with bisulfite. Other kit components can include, e.g., reagents necessary for amplification of DNA including but not limited to, PCR buffers, deoxynucleotides, and a thermostable polymerase.

In other embodiments, a Methylation-sensitive Single Nucleotide Primer Extension (Ms-SNuPE) reaction is used alone or in combination with other methods to detect DNA methylation. See Gonzalgo & Jones, 25 Nucleic Acids Res. 2529-31 (1997). The Ms-SNuPE technique is a quantitative method for assessing methylation differences at specific CpG sites based on bisulfite treatment of DNA, followed by single-nucleotide primer extension. Briefly, genomic DNA is reacted with sodium bisulfite to convert unmethylated cytosine to uracil while leaving 5-methylcytosine unchanged. Amplification of the desired target sequence is then performed using PCR primers specific for bisulfate-converted DNA, and the resulting product is isolated and used as a template for methylation analysis at the CpG site(s) of interest. Typical reagents (e.g., as might be found in a typical Ms-SNuPE-based kit) for Ms-SNuPE analysis can include, but are not limited to: PCR primers for specific gene (or methylation-altered DNA sequence or CpG island); optimized PCR buffers and deoxynucleotides; gel extraction kit; positive control primers; Ms-SNuPE primers for a specific gene; reaction buffer (for the Ms-SNuPE reaction); and detectably-labeled nucleotides. Additionally, bisulfite conversion reagents may include DNA denaturation buffer; sulfonation buffer; DNA recovery regents or kit (e.g., precipitation, ultrafiltration, affinity column); desulfonation buffer; and DNA recovery components.

In further embodiments, a methylation-specific PCR reaction is used alone or in combination with other methods to detect DNA methylation. A methylation-specific PCR assay entails initial modification of DNA by sodium bisulfite, converting all unmethylated, but not methylated, cytosines to uracil, and subsequent amplification with primers specific for methylated versus unmethylated DNA. See, Herman et al., 93 Proc. Natl. Acad. Sci. USA 9821-26, (1996); and U.S. Pat. No. 5,786,146.

Additional methylation detection methods include, but are not limited to, methylated CpG island amplification (see, Toyota et al., 59 Cancer Res. 2307-12 (1999)) and those methods described in, e.g., U.S. Pat. Nos. 7,553,627; 6,331,393; U.S. patent Ser. No. 12/476,981; U.S. Patent Publication No. 2005/0069879; Rein, et al., 26 (10) Nucleic Acids Res. 2255-64 (1998); and Olek et al., 17 (3) Nat. Genet. 275-6 (1997).

In some embodiments DNA methylation detection is performed with the Illumina Infinium Methylation Assay (or similar commercially available instruments & technology) using a custom designed chip which uses ‘BeadChip’ technology to generate a selective analysis of human DNA methylation patterns. Similar to bisulfite sequencing and pyrosequencing, this method quantifies methylation levels at various loci within the genome. The processing and analysis of the Illumina Infinium assay is summarized below:

### Bisulfite Treatment

Approximately 1 μg of genomic DNA is used in bisulfite conversion to convert the unmethylated cytosine into uracil. The product contains unconverted cytosine where they were previously methylated, but cytosine is converted to uracil if they were previously unmethylated.

### Whole-Genomic DNA Amplification

The bisulfite treated DNA is subjected to whole-genome multiple displacement amplification via random hexamer priming and @29 DNA polymerase, which has a proofreading activity resulting in error rates 100 times lower than the Taq polymerase. The products are then enzymatically fragmented, purified from dNTPs, primers and enzymes, and applied to the chip.

### Hybridization and Single-Base Extension

On the chip, there are two bead types for each CpG site per locus. Each locus tested is differentiated by different bead types. Both bead types are attached to single-stranded 50-mer DNA oligonucleotides that differ in sequence only at the free end; this type of probe is known as an allele-specific oligonucleotide. One of the bead types will correspond to the methylated cytosine locus and the other will correspond to the unmethylated cytosine locus, which has been converted into uracil during bisulfite treatment and later amplified as thymine during whole-genome amplification. The bisulfite-converted amplified DNA products are denatured into single strands and hybridized to the chip via allele-specific annealing to either the methylation-specific probe or the non-methylation probe. Hybridization is followed by single base extension with hapten-labeled dideoxynucleotides. The ddCTP and ddGTP are labeled with biotin while ddATP and ddUTP are labeled with 2,4-dinitrophenol (DNP).

### Fluorescence Staining and Scanning of Chip

After incorporation of these hapten-labeled ddNTPs, multilayered immunohistochemical assays are performed by repeated rounds of staining with a combination of antibodies to differentiate the two types. After staining, the chip is scanned to show the intensities of the unmethylated and methylated bead types. The raw data are analyzed by the proprietary software, and the fluorescence intensity ratios between the two bead types are calculated. For a given individual at a given locus, a ratio value of 0 equals to non-methylation of the locus (i.e., homozygous unmethylated); a ratio of 1 equals to total methylation (i.e., homozygous methylated); and a value of 0.5 means that one copy is methylated and the other is not (i.e., heterozygosity), in the diploid human genome.

### Analysis of Methylation Data

The scanned microarray images of methylation data are further analyzed by the system, which normalizes the raw data to reduce the effects of experimental variation, background and average normalization, and performs standard statistical tests on the results. The data can then be compiled into several types of figures for visualization and analysis.

### Custom Chip Validation Studies

Prior to using the custom chip in clinical studies, the chip is validated at a CLIA certified vendor by running 25 duplicate samples run on the custom methylation array which are paired with at least 25 samples from the EPIC 850k array to provide the necessary data for validation.

### III. Determination of RB Status

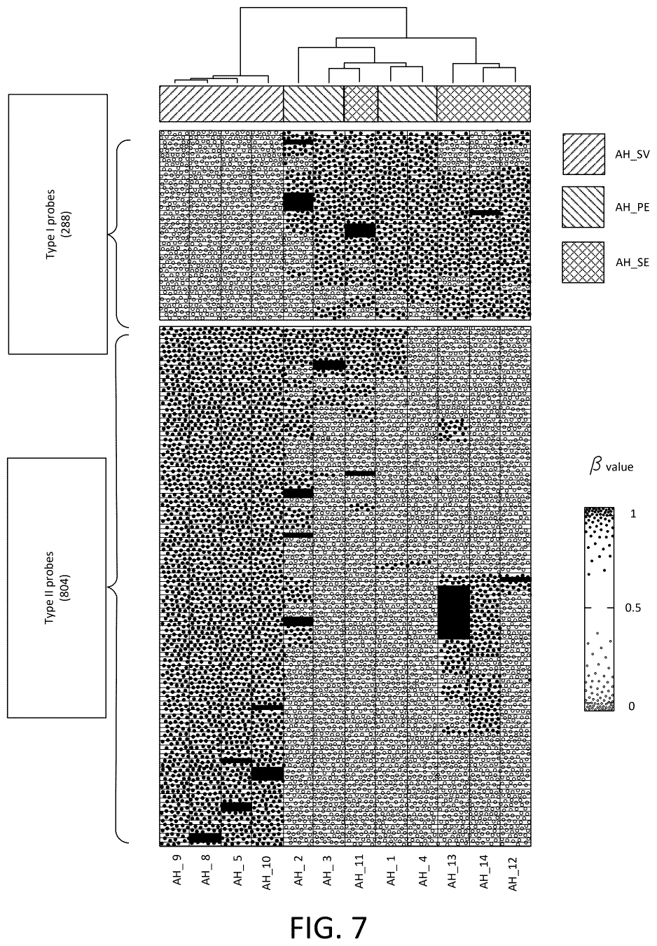

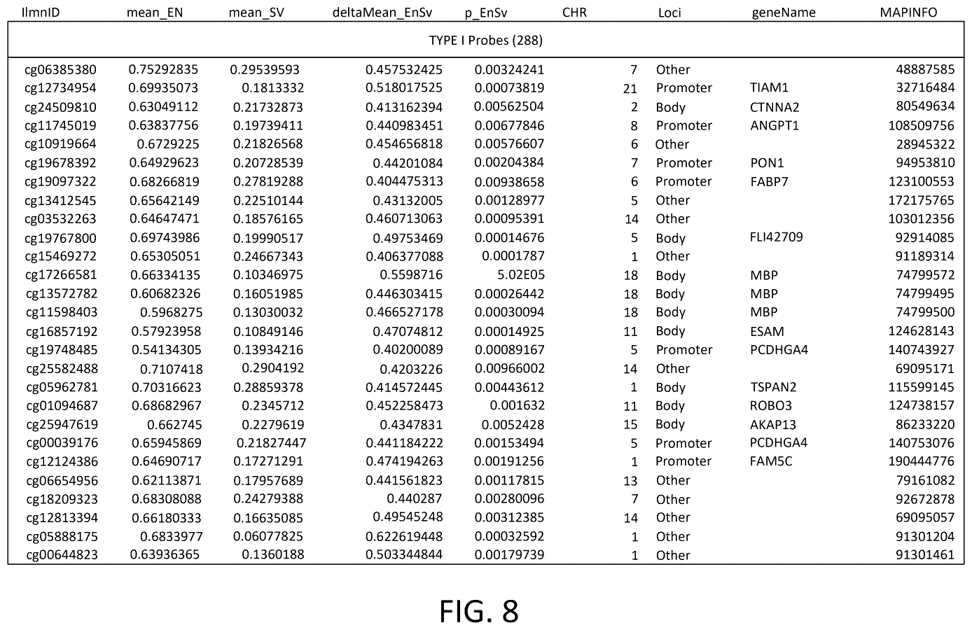

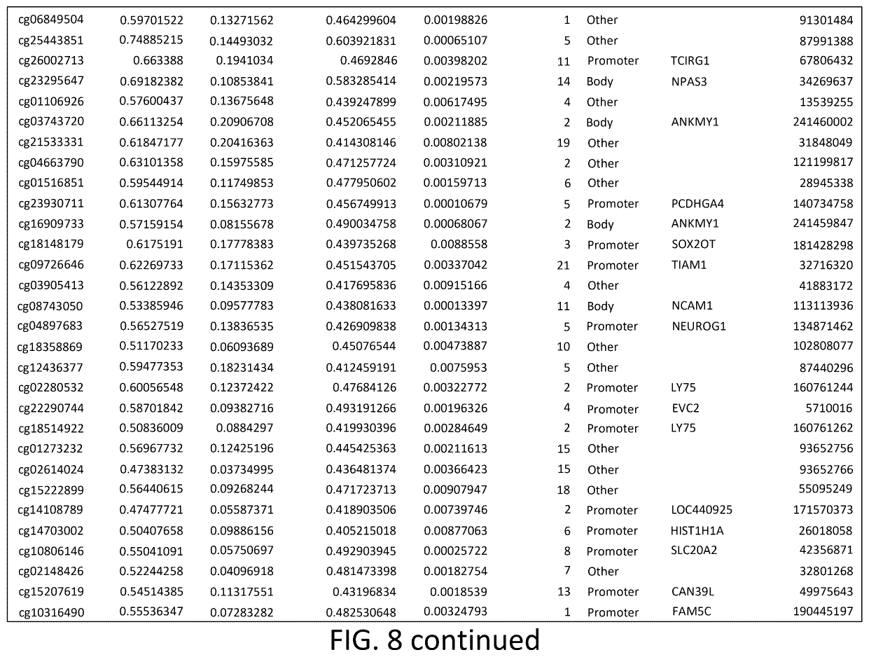

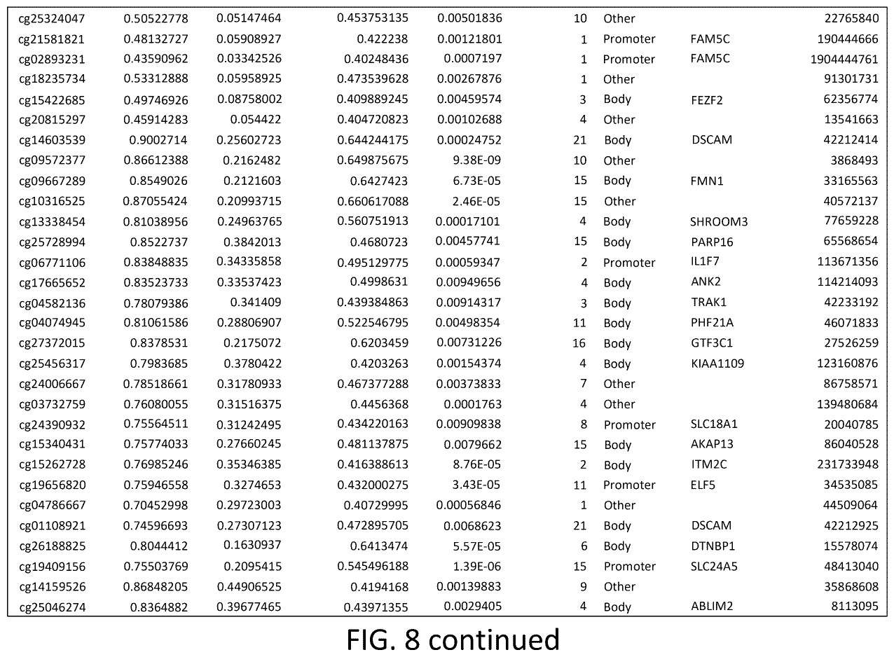

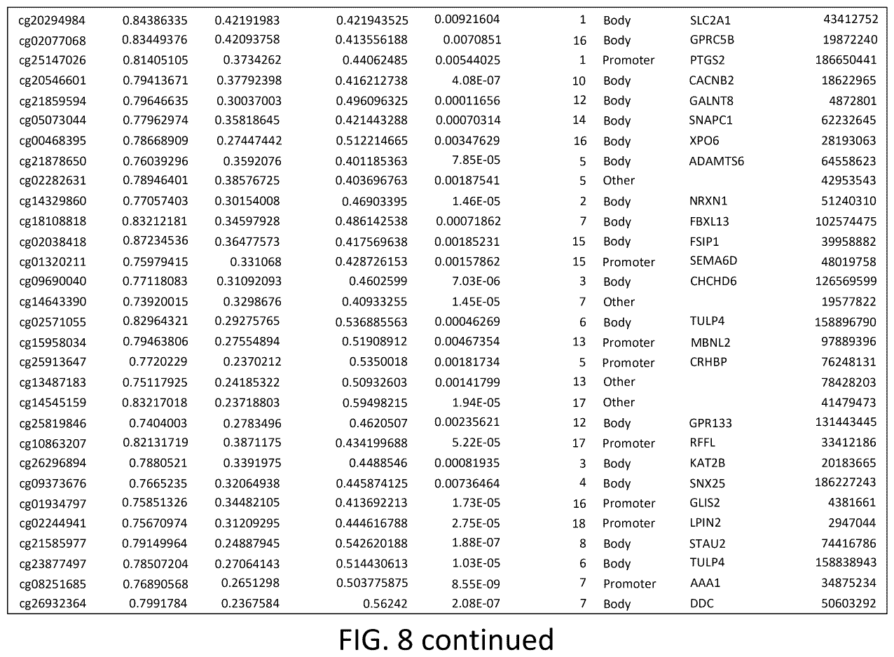

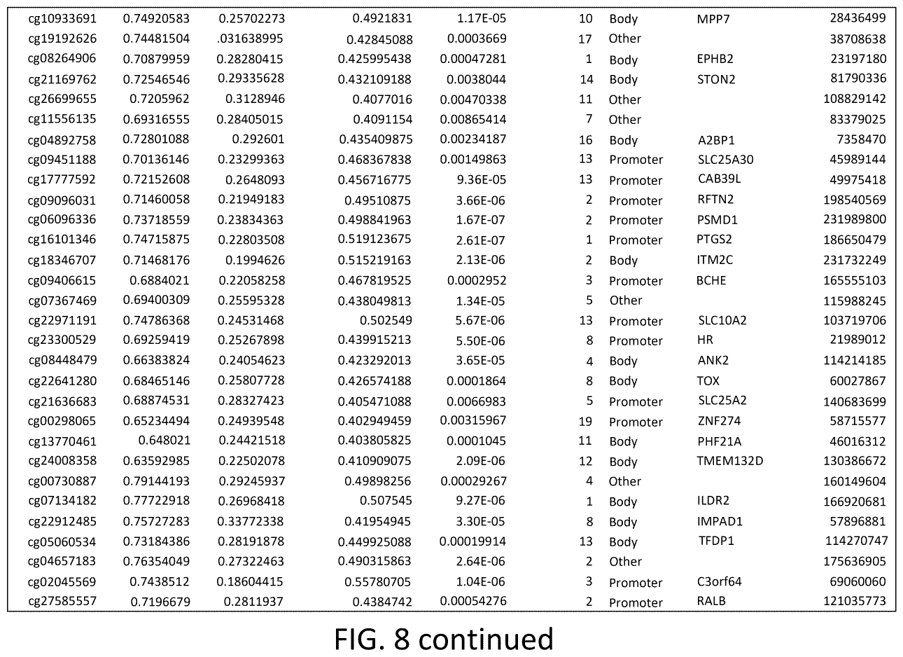

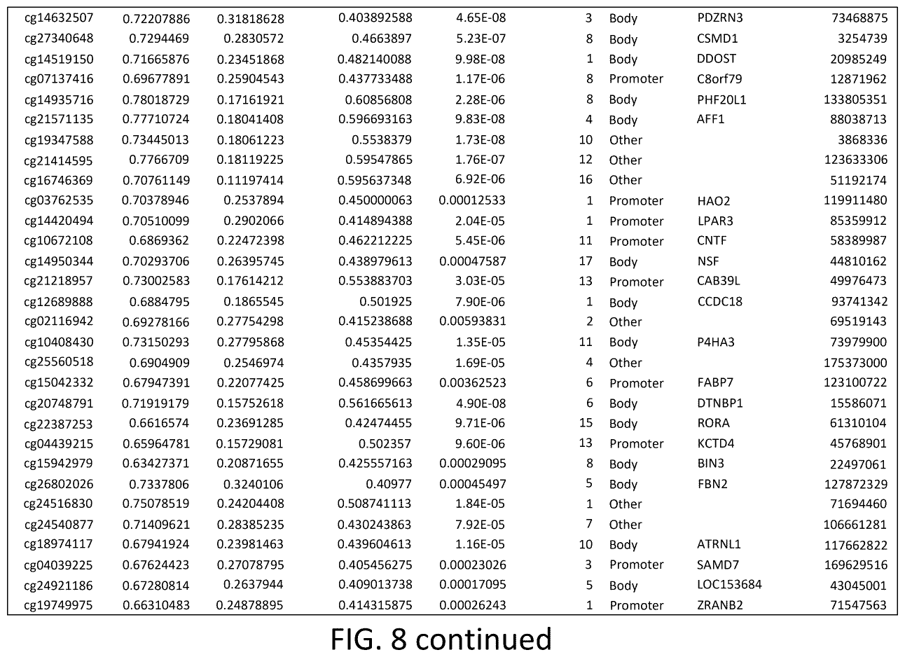

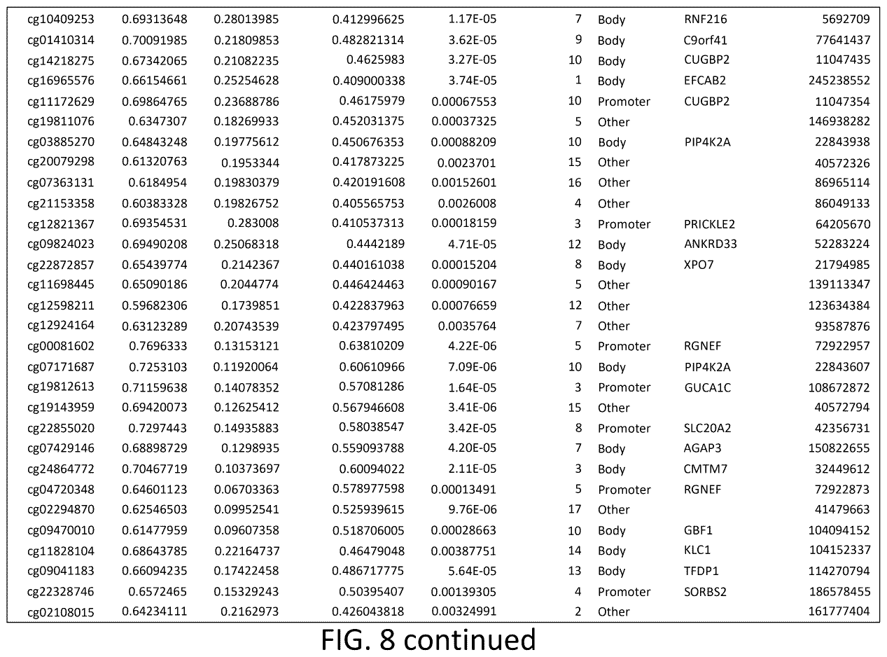

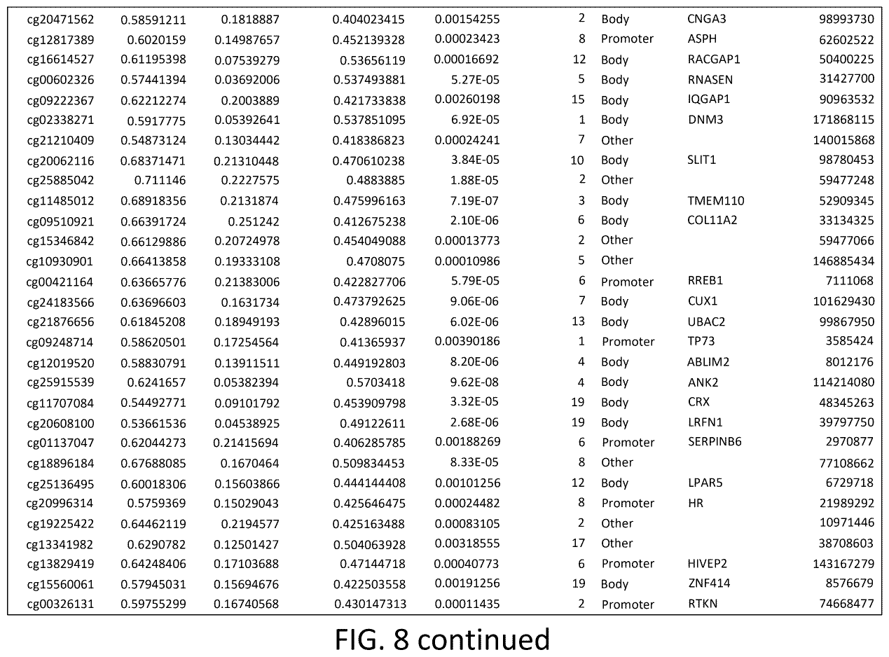

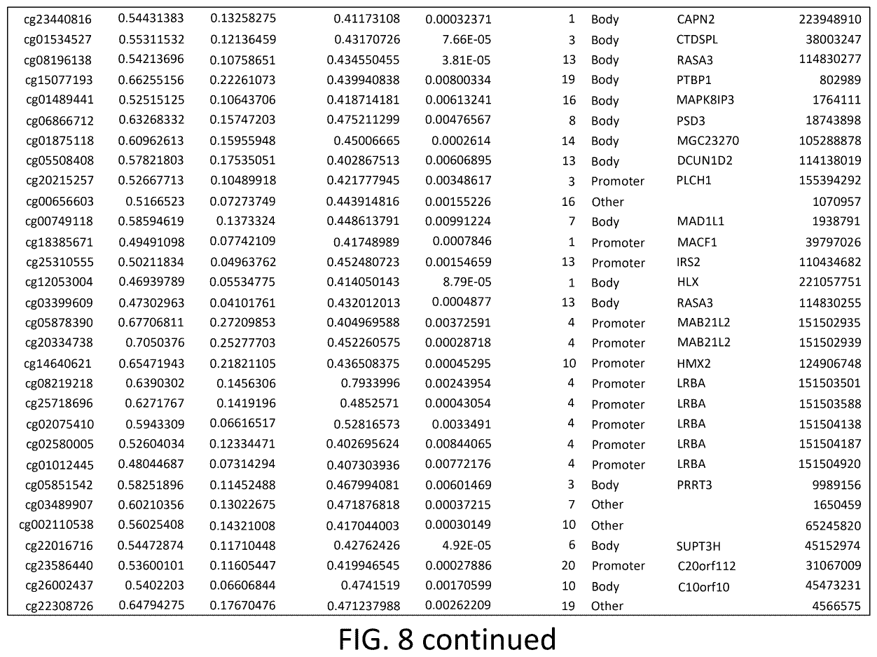

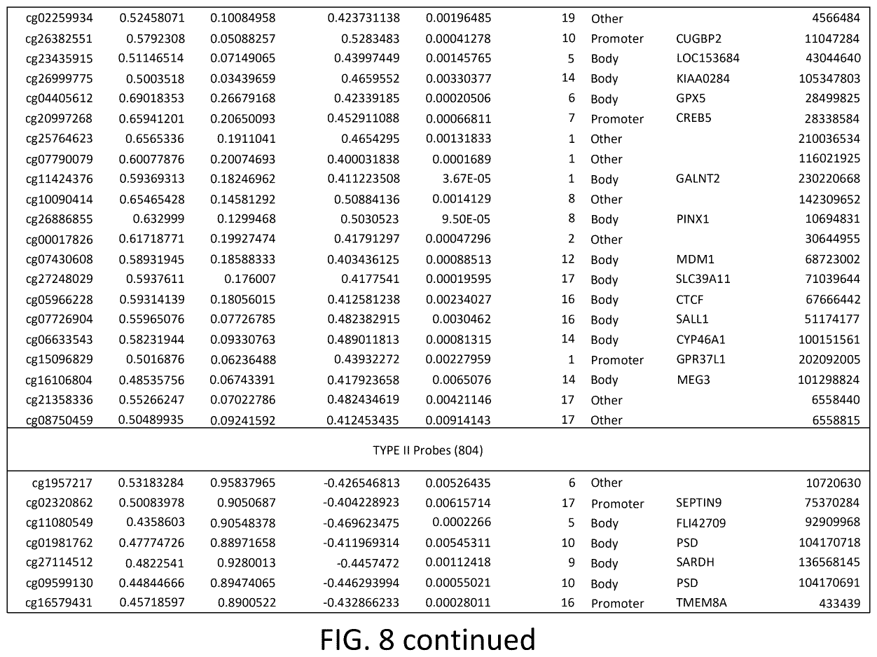

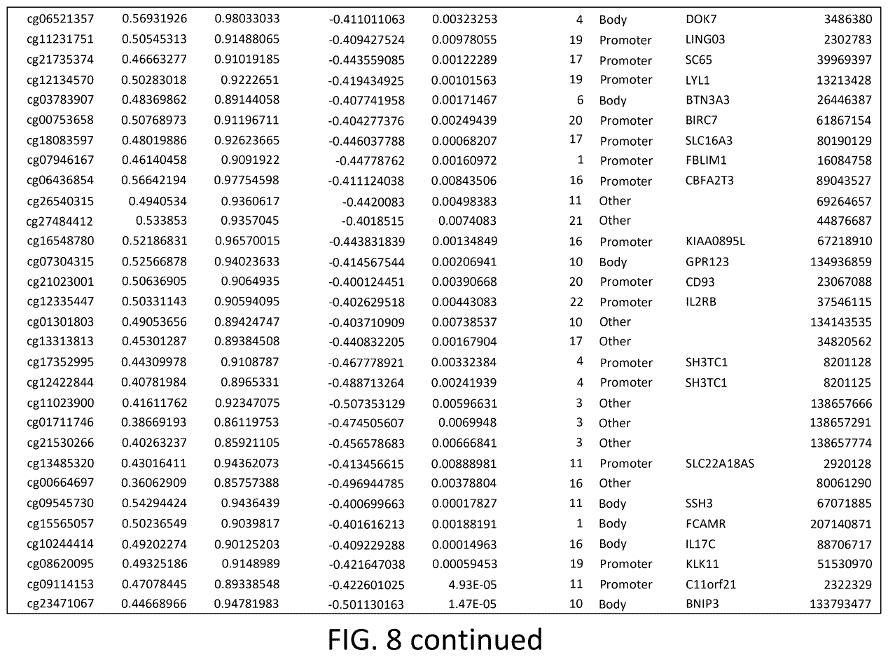

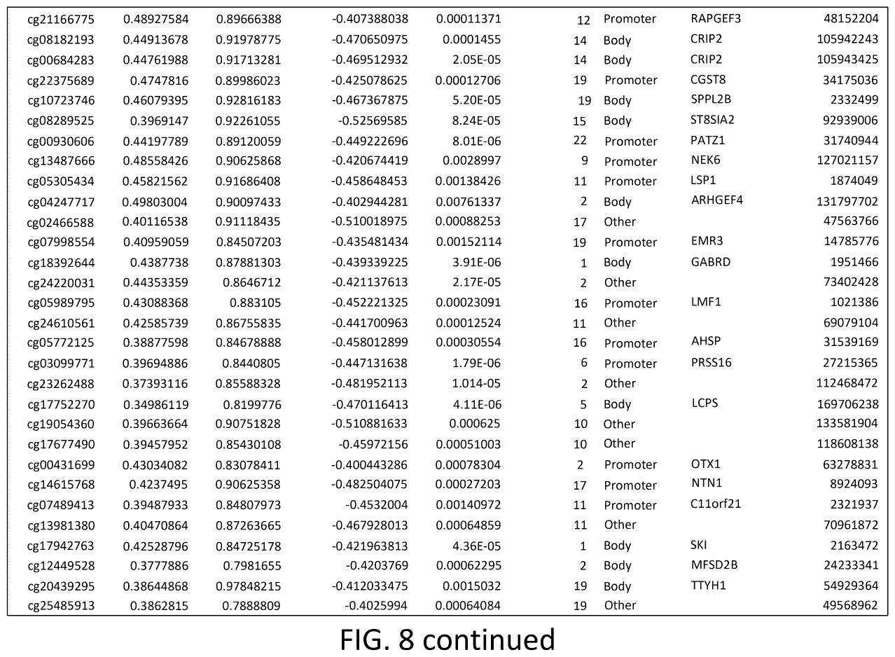

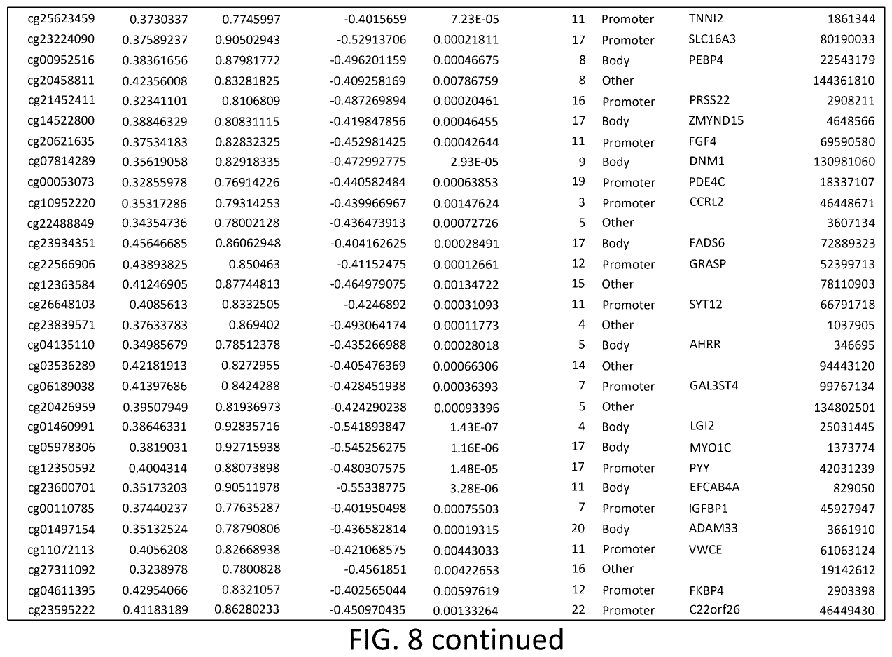

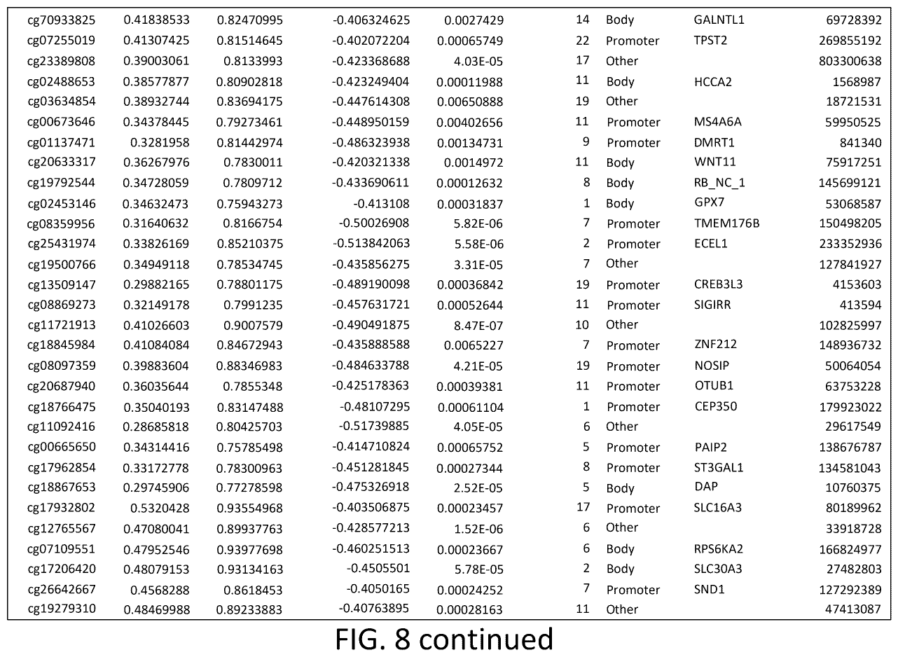

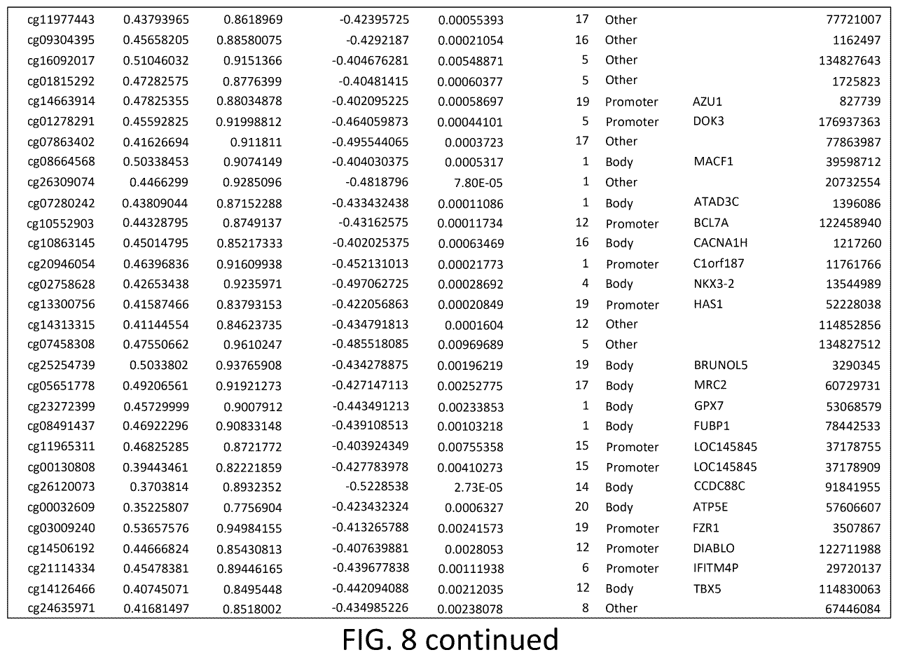

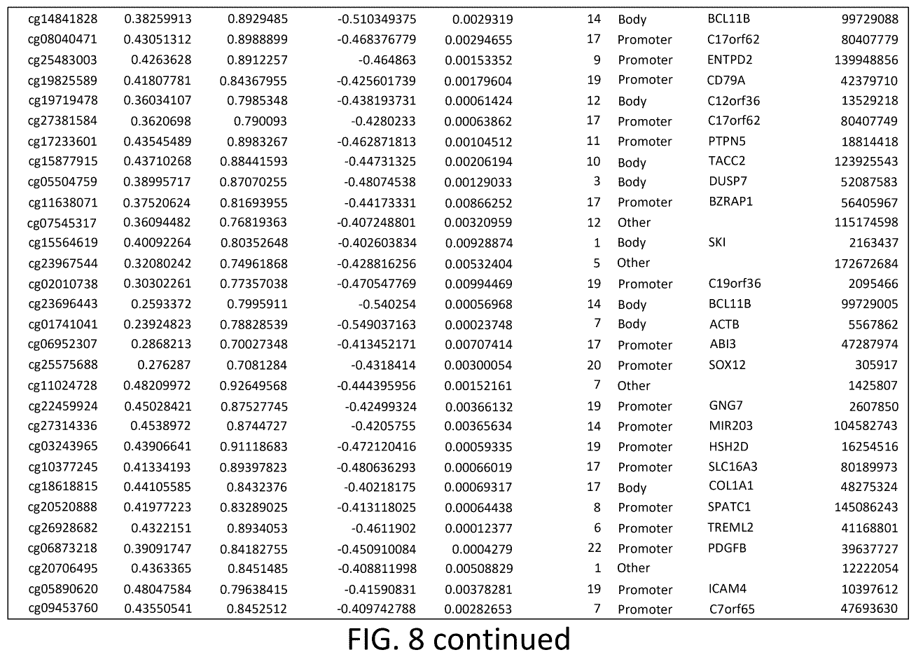

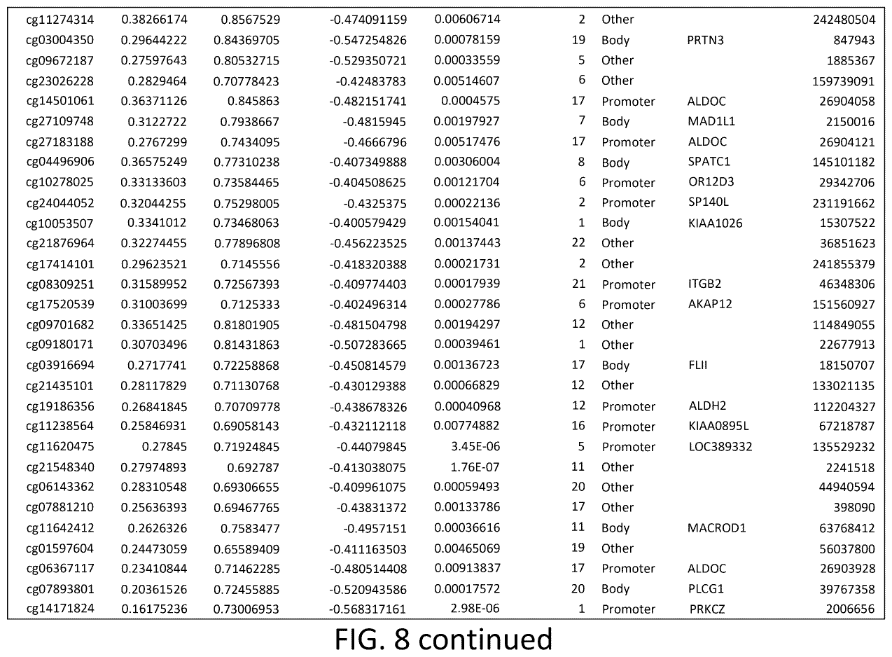

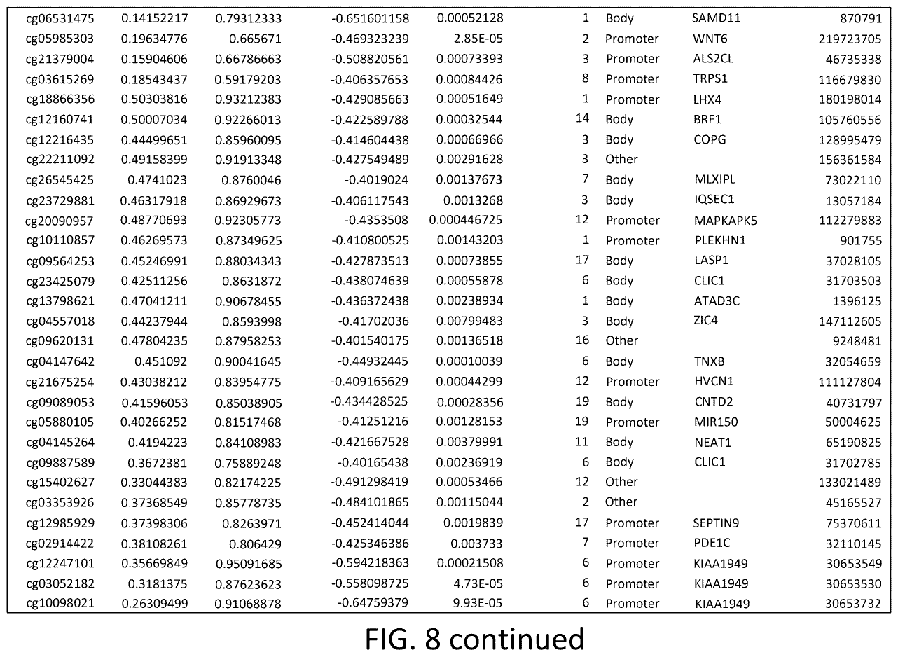

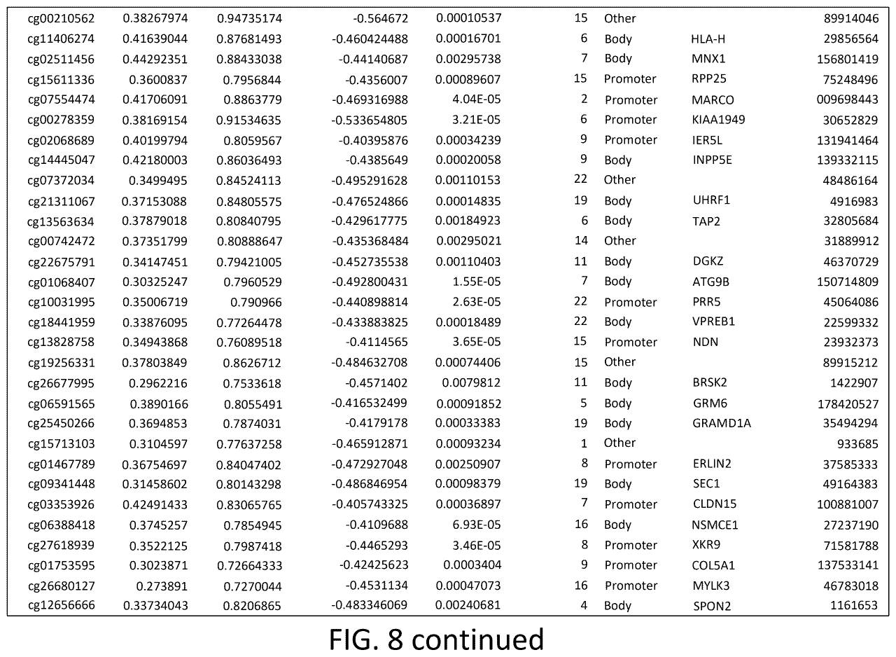

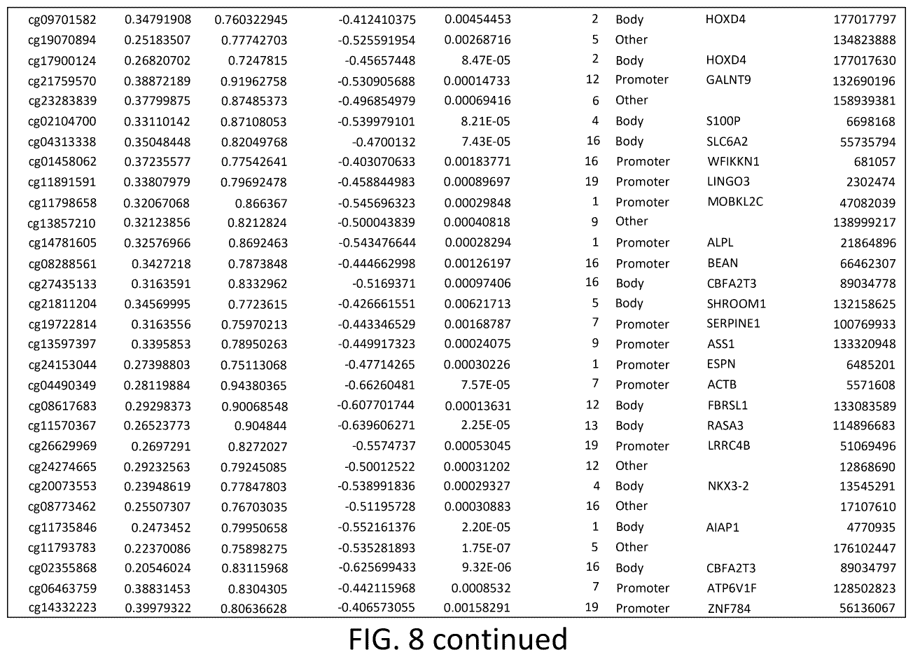

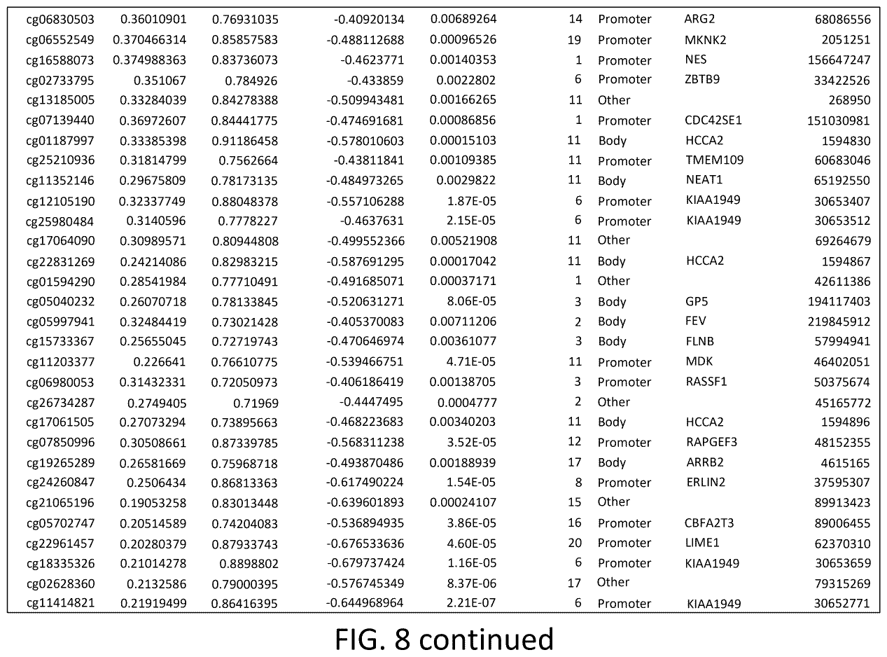

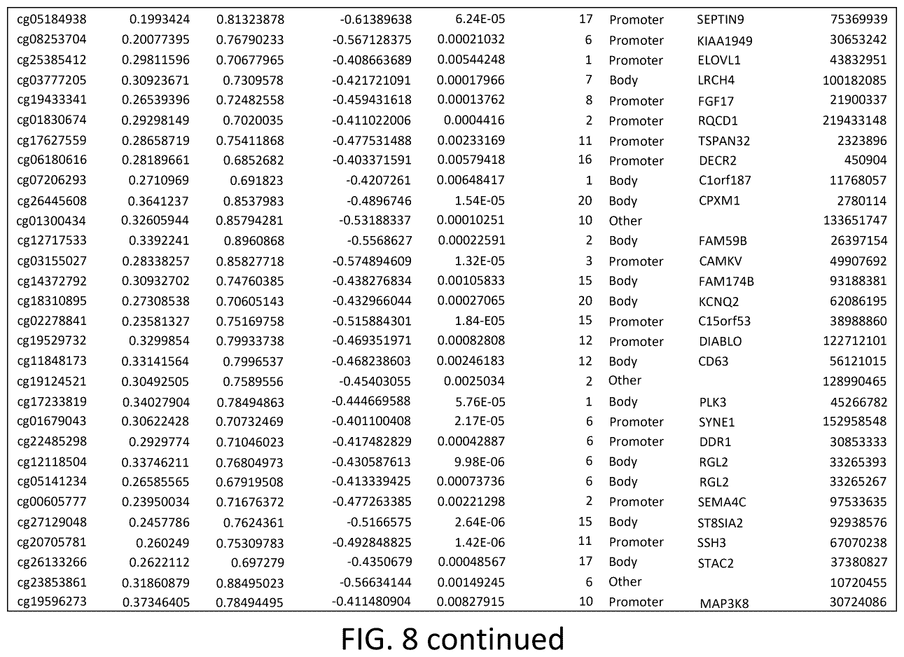

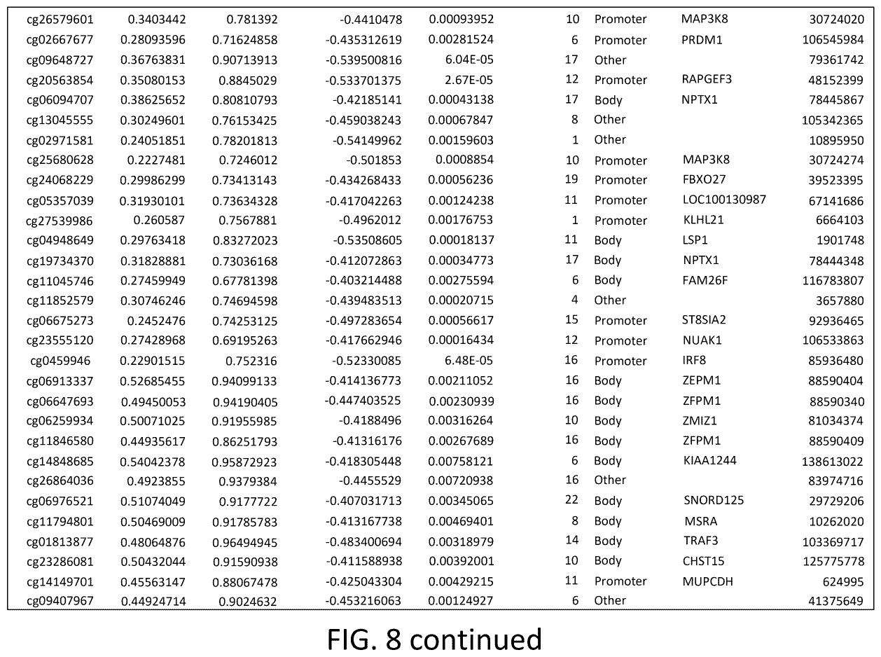

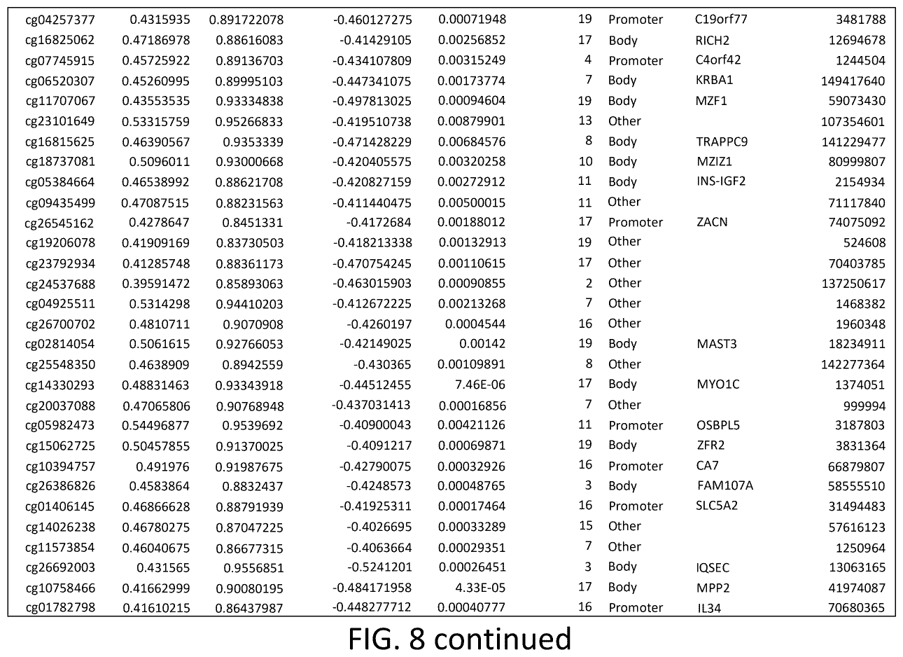

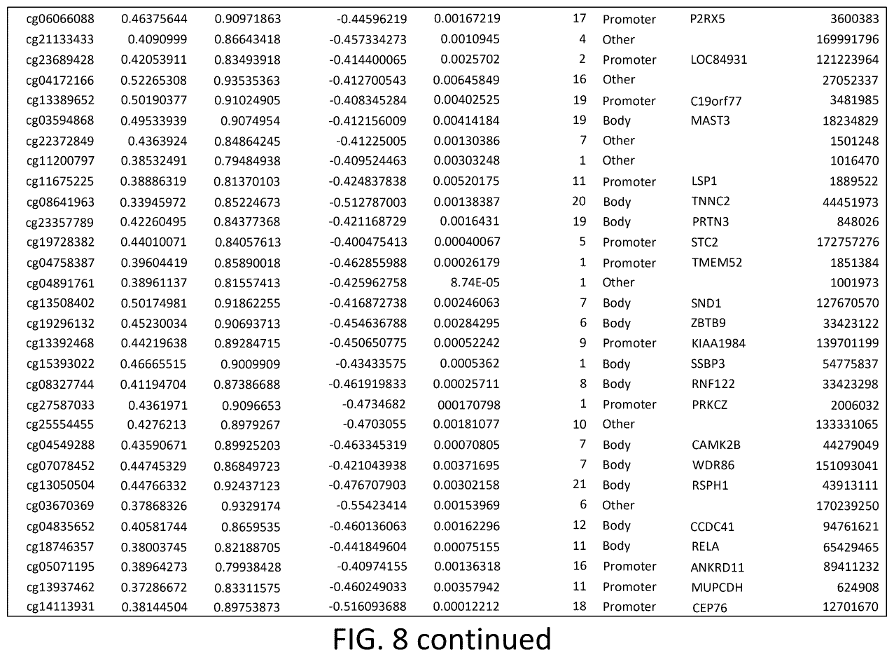

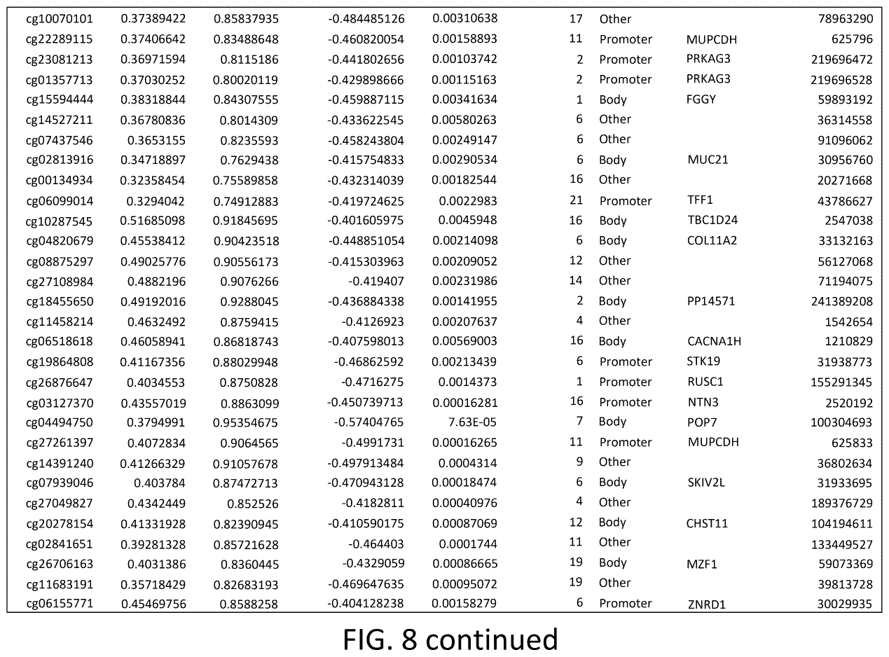

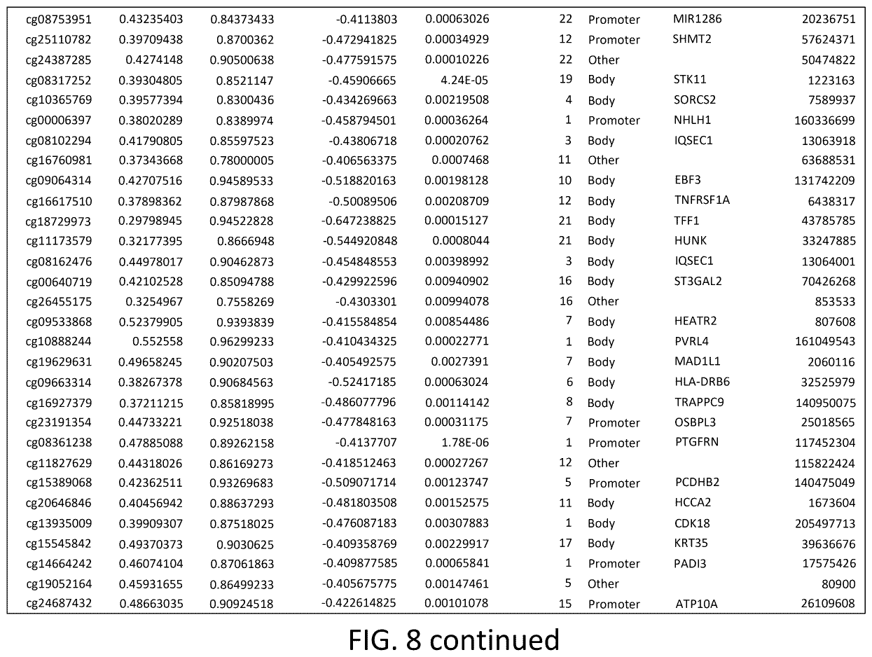

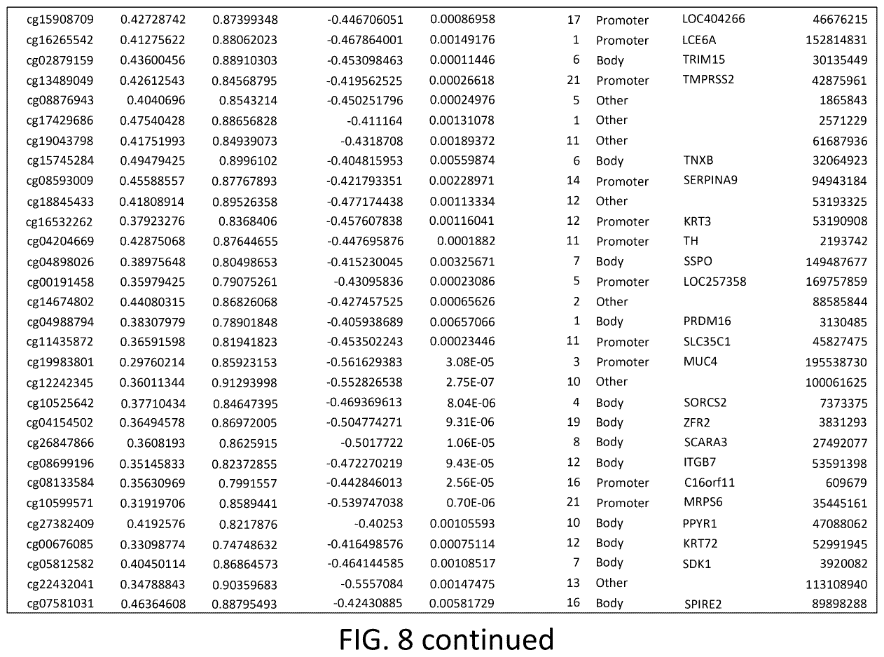

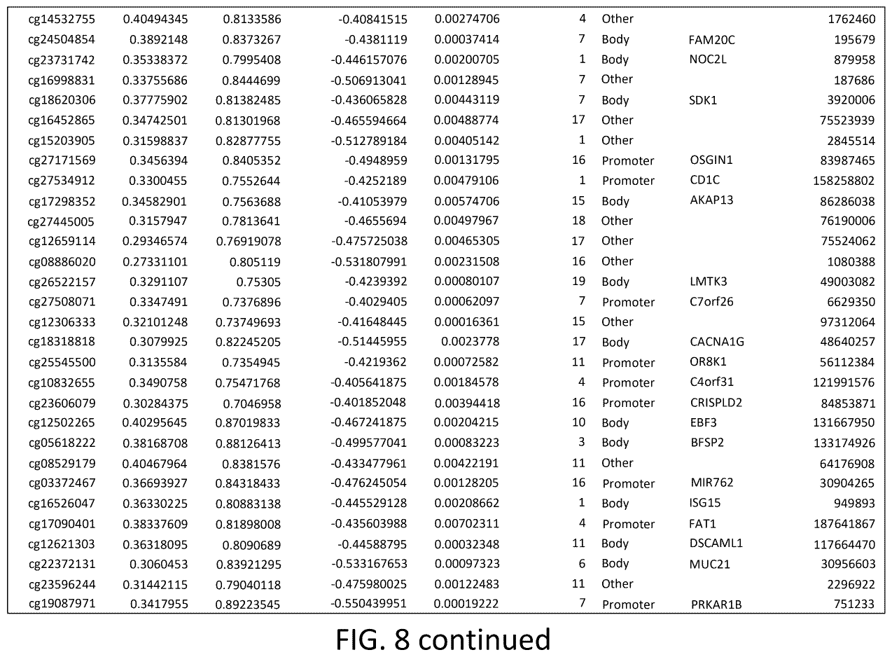

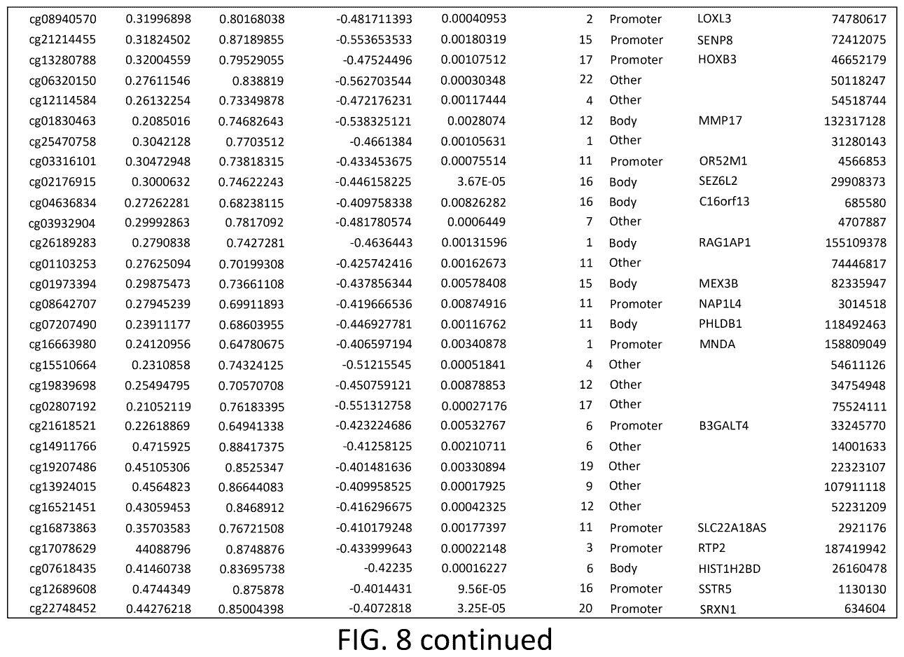

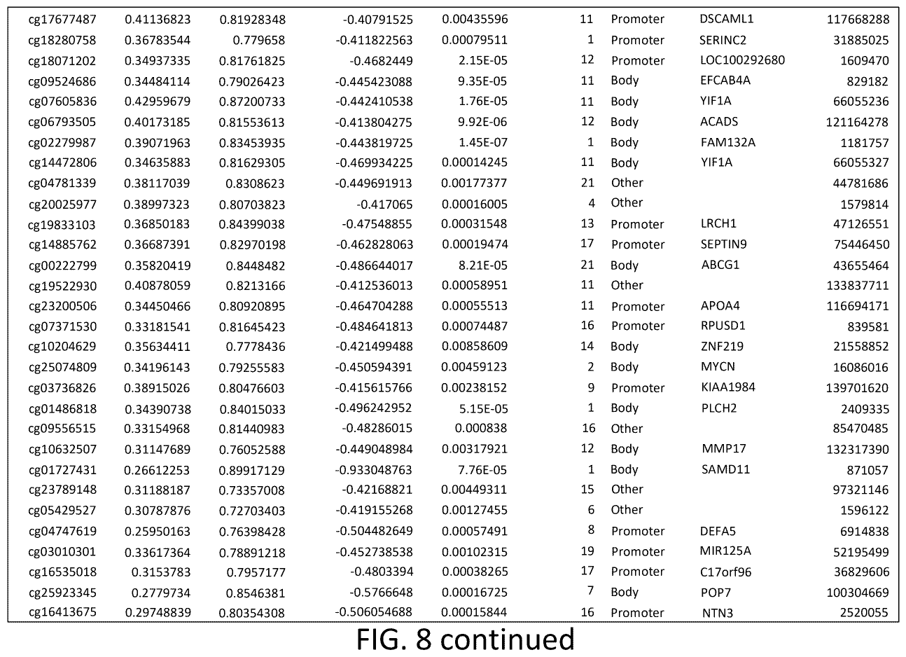

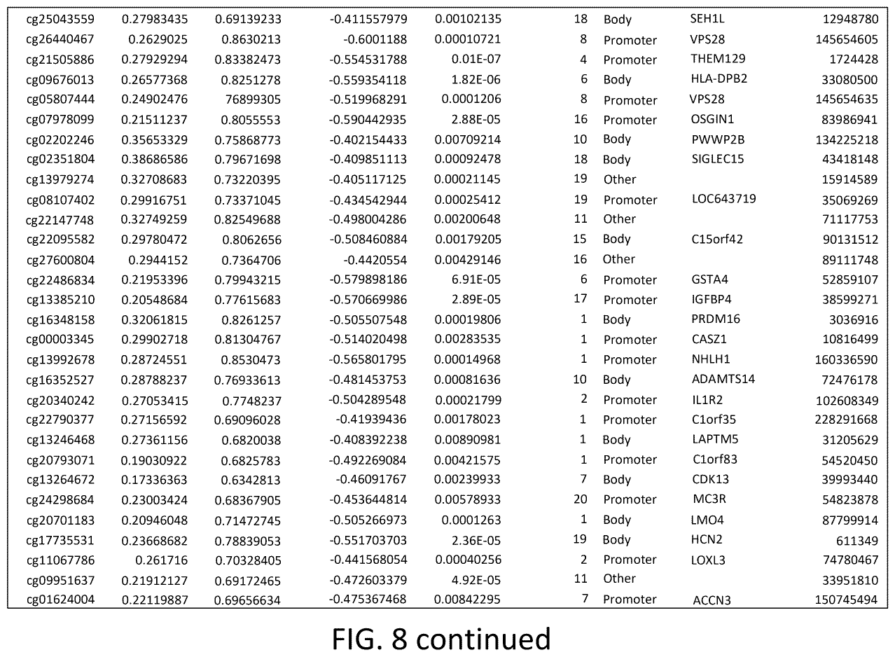

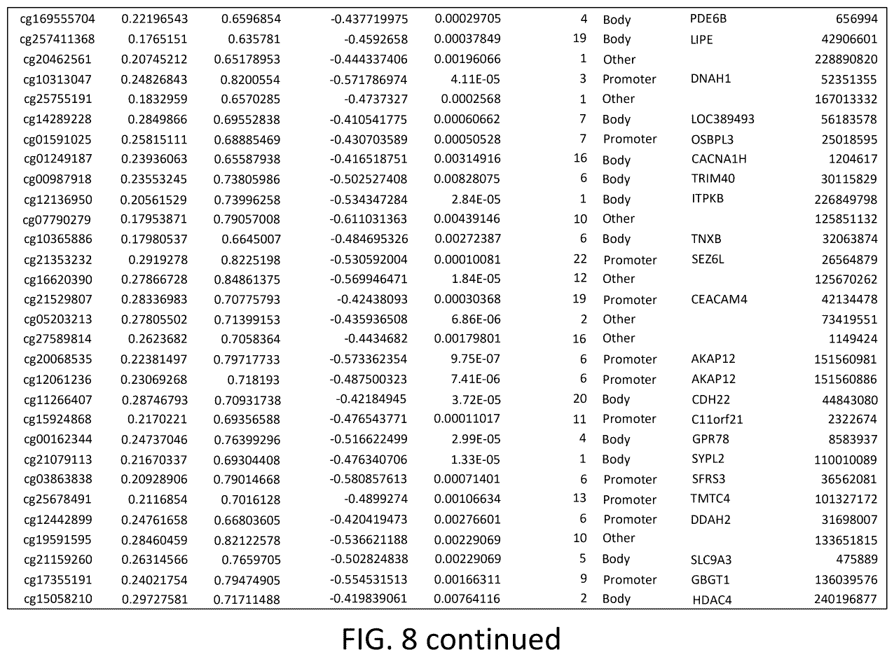

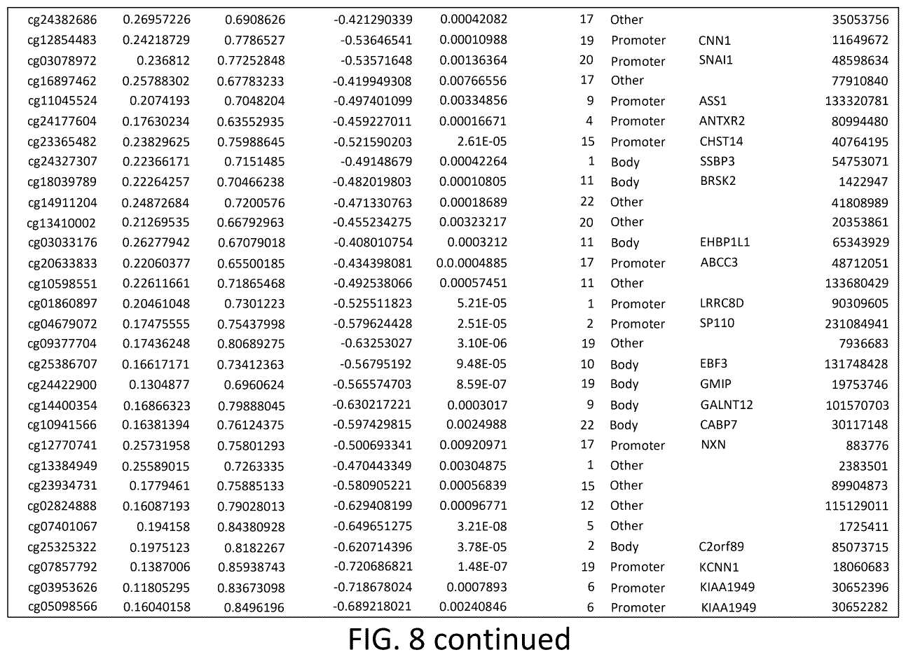

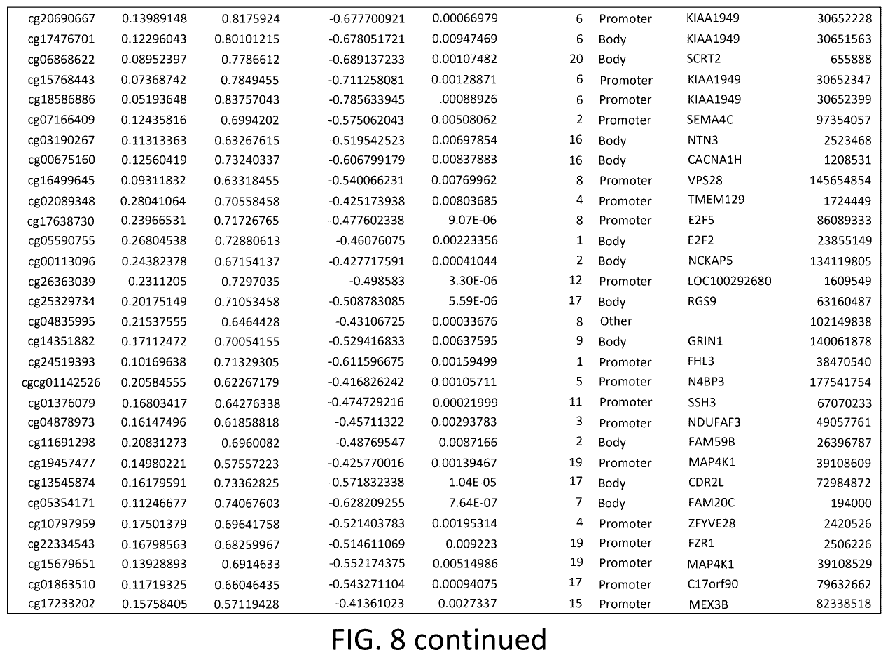

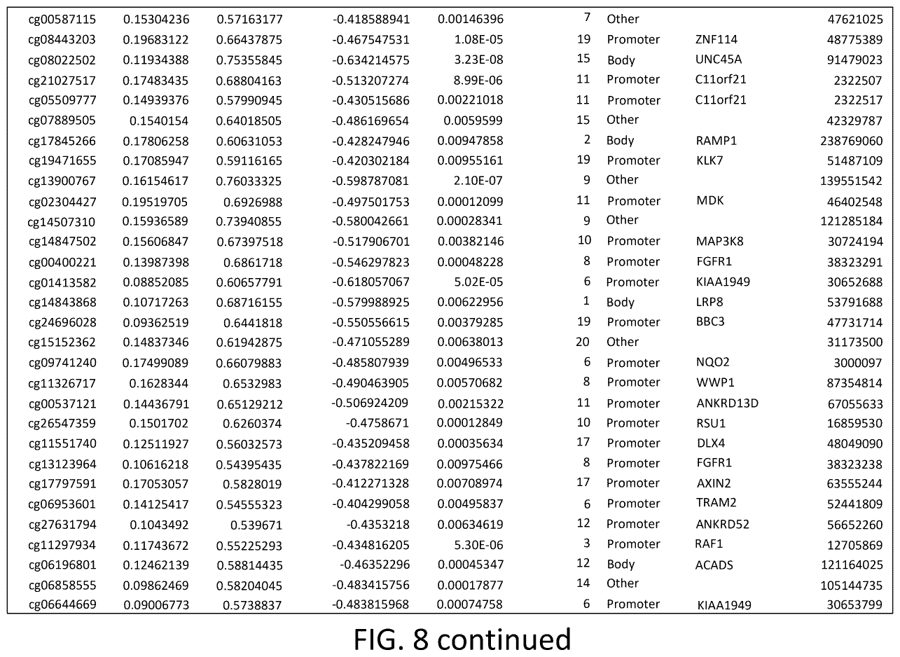

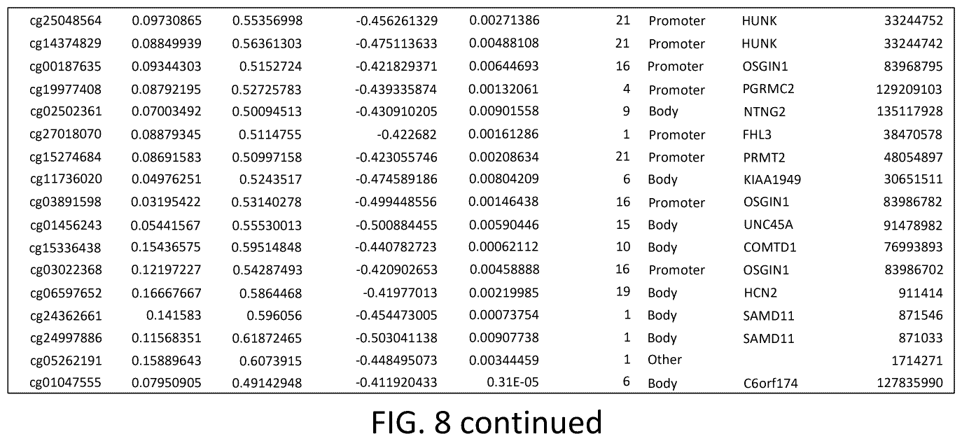

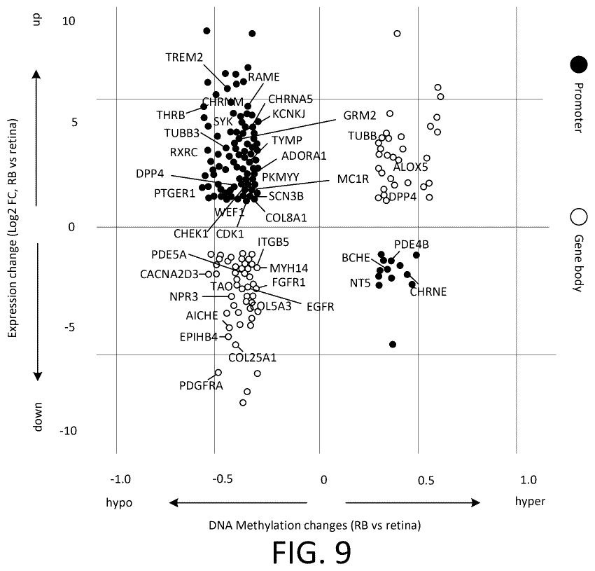

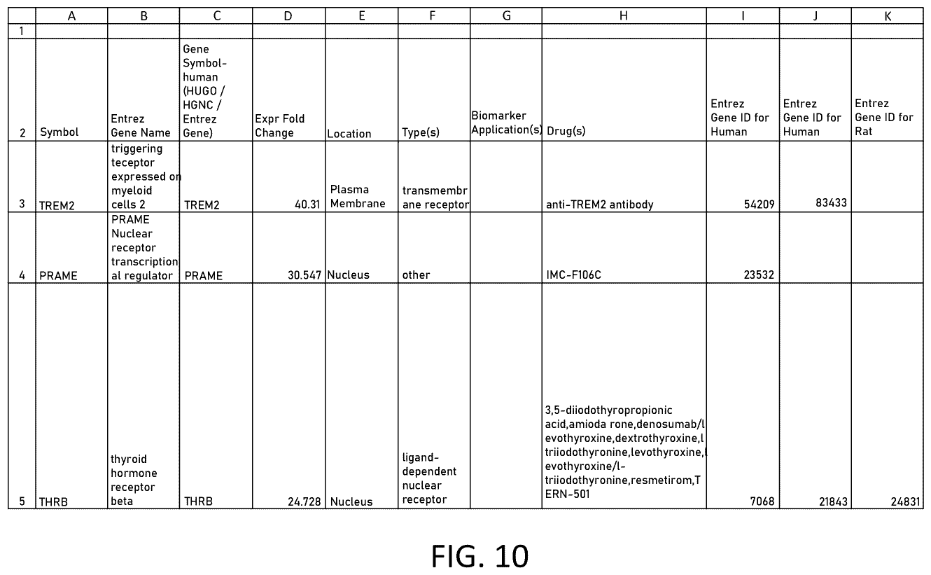

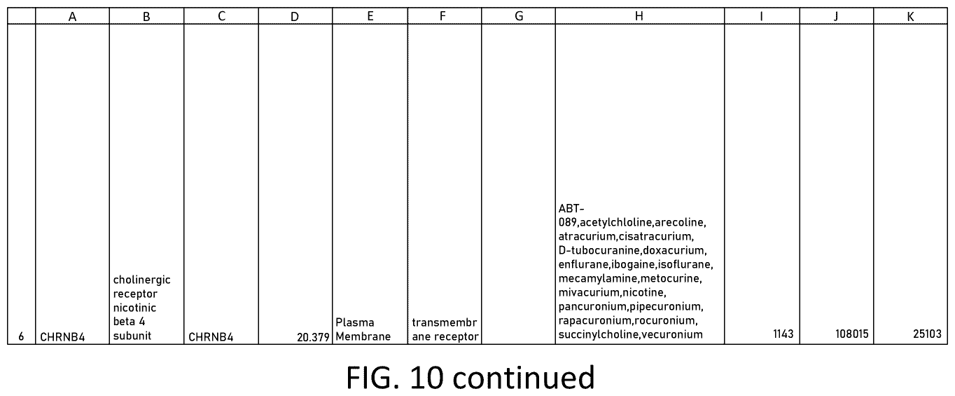

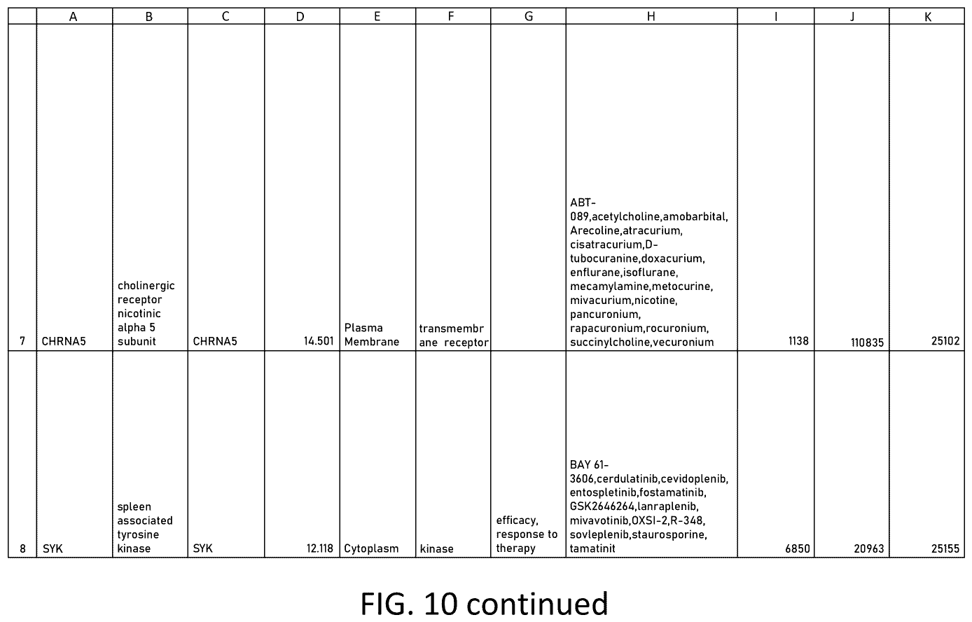

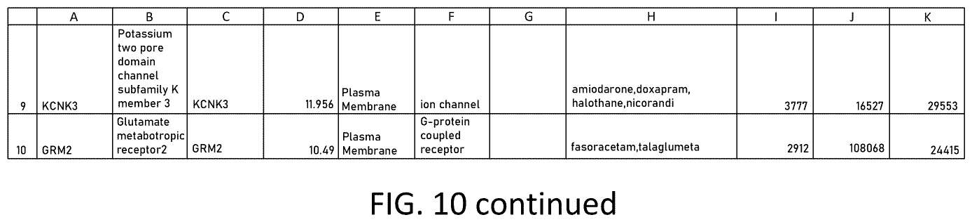

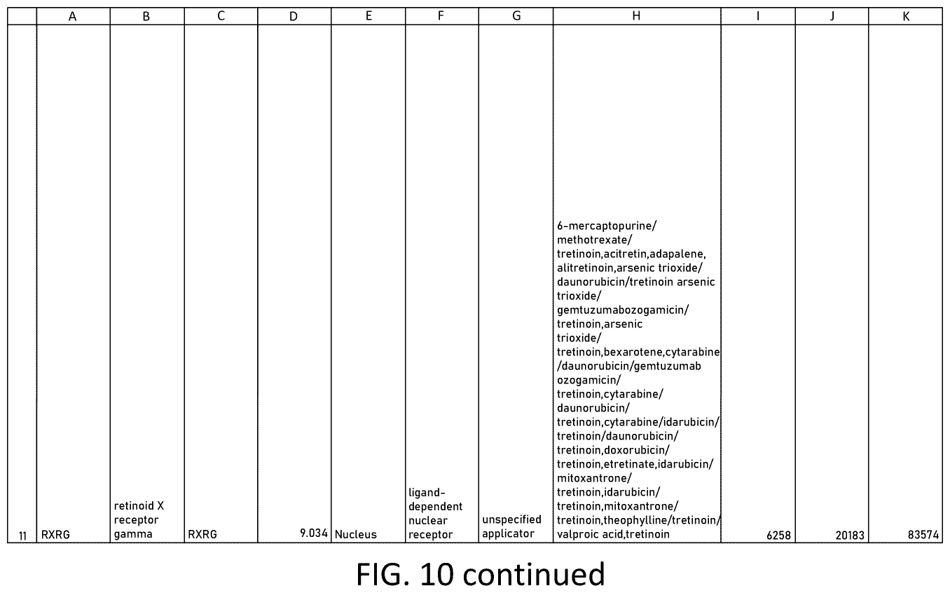

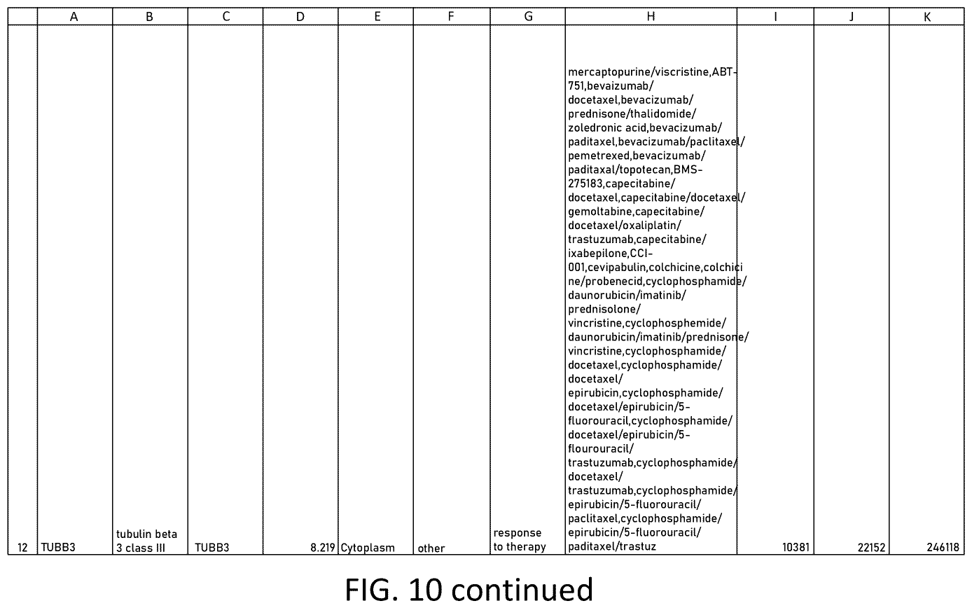

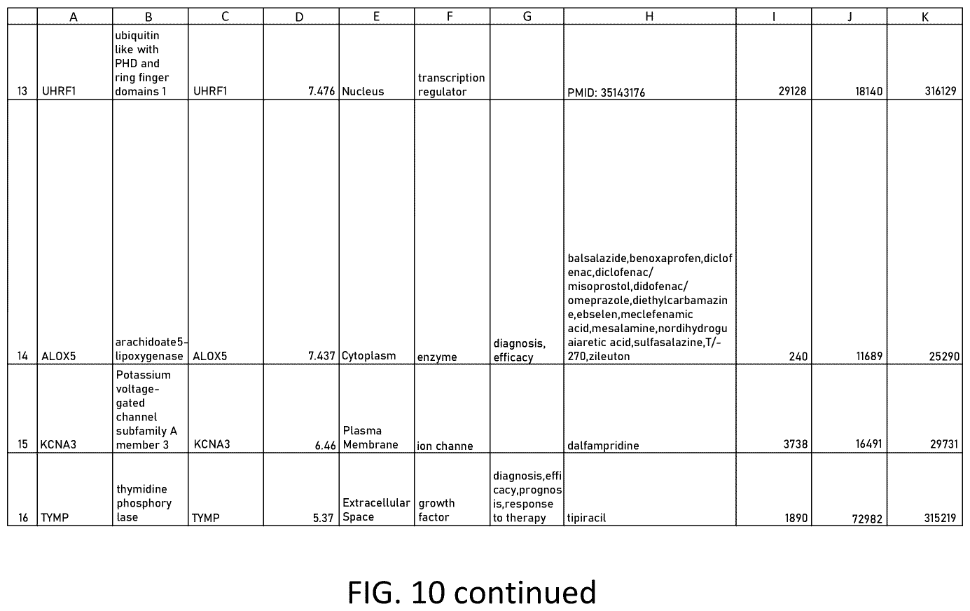

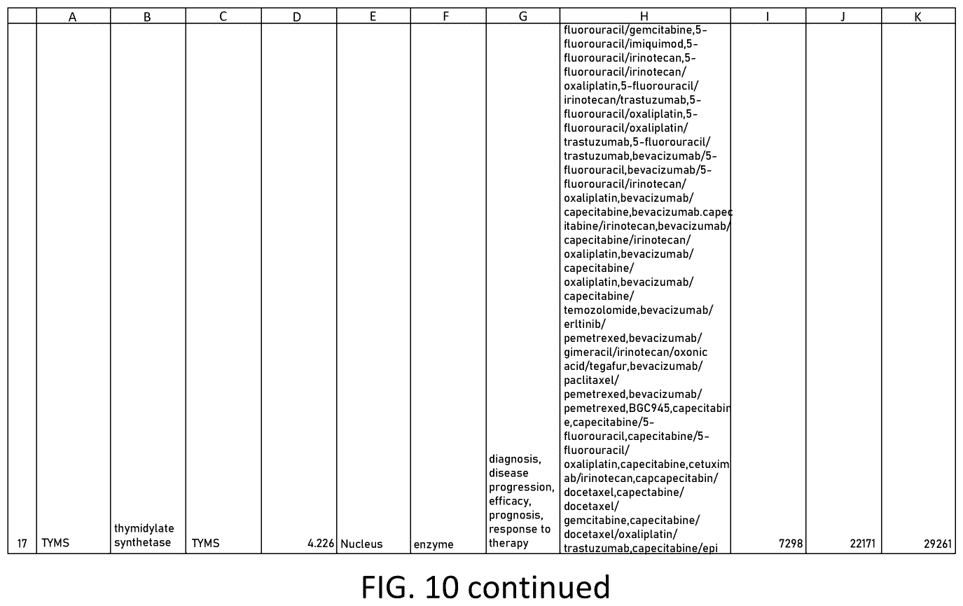

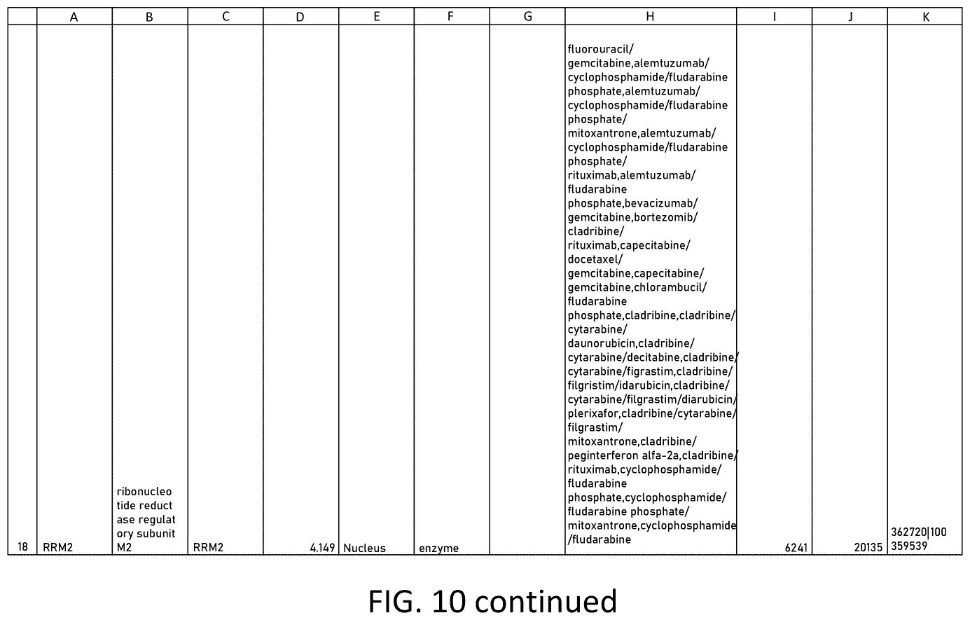

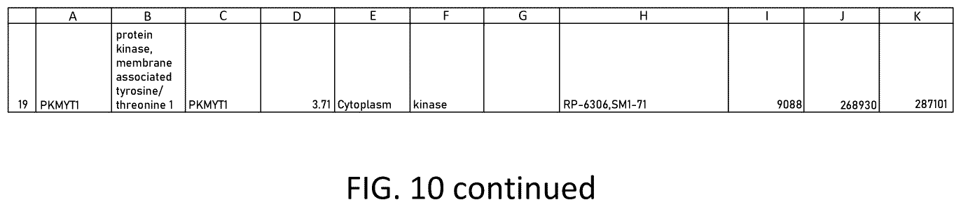

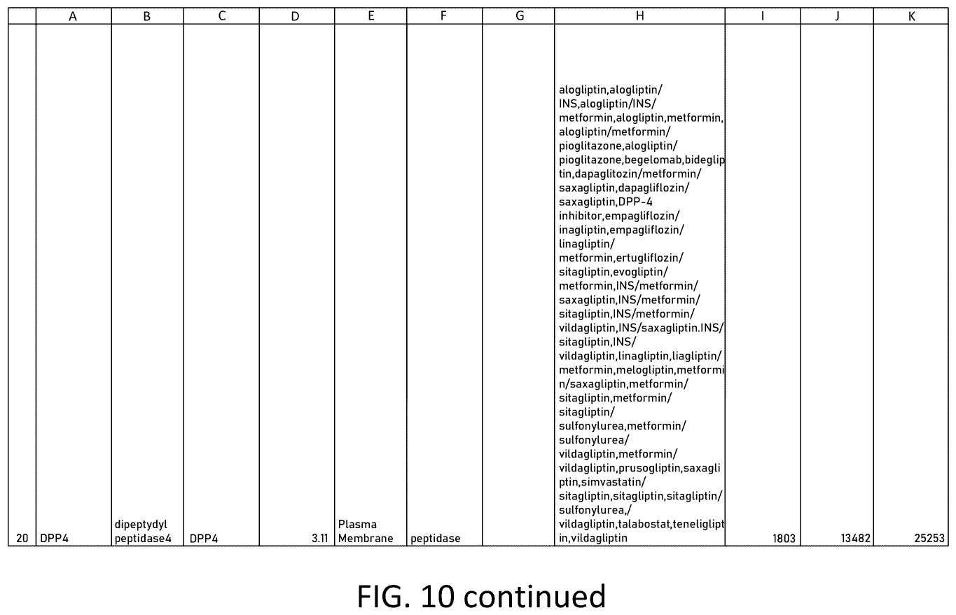

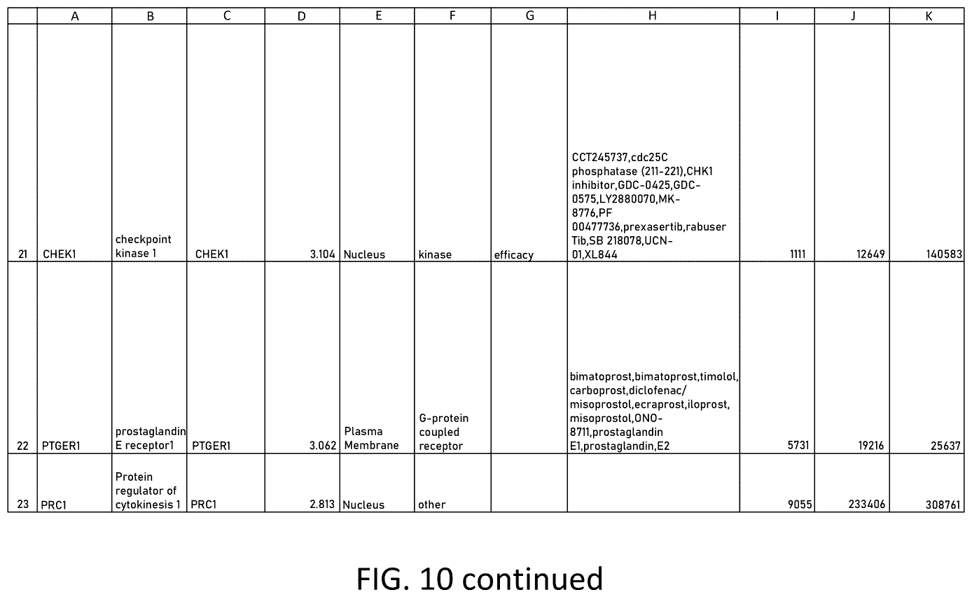

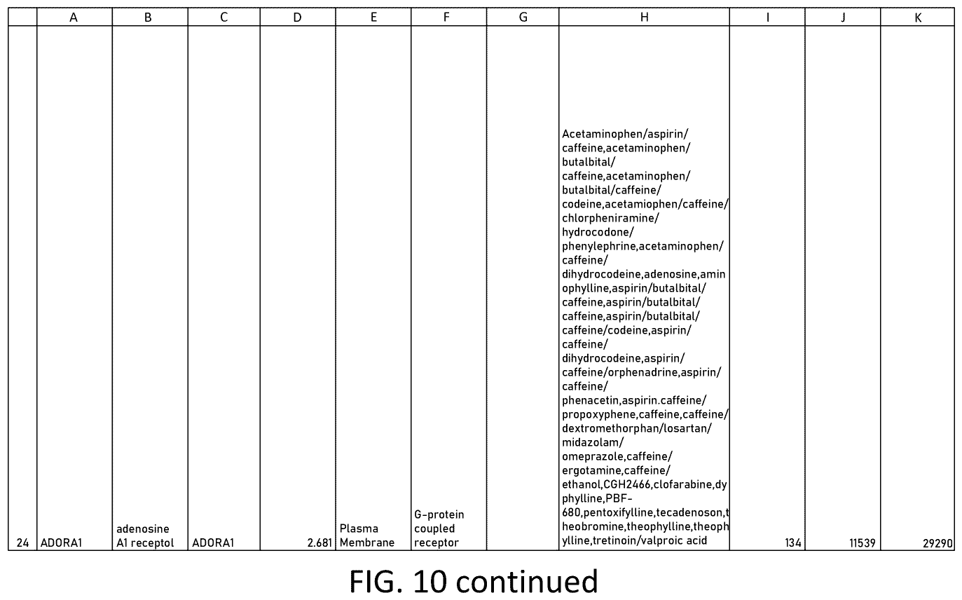

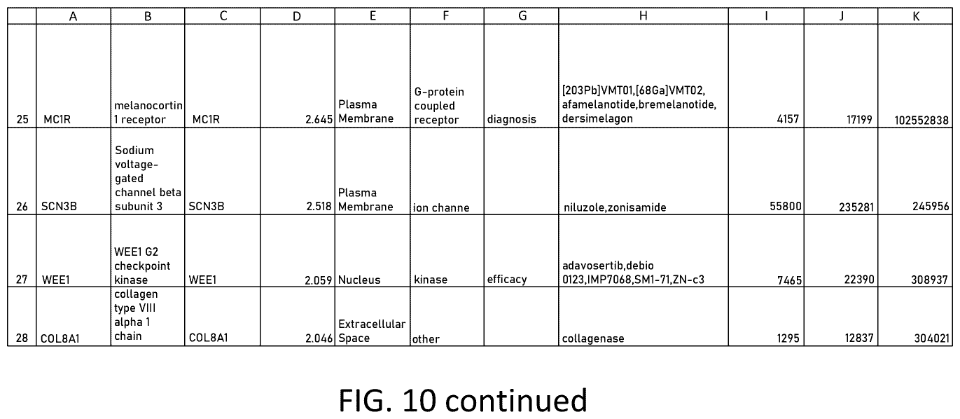

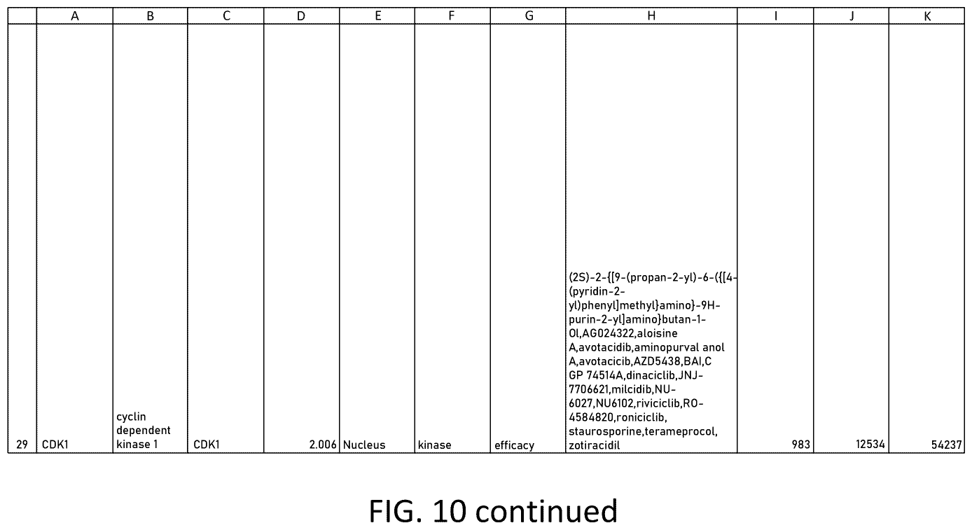

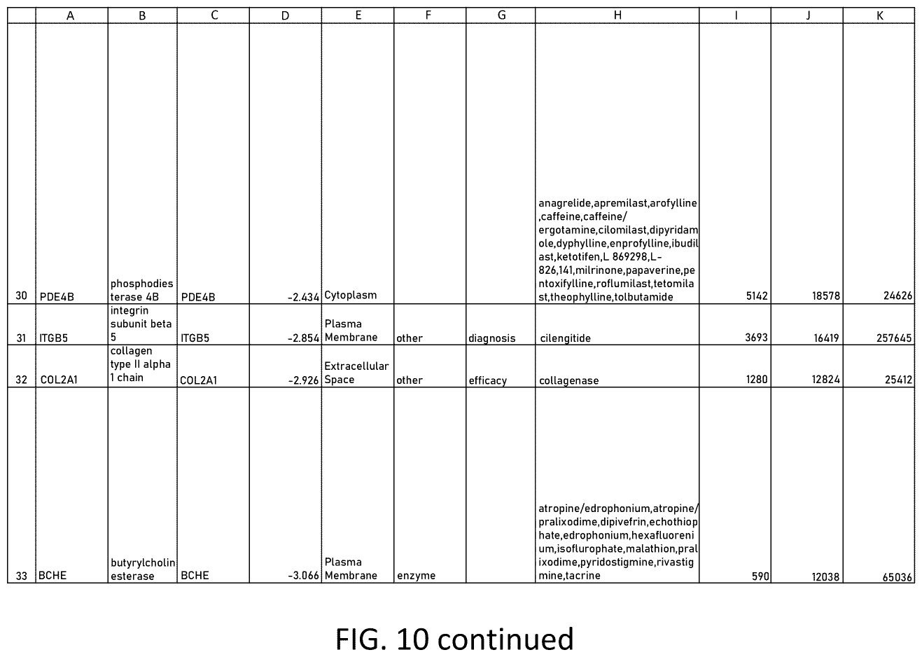

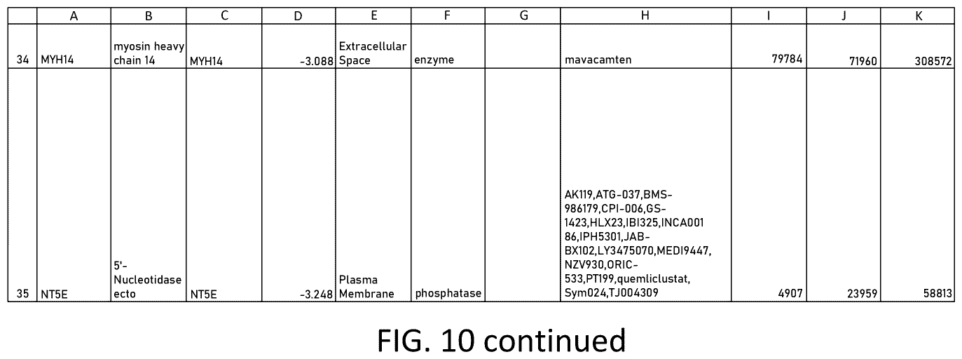

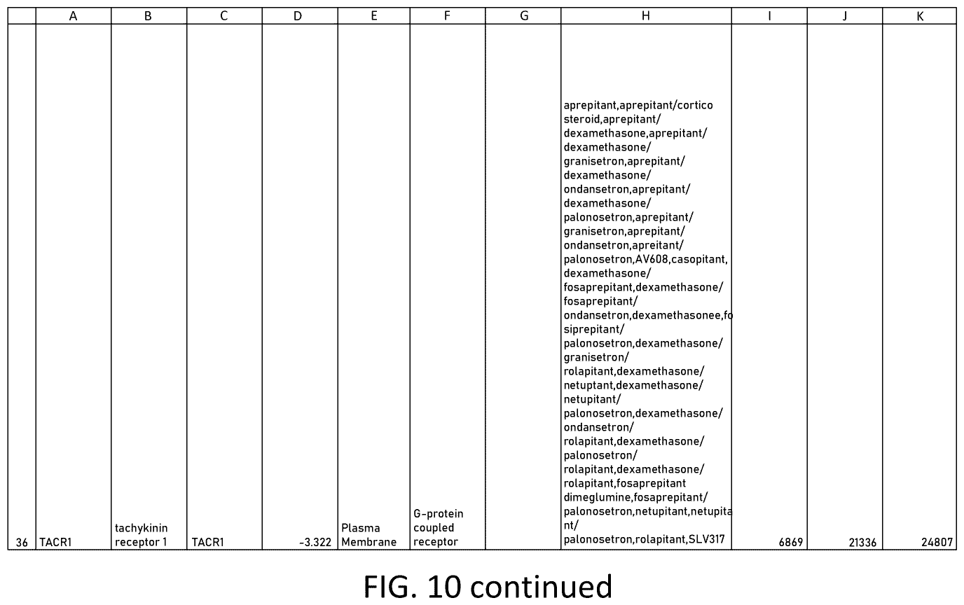

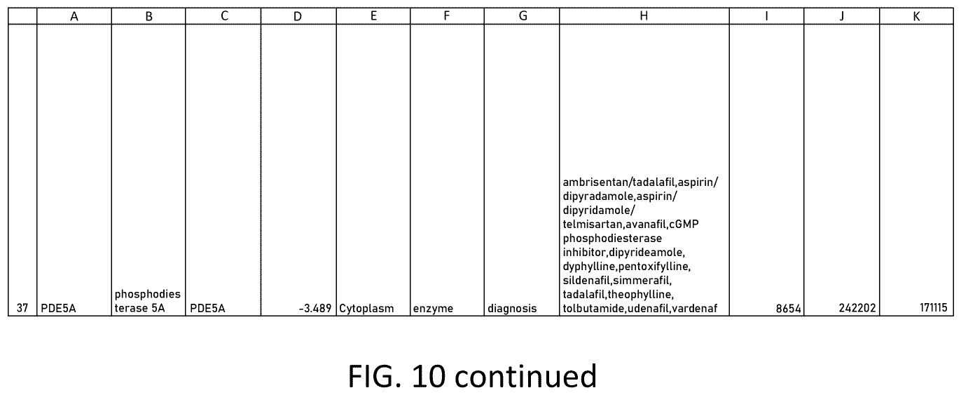

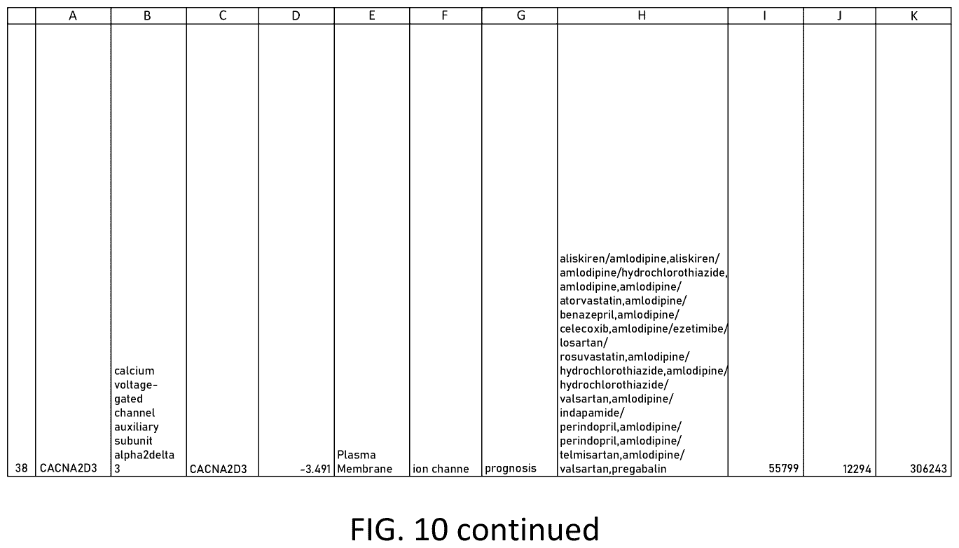

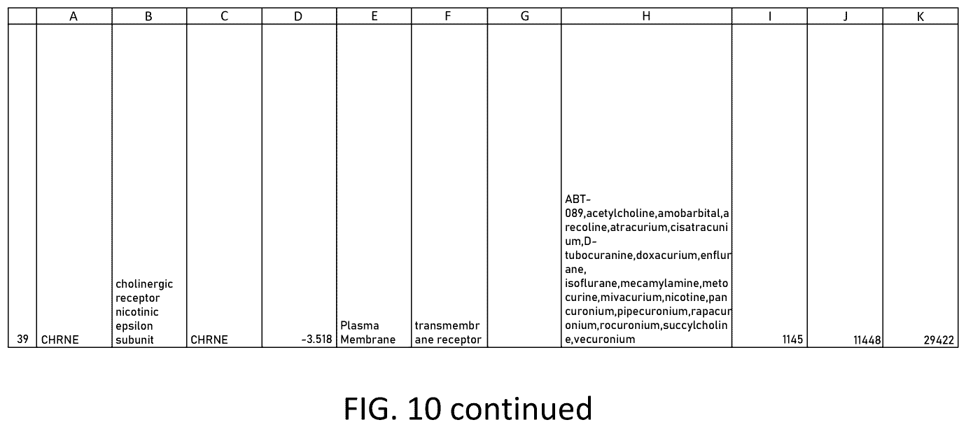

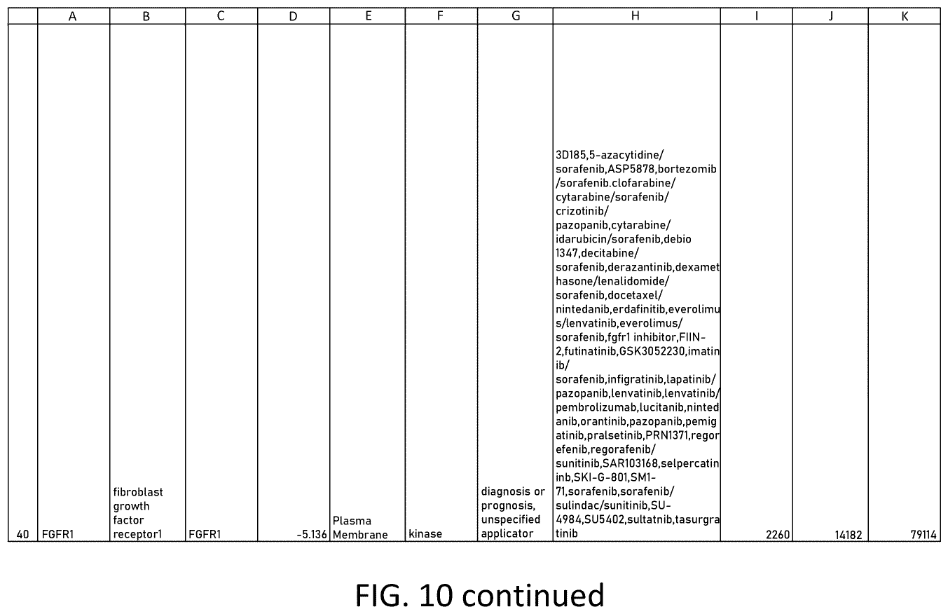

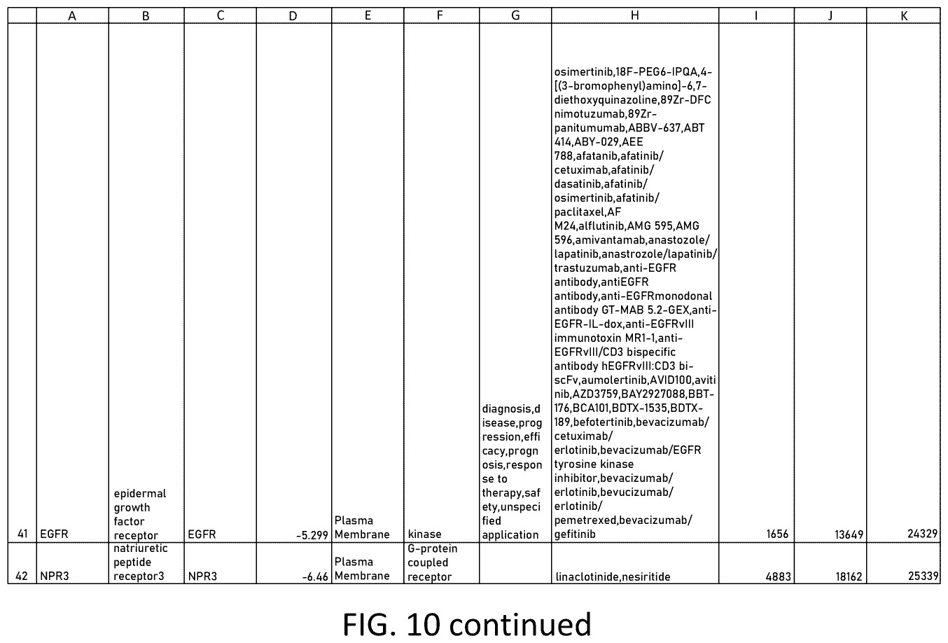

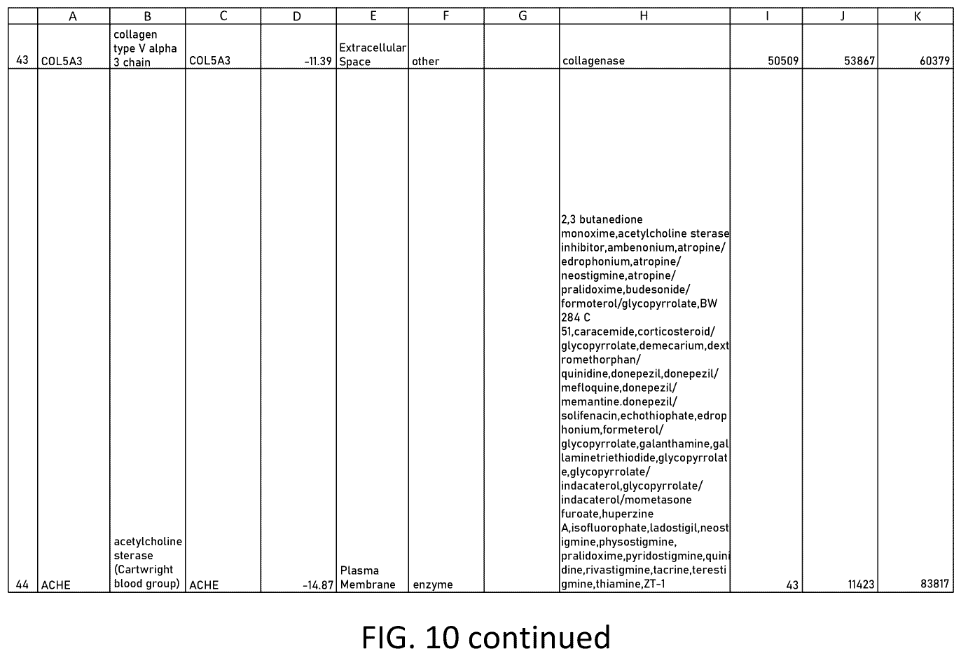

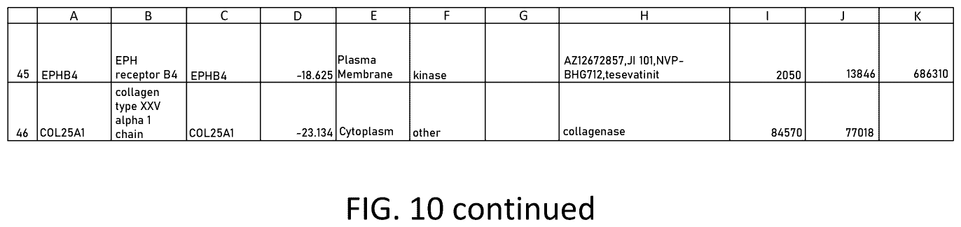

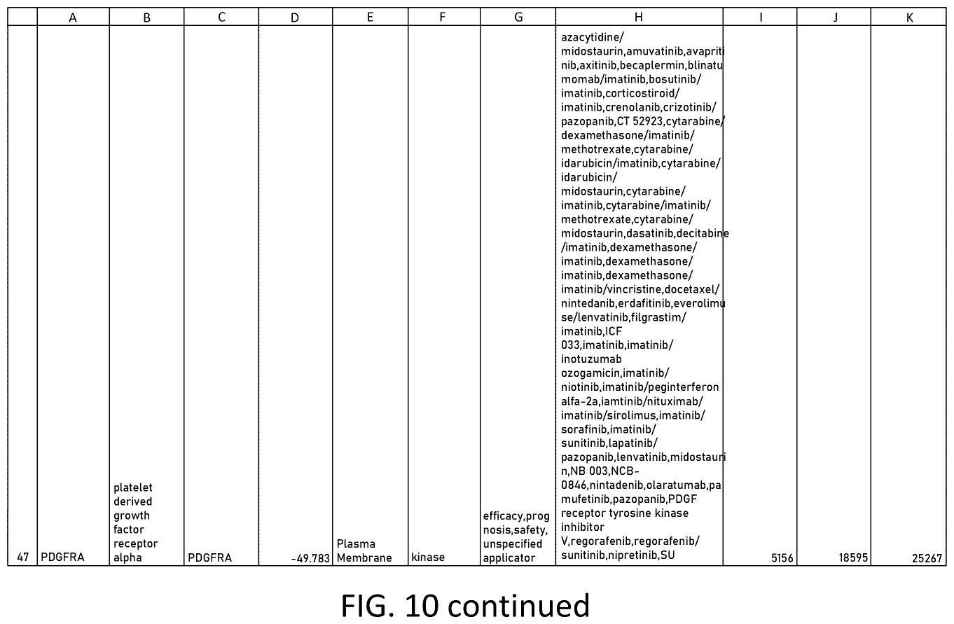

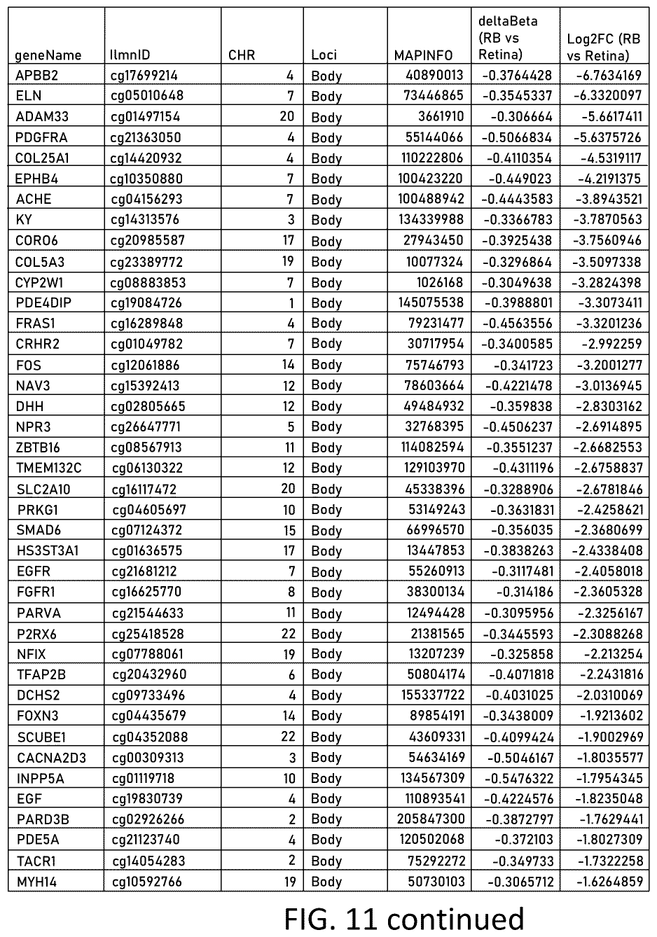

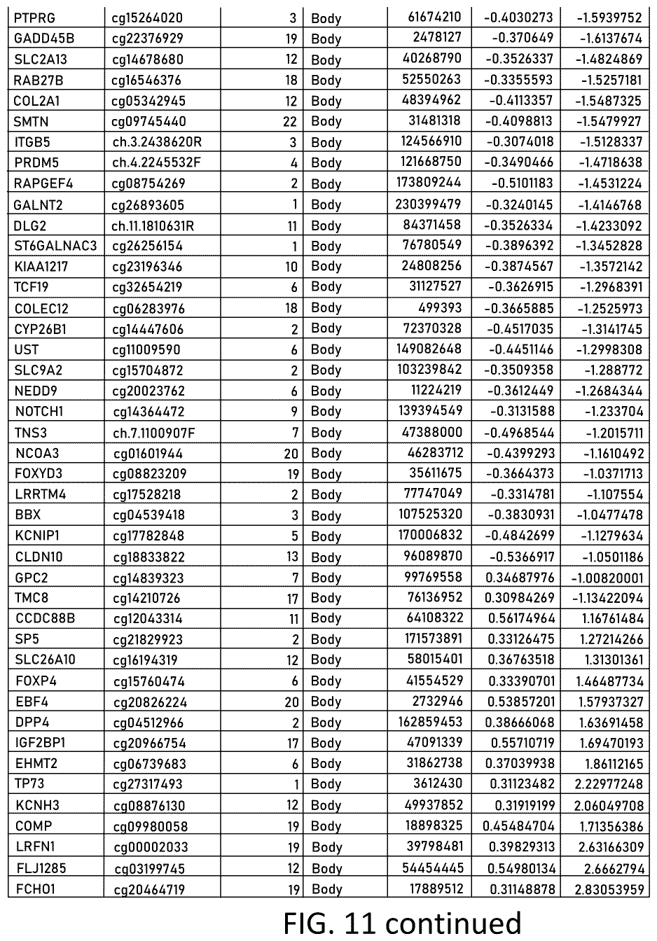

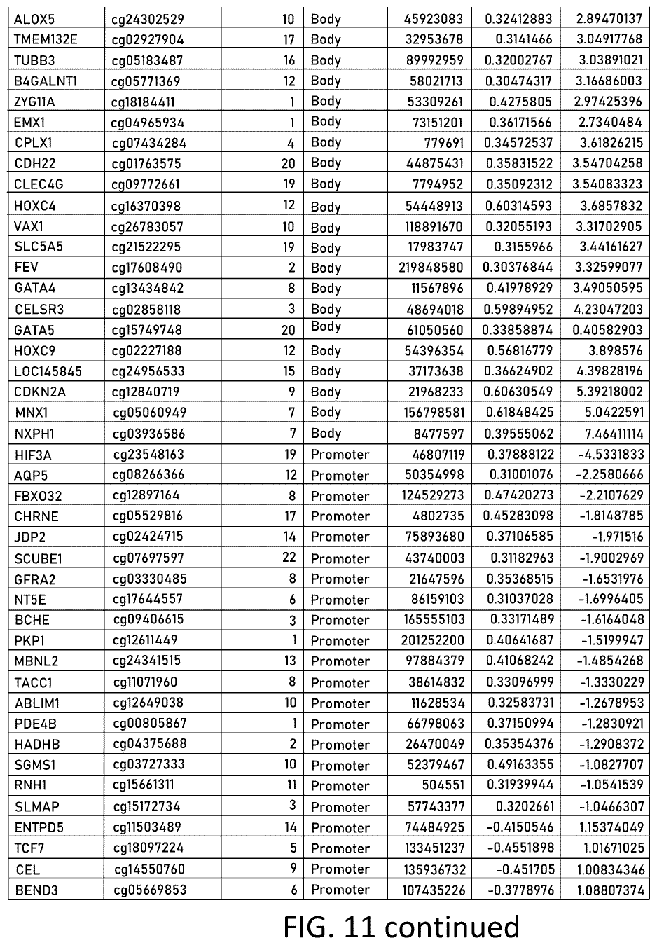

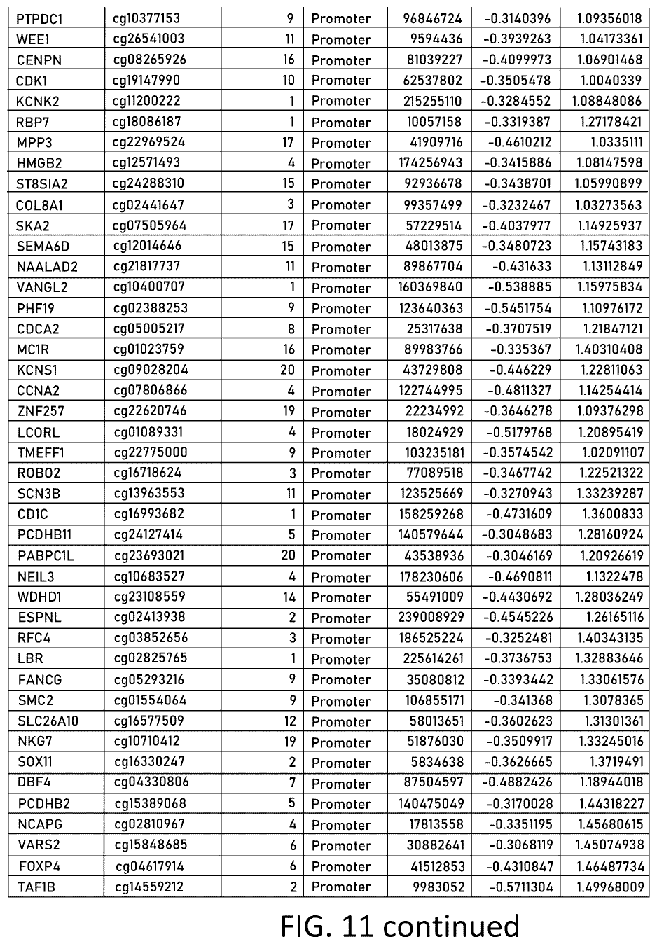

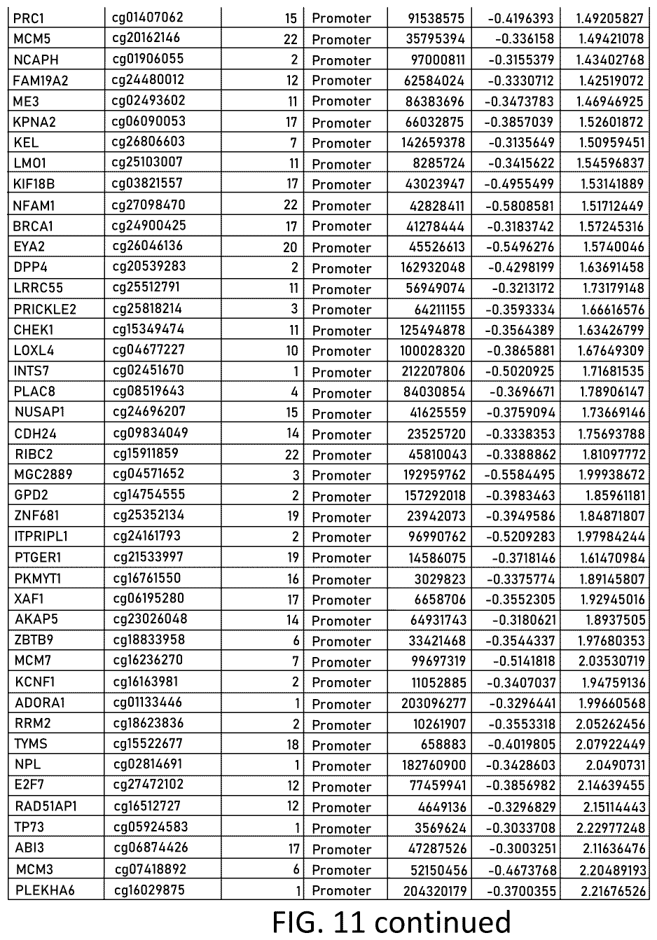

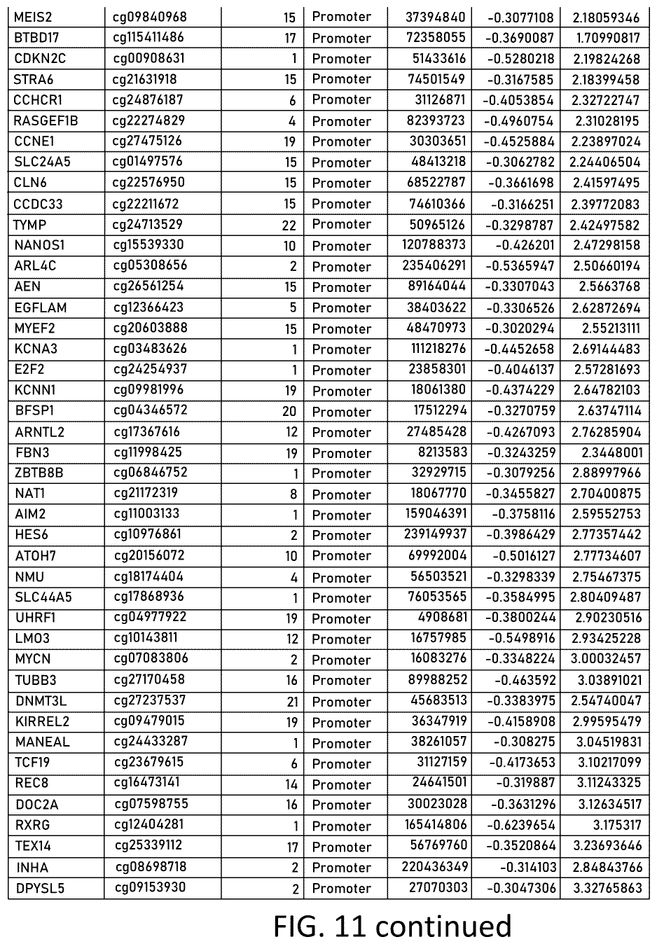

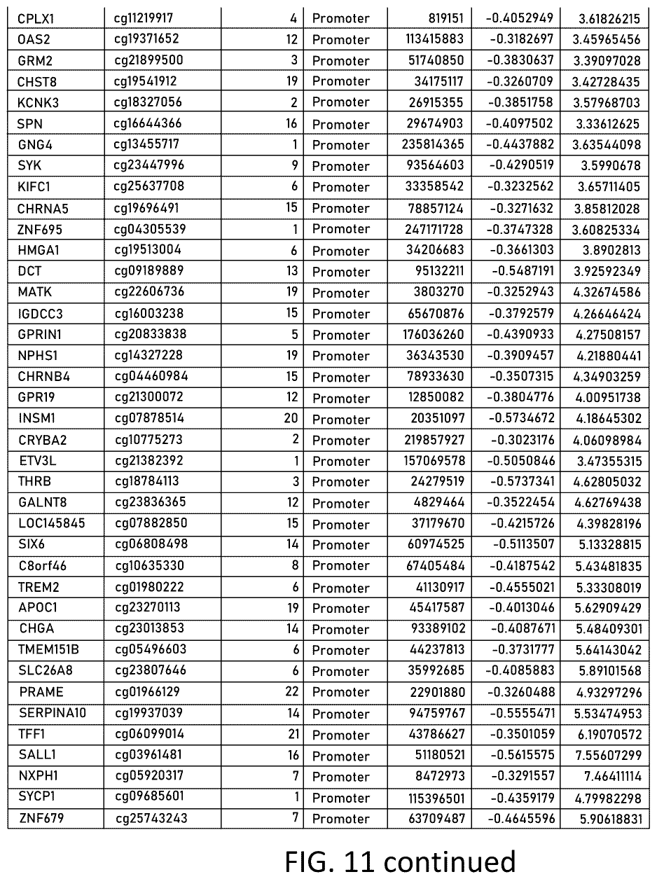

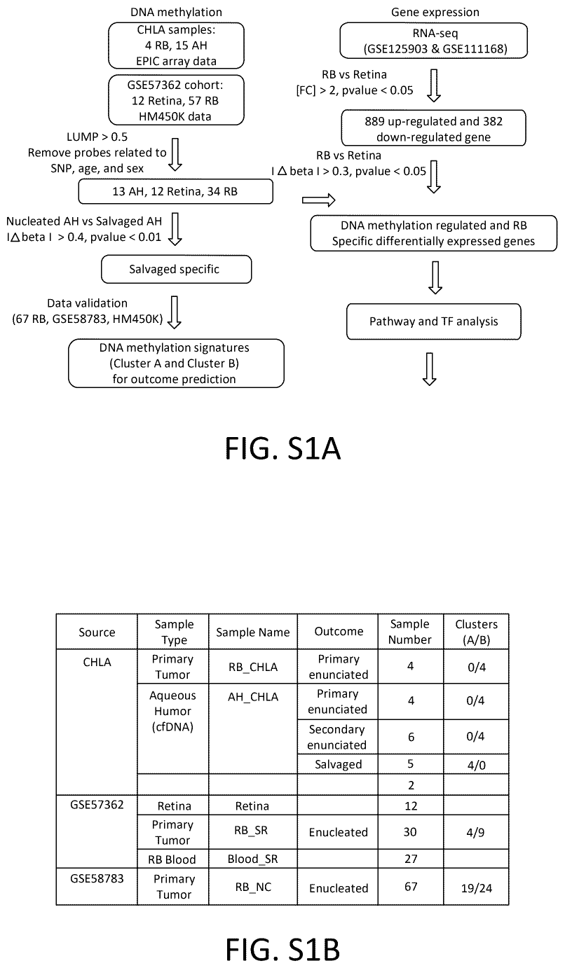

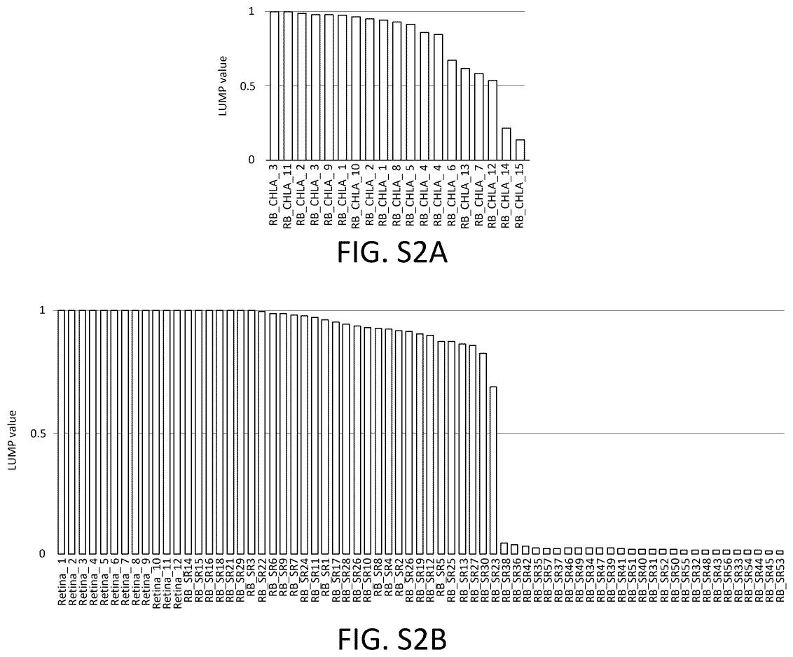

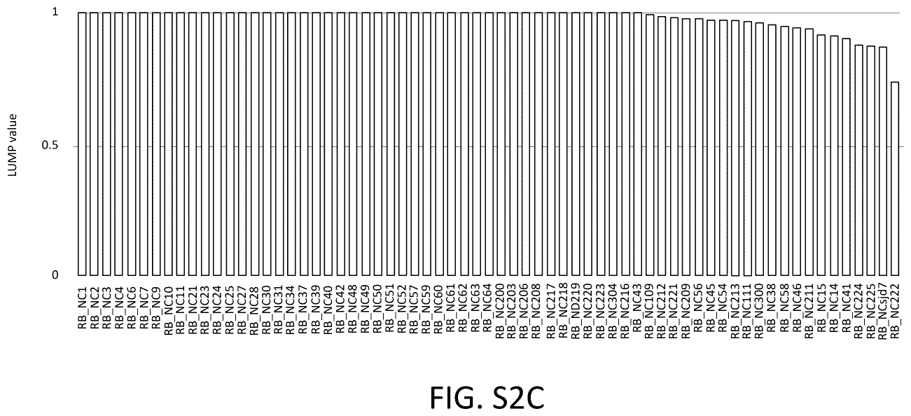

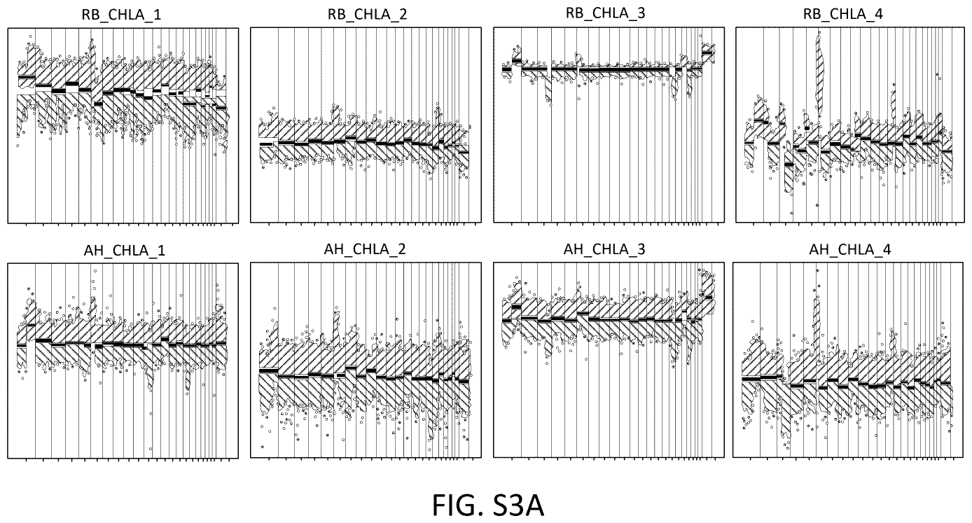

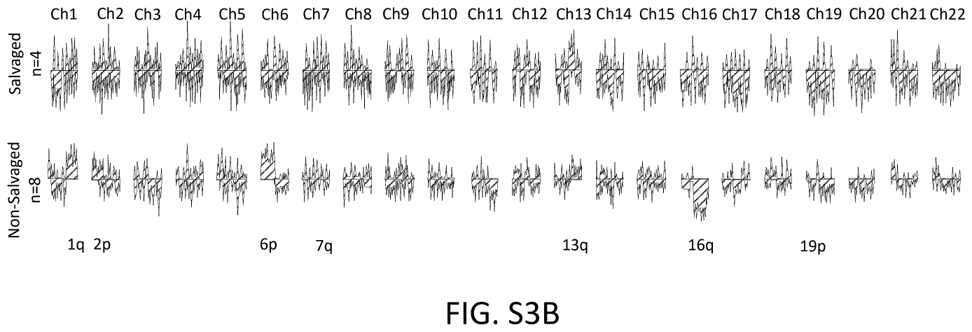

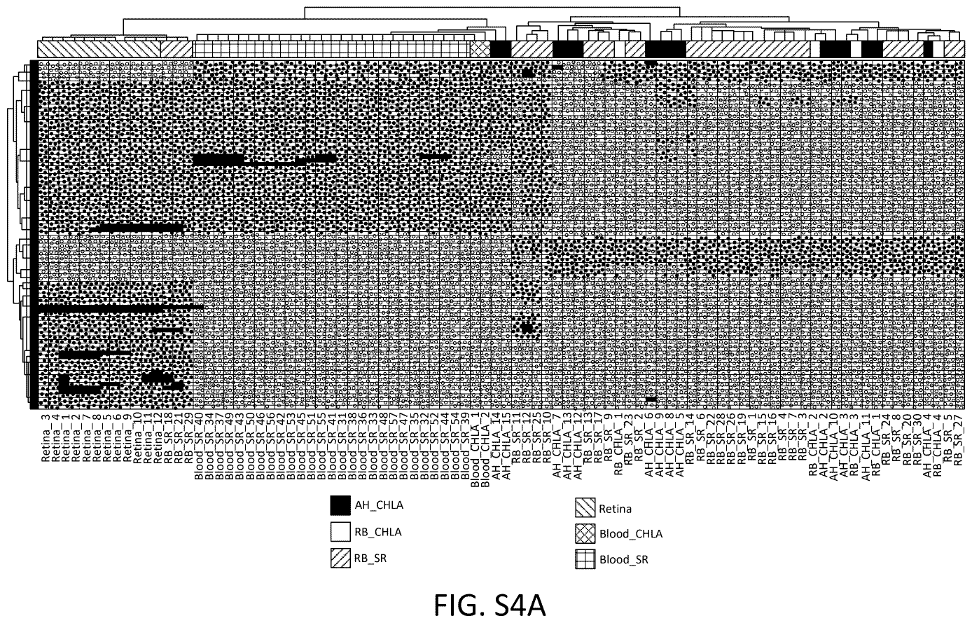

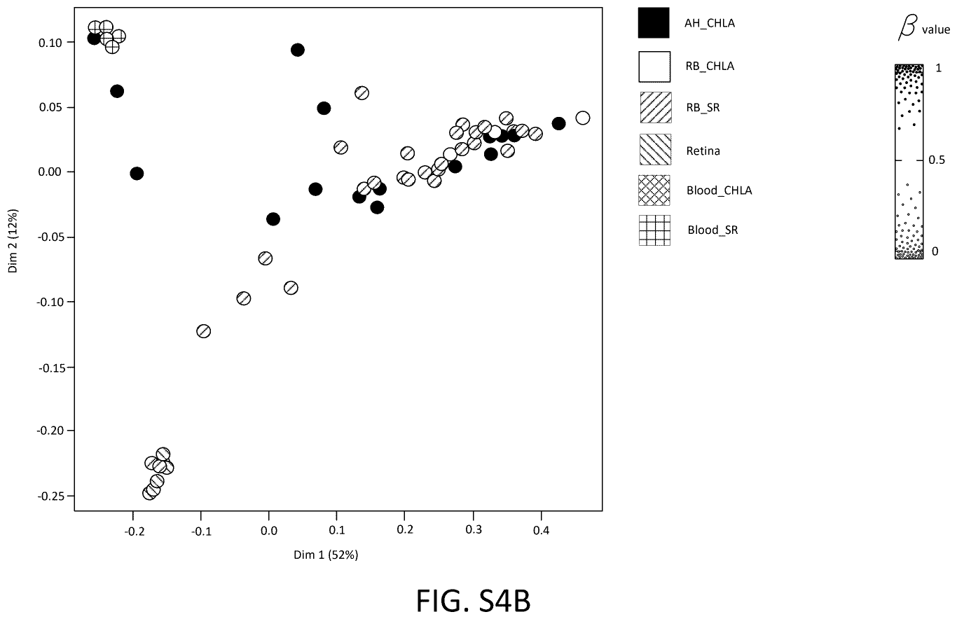

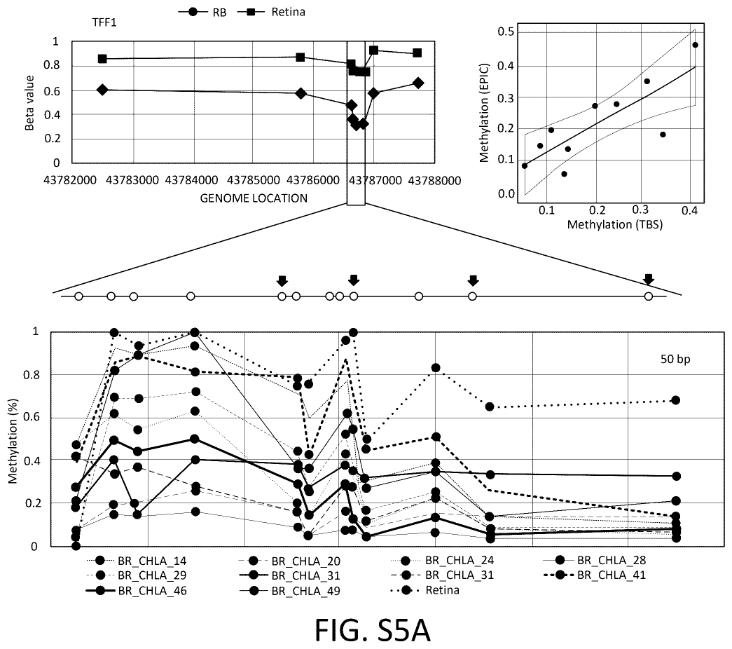

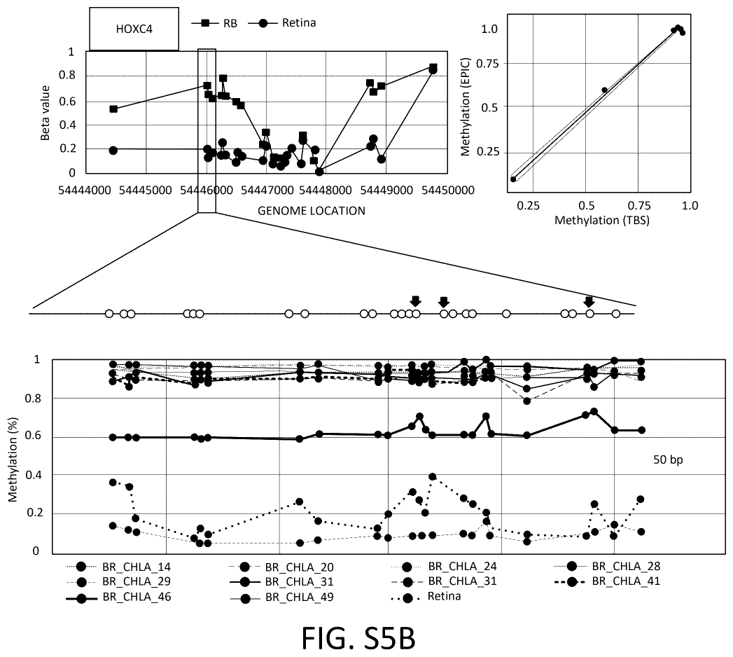

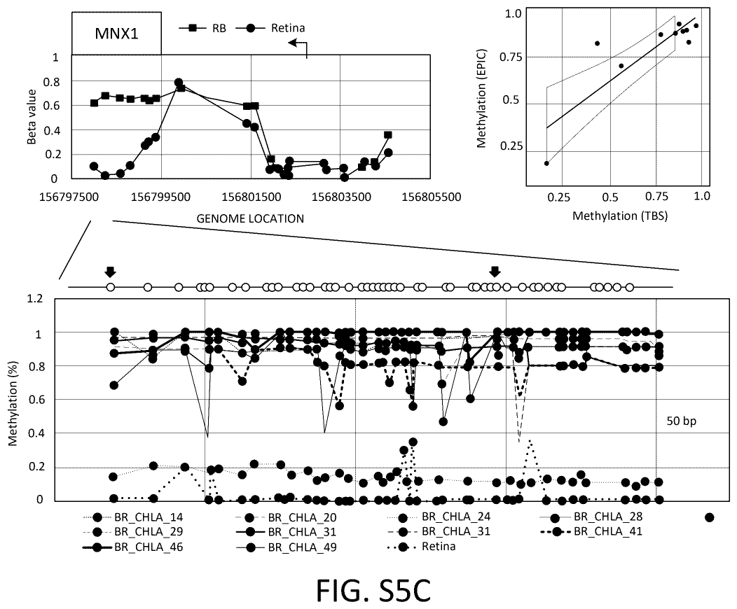

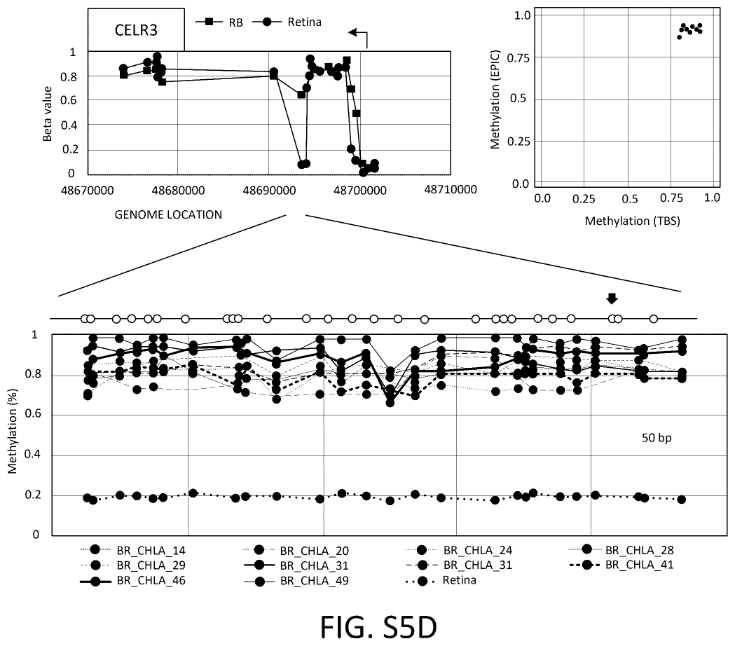

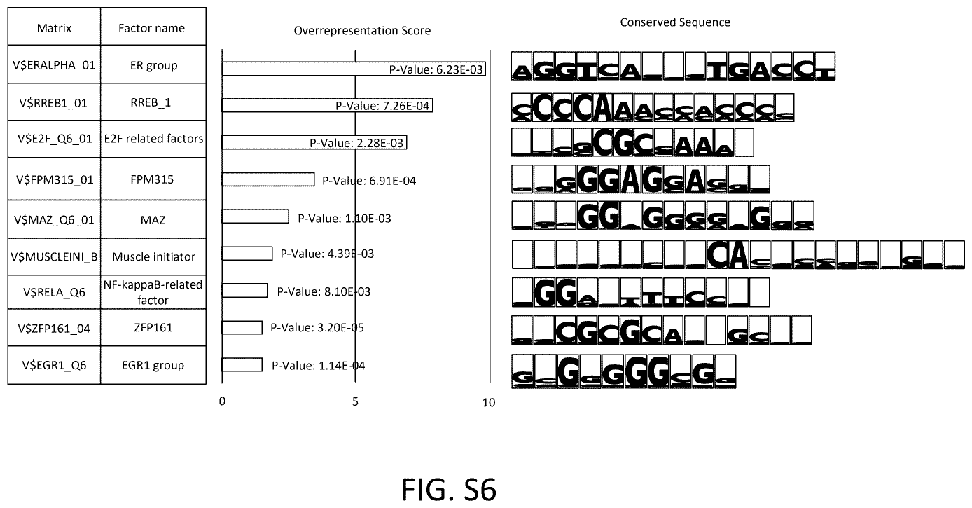

The present invention relates to the use of biomarkers to detect or predict RB. More specifically, the biomarkers of the present invention can be used in diagnostic tests to determine, qualify, and/or assess RB status, for example, to diagnose or predict RB, in an individual, subject or patient. More specifically, the biomarkers to be detected in diagnosing RB include, but are not limited to those provided in FIGS. 7, 8m 10 and 11. These gene sequences are publicly available, as well as their coding sequence/mRNA sequences).

Exemplary human gene sequences include: TFF1 (trefoil factor 1; NC_000021.9 Chromosome 21 Reference GRCh38.p14 (GCF_000001405.40)); GSTA4 (glutathione S-transferase alpha 4; NC_000006.12 Chromosome 6 Reference GRCh38.p14 (GCF_000001405.40)); AXIN2 (axin-2; NG_012142; NC_000017.11 Chromosome 17 Reference GRCh38.p14 (GCF_000001405.40); STK19 (serine/threonine kinase 19; NC_000006.12 Chromosome 6 Reference GRCh38.p14 (GCF_000001405.40)); FGFR1 (fibroblast growth factor receptor 1; NCBI Reference Sequence NG_007729; NC_000008.11 Chromosome 8 Reference GRCh38.p14 (GCF_000001405.40)); MAP4K1 (mitogen-activated protein kinase kinase kinase kinase 1; NC_000019.10 Chromosome 19 Reference GRCh38.p14 (GCF_000001405.40)); FZR1 (fizzy and cell division cycle 20 related 1; NC_000019.10 Chromosome 19 Reference GRCh38.p14 (GCF_000001405.40)); IL1R2 (interleukin 1 receptor type 2; NC_000002.12 Chromosome 2 Refence GRCh38.p14 (GCF_000001405.40)); SORBS2 (sorbin and SH3 domain containing 2; NCBI Reference Sequence: NG_029709.1; NC_000004.12 Chromosome 4 Reference GRCh38.p14 (GCF_000001405.40)); DDC (DCC netrin 1 receptor; NCBI Reference Sequence: NG_013341.2 NC_000018.10 Chromosome 18 Reference GRCh38.p14 (GCF_000001405.40)); MPP7 (MAGUK p55 scaffold protein 7; NC_0000010.11 Chromosome 10 Reference GRCh38.p14 (GCF_000001405.40)); PTGS2 (prostaglandin-endoperoxide synthase 2; NCBI Reference Sequence: NG_028206.2; NC_000001.11 Chromosome 1 Reference GRCh38.p14 (GCF_000001405.40)); PSMD1 (proteasome 26S subunit, non-ATPase 1; NC 000002.12 Chromosome 2 Reference GRCh38.p14 (GCF_000001405.40)); GUCA1C (guanylate cyclase activator 1C, NC_000003.12 Chromosome 3 Reference GRCh38.p14 (GCF_000001405.40)); CAB39L (calcium binding protein 39 like; NC_000013.11 Chromosome 13 Reference GRCh38.p14 (GCF_000001405.40)); AKAP13 (A-kinase anchoring protein 13; GenBank: LS482295.1; NC_000015.10 Chromosome 15 Reference GRCh38.p14 (GCF_000001405.40)); MYCN (MYCN proto-oncogene, bHLH transcription factor; NCBI Reference Sequence: NG_007457.2; NC_000002.12 Chromosome 2 Reference GRCh38.p14 (GCF_000001405.40)); SYK (spleen associated tyrosine kinase; NCBI Reference Sequence: NG_017046.2; NC_000009.12 Chromosome 9 Reference GRCh38.914 (GCF_000001405.40)); RB1 (RB transcriptional corepressor 1; NCBI Reference Sequence: NG_009009.1; NC 000013.11 Chromosome 13 Reference GRCh38.p14 (GCF_000001405.40)); and E2F (E2F transcription factor 1; NCBI Reference Sequence: NG_046988.1; NC_000020.11 Chromosome 20 Reference GRCh38.p14 (GCF_000001405.40)).

### A. Biomarker Panels

The biomarkers of the present invention can be used in diagnostic and/or prognostic tests to assess, determine, confirm and/or qualify (used interchangeably herein) RB status in a patient. The phrase “RB status” includes any distinguishable manifestations of the disease, as well as unaffected patients. For example, RB status includes, without limitation, the presence or absence of RB in a patient), the risk of developing RB, the risk of developing aggressive or recurring RB, the stage of RB, the progress of RB (e.g., progress of RB over time) and the effectiveness or response to treatment of RB (e.g., clinical follow up and surveillance of RB after treatment), and treatment responder status. Based on this status, further procedures may be indicated, including additional diagnostic tests or therapeutic procedures or regimens.

The power of a diagnostic test to correctly predict status is commonly measured as the sensitivity of the assay, the specificity of the assay or the area under a receiver operated characteristic (“ROC”) curve. Sensitivity is the percentage of true positives that are predicted by a test to be positive, while specificity is the percentage of true negatives that are predicted by a test to be negative. An ROC curve provides the sensitivity of a test as a function of 1-specificity. The greater the area under the ROC curve, the more powerful the predictive value of the test. Other useful measures of the utility of a test are positive predictive value and negative predictive value. Positive predictive value is the percentage of people who test positive that are actually positive. Negative predictive value is the percentage of people who test negative that are actually negative.

In some embodiments, the biomarker panels of the present invention may show a statistical difference in different RB statuses of at least p<0.05, p<10-2, p<10-3, p<10-4 or p<10-5. Diagnostic tests that use these biomarkers may show an ROC of at least 0.6, at least about 0.7, at least about 0.8, or at least about 0.9.

The biomarkers are differentially methylated in unaffected and RB, and, therefore, are useful in aiding in the determination of RB status. In certain embodiments, the biomarkers are measured in a patient sample using the methods described herein and compared, for example, to predefined biomarker levels and correlated to RB status. In particular embodiments, the measurement(s) may then be compared with a relevant diagnostic amount(s), cut-off(s), or multivariate model scores that distinguish a positive RB status from a negative RB status. The diagnostic amount(s) represents a measured amount of a hypermethylated or hypomethylated biomarker(s) above which or below which a patient is classified as having a particular RB. For example, if the biomarker(s) is/are hypermethylated compared to normal during RB, then a measured amount(s) above the diagnostic cutoff(s) provides a diagnosis of RB. Alternatively, if the biomarker(s) is/are hypomethylated in a patient, then a measured amount(s) at or below the diagnostic cutoff(s) provides a diagnosis of RB. As is well understood in the art, by adjusting the particular diagnostic cut-off(s) used in an assay, one can increase sensitivity or specificity of the diagnostic assay depending on the preference of the diagnostician. In particular embodiments, the particular diagnostic cut-off can be determined, for example, by measuring the amount of biomarker hypermethylation in a statistically significant number of samples from patients with the RB statuses and drawing the cut-off to suit the desired levels of specificity and sensitivity.

Indeed, as the skilled artisan will appreciate there are many ways to use the measurements of the methylation status of two or more biomarkers in order to improve the diagnostic question under investigation. In a quite simple, but nonetheless often effective approach, a positive result is assumed if a sample is hypermethylation positive or hypomethylation positive for at least one of the markers investigated.

Furthermore, in certain embodiments, the methylation values measured for markers of a biomarker panel are mathematically combined and the combined value is correlated to the underlying diagnostic question. Methylated biomarker values may be combined by any appropriate state of the art mathematical method. Well-known mathematical methods for correlating a marker combination to a disease status employ methods like discriminant analysis (DA) (e.g., linear-, quadratic-, regularized-DA), Discriminant Functional Analysis (DFA), Kernel Methods (e.g., SVM), Multidimensional Scaling (MDS), Nonparametric Methods (e.g., k-Nearest-Neighbor Classifiers), PLS (Partial Least Squares), Tree-Based Methods (e.g., Logic Regression, CART, Random Forest Methods, Boosting/Bagging Methods), Generalized Linear Models (e.g., Logistic Regression), Principal Components based Methods (e.g., SIMCA), Generalized Additive Models, Fuzzy Logic based Methods, Neural Networks and Genetic Algorithms based Methods. The skilled artisan will have no problem in selecting an appropriate method to evaluate a biomarker combination of the present invention. In one embodiment, the method used in a correlating methylation status of a biomarker combination of the present invention, e.g. to diagnose RB, is selected from DA (e.g., Linear-, Quadratic-, Regularized Discriminant Analysis), DFA, Kernel Methods (e.g., SVM), MDS, Nonparametric Methods (e.g., k-Nearest-Neighbor Classifiers), PLS (Partial Least Squares), Tree-Based Methods (e.g., Logic Regression, CART, Random Forest Methods, Boosting Methods), or Generalized Linear Models (e.g., Logistic Regression), and Principal Components Analysis. Details relating to these statistical methods are found in the following references: Ruczinski et al., 12 J. Of Computational And Graphical Statistics 475-511 (2003); Friedman, J. H., 84 J. Of The American Statistical Association 165-75 (1989); Hastie, Trevor, Tibshirani, Robert, Friedman, Jerome, The Elements of Statistical Learning, Springer Series in Statistics (2001); Breiman, L., Friedman, J. H., Olshen, R. A., Stone, C. J. Classification and regression trees, California: Wadsworth (1984); Breiman, L., 45 Machine Learning 5-32 (2001); Pepe, M. S., The Statistical Evaluation of Medical Tests for Classification and Prediction, Oxford Statistical Science Series, 28 (2003); and Duda, R. O., Hart, P. E., Stork, D. G., Pattern Classification, Wiley Interscience, 2nd Edition (2001). B. Determining Risk of Developing Aggressive/Recurring/Severe RB (Prognosis)

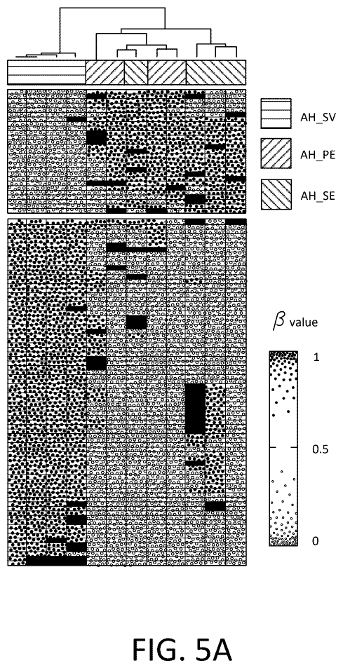

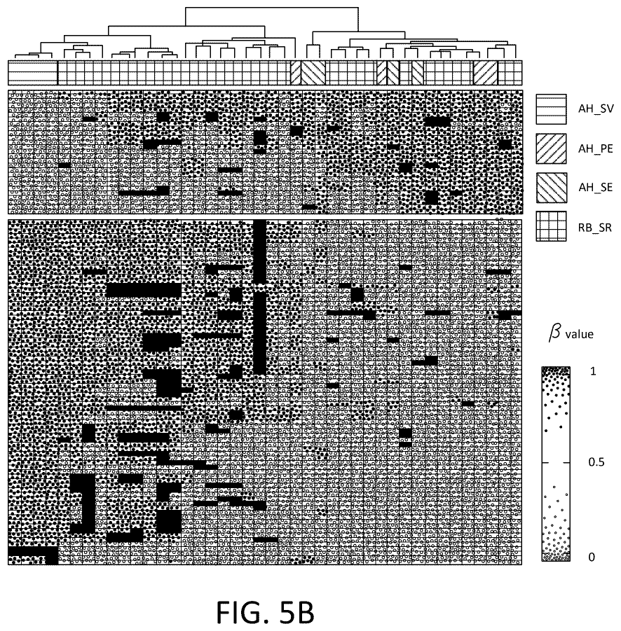

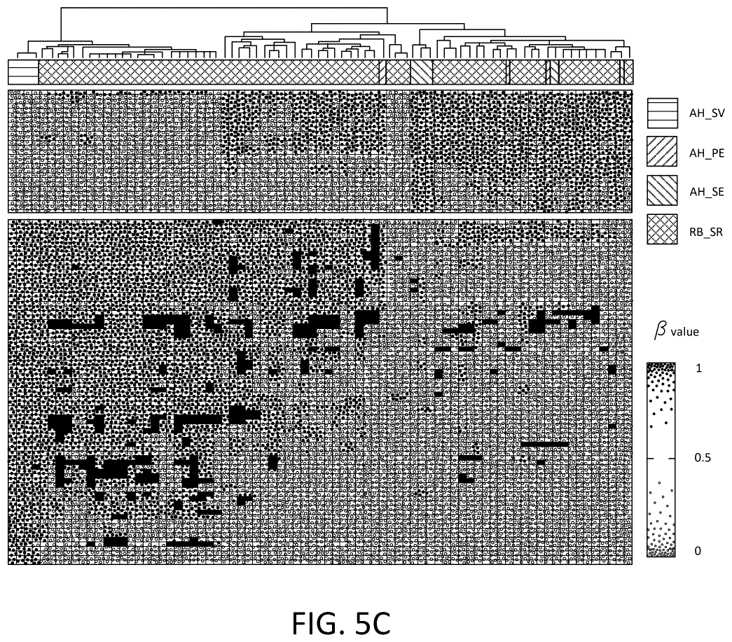

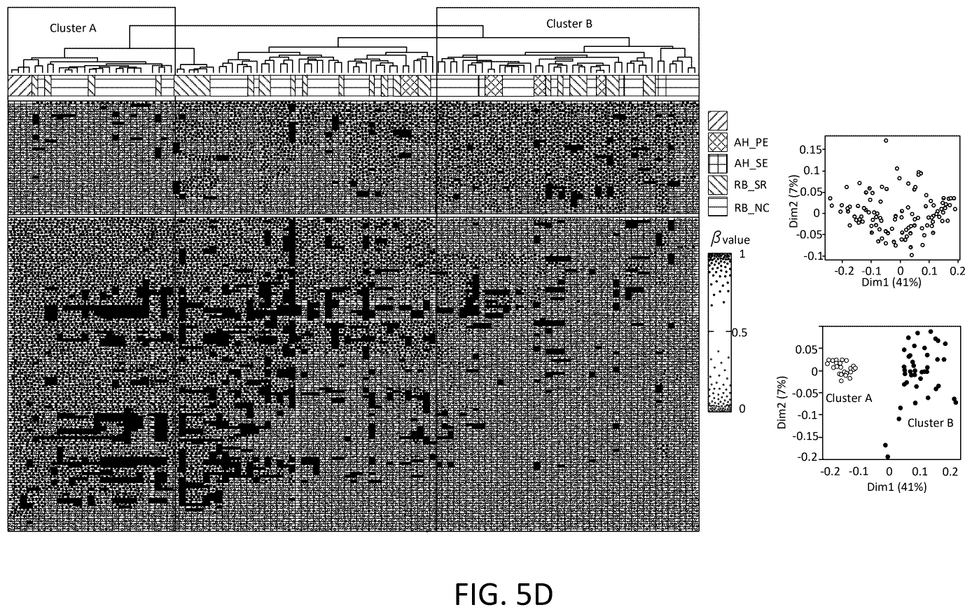

In a specific embodiment, the present invention provides methods for determining the risk of developing aggressive and/or recurring RB in a patient or predicts the need for enucleation of the eye. Biomarker methylation percentages, amounts or patterns are characteristic of various risk states, e.g., high, medium or low. The risk of developing RB is determined by measuring the methylation status of the relevant biomarkers and then either submitting them to a classification algorithm or comparing them with a reference amount, i.e., a predefined level or pattern of methylated (and/or unmethylated) biomarkers that is associated with the particular risk level. See, for example, FIGS. 5, 7 and 8 (status at that locus for Cluster A (good prognosis) vs Cluster B (bad/not good prognosis) is provided).

### C. Determining RB Prognosis

In one embodiment, the present invention provides methods for determining the course of RB in a patient. RB course refers to changes in RB status over time, including RB progression (worsening) and RB regression (improvement). Over time, the amount or relative amount (e.g., the pattern) of hypermethylation and/or hypomethylation of the biomarkers changes. For example, hypermethylation of biomarker “X” and “Y” may be increased with RB. Therefore, the trend of these biomarkers, either increased or decreased methylation over time toward RB or non-RB indicates the course of the disease. Accordingly, this method involves measuring the methylation level or status of one or more biomarkers in a patient at least two different time points, e.g., a first time and a second time, and comparing the change, if any. The course of RB is determined based on these comparisons.

### D. RB Therapies

In certain embodiments of the methods of qualifying RB status, the methods further comprise administering the most appropriate RB therapy. Such therapies include the actions of the physician or clinician subsequent to determining RB status. For example, if a physician makes a diagnosis or prognosis of RB, then a certain regime would follow. An assessment of the course of RB using the methods of the present invention may then require certain RB therapy regimens. In some aspects the biomarker panels can be used to determine the appropriate treatment, and then this information can be used by the physician to prescribe the appropriate type of RB treatment. In some aspects the biomarker panels can be used to determine whether a patient is a candidate for certain treatments. In some aspects the biomarker panels can be used to determine the type of treatment that is most suitable for the patient, such as such drugs/treatments available to physician.

### E. Determining Therapeutic Efficacy

In another embodiment, the present invention provides methods for determining the therapeutic efficacy of a pharmaceutical drug for treating RB. These methods are useful in performing clinical trials of the drug, as well as monitoring the progress of a patient on the drug, or example to determine whether the patient is responding to the drug. Therapy or clinical trials involve administering the drug in a particular regimen. The regimen may involve a single dose of the drug or multiple doses of the drug over time. The doctor or clinical researcher monitors the effect of the drug on the patient or subject over the course of administration. If the drug has a pharmacological impact on the condition, the amounts or relative amounts (e.g., the pattern or profile) of hypermethylation or hypomethylation of one or more of the biomarkers of the present invention may change toward a non-RB profile. Therefore, one can follow the course of the methylation status of one or more biomarkers in the patient during the course of treatment. Accordingly, this method involves measuring methylation levels of one or more biomarkers in a patient receiving drug therapy and correlating the levels with the RB status of the patient (e.g., by comparison to predefined methylation levels of the biomarkers that correspond to different RB statuses). One embodiment of this method involves determining the methylation levels of one or more biomarkers at least two different time points during a course of drug therapy, e.g., a first time and a second time, and comparing the change in methylation levels of the biomarkers, if any. For example, the methylation levels of one or more biomarkers can be measured before and after drug administration or at two different time points during drug administration. The effect of therapy is determined based on these comparisons. If a treatment is effective, then the methylation status of one or more biomarkers will trend toward normal, while if treatment is ineffective, the methylation status of one or more biomarkers will trend toward RB indications. Exemplary therapeutics include for example those listed in FIG. 10.

### F. Kits for the Detection of RB Biomarkers

In another aspect, the present invention provides kits for qualifying RB status, which kits are used to detect or measure the methylation status/levels of the biomarkers described herein. Such kits can comprise at least one polynucleotide that hybridizes to at least one of the diagnostic biomarker sequences of the present invention and at least one reagent for detection of gene methylation. Reagents for detection of methylation include, e.g., sodium bisulfate, polynucleotides designed to hybridize to a sequence that is the product of a biomarker sequence of the invention if the biomarker sequence is not methylated (e.g., containing at least one C→U conversion), and/or a methylation-sensitive or methylation-dependent restriction enzyme. The kits can further provide solid supports in the form of an assay apparatus that is adapted to use in the assay. The kits may further comprise detectable labels, optionally linked to a polynucleotide, e.g., a probe, in the kit. Other materials useful in the performance of the assays can also be included in the kits, including test tubes, transfer pipettes, and the like. The kits can also include written instructions for the use of one or more of these reagents in any of the assays described herein.

In some embodiments, the kits of the invention comprise one or more (e.g., 1, 2, 3, 4, or more) different polynucleotides (e.g., primers and/or probes) capable of specifically amplifying at least a portion of a DNA region of a biomarker of the present invention. Optionally, one or more detectably-labeled polypeptides capable of hybridizing to the amplified portion can also be included in the kit. In some embodiments, the kits comprise sufficient primers to amplify 2, 3, 4, 5, 6, 7, 8, 9, 10, or more different DNA regions or portions thereof, and optionally include detectably labeled polynucleotides capable of hybridizing to each amplified DNA region or portion thereof. The kits further can comprise a methylation-dependent or methylation sensitive restriction enzyme and/or sodium bisulfite.

In some embodiments, the kits comprise sodium bisulfite, primers and adapters (e.g., oligonucleotides that can be ligated or otherwise linked to genomic fragments) for whole genome amplification, and polynucleotides (e.g., detectably labeled polynucleotides) to quantify the presence of the converted methylated and or the converted unmethylated sequence of at least one cytosine from a DNA region of a biomarker of the present invention.

In some embodiments, the kits comprise a microarray comprising, consisting or, or consisting essentially of primers specific for the markers described herein. In more specific embodiments, the kits comprise oligonucleotide sequences which are specific the promoter regions said markers. In more specific embodiments, the kits comprise oligonucleotide sequences which are specific the gene body regions said markers. The sequences of these biomarkers are publicly available.

In some embodiments, the kits comprise methylation sensing restriction enzymes (e.g., a methylation-dependent restriction enzyme and/or a methylation-sensitive restriction enzyme), primers and adapters for whole genome amplification, and polynucleotides to quantify the number of copies of at least a portion of a DNA region of a biomarker of the present invention.

In some embodiments, the kits comprise a methylation binding moiety and one or more polynucleotides to quantify the number of copies of at least a portion of a DNA region of a biomarker of the present invention. A methylation binding moiety refers to a molecule (e.g., a polypeptide) that specifically binds to methylcytosine. Examples include restriction enzymes or fragments thereof that lack DNA cutting activity but retain the ability to bind methylated DNA, antibodies that specifically bind to methylated DNA, etc.).

Without further elaboration, it is believed that one skilled in the art, using the preceding description, can utilize the present invention to the fullest extent. The following example is illustrative only, and not limiting of the remainder of the disclosure in any way whatsoever.

### Example

The following example is put forth so as to provide those of ordinary skill in the art with a complete disclosure and description of how the compounds, compositions, articles, devices, and/or methods described and claimed herein are made and evaluated and are intended to be purely illustrative and are not intended to limit the scope of what the inventors regard as their invention. Efforts have been made to ensure accuracy with respect to numbers (e.g., amounts, temperature, etc.) but some errors and deviations should be accounted for herein. Unless indicated otherwise, parts are parts by weight, temperature is in degrees Celsius or is at ambient temperature, and pressure is at or near atmospheric. There are numerous variations and combinations of reaction conditions, e.g., component concentrations, desired solvents, solvent mixtures, temperatures, pressures and other reaction ranges and conditions that can be used to optimize the product purity and yield obtained from the described process. Only reasonable and routine experimentation will be required to optimize such process conditions.

### Example I—Characterizing DNA Methylation Signatures of Retinoblastoma Using Aqueous Humor Liquid Biopsy

### Introduction

Due to the fundamental role epigenetics plays in this malignancy, a mechanism to assay epigenetic tumor profiles in vivo from eyes undergoing salvage therapy would be highly relevant.

While tumor biopsy is the diagnostic norm for most malignancies, direct tissue biopsy is contraindicated for RB due to the risk of provoking extraocular tumor spread. Until recently this contraindication meant that no molecular tumor information was available unless the eye was enucleated (surgically removed). However, in 2017 it was demonstrated that the aqueous humor (AH), the clear fluid in front of the eye, is an enriched source of tumor-derived cell-free DNA (cfDNA) for RB (10,11), that facilitates analysis of tumor-derived cfDNA in the absence of tumor tissue. Molecular genomic profiling of AH cfDNA opens the door to apply decades of knowledge about RB genomics in an impactful in vivo clinical application. (10,12-14) The addition of epigenetic assays enables better understanding of the role of methylation in orchestrating gene expression in disease initiation and progression. This includes identification of tumors initiated by DNA hypermethylation of the RB1 or other gene promoters that may help stratify patients for epigenetic treatment regimens. Thus, epigenetic analysis of AH cfDNA is a highly desired aspect of an integrated, multimodal liquid biopsy platform.

Provided in this study is genome-scale DNA methylation profiling of paired AH cfDNA and primary RB tumors and integrated the results with existing RB tumor DNA methylation profiles. The methylation profiles of AH cfDNA and primary tumors show high concordance, demonstrating that the AH profiling is a reliable mechanism to evaluate the methylation status of the tumor. Enriched pathway analysis was performed to identify aberrantly methylated genes directly involved in RB tumorigenesis. Finally, the ability to identify RB1 promoter DNA hypermethylation, a known cause of sporadic, non-heritable RB, as well as DNA methylation profiles that may predict an aggressive tumor subtype less likely to respond to medical therapy was demonstrated. The findings support accessible approaches of molecular-based RB diagnosis, and clinical implications of epigenetic dysregulation in RB using this liquid biopsy.

### Methods

### Ethics Approval and Consent to Participate

All human subjects research conducted under this retrospective study was reviewed and approved by the institutional review board at the Children's Hospital Los Angeles (CHLA-17-00248) and following written informed consent from all patients' parents. These experimental methods comply with Helsinki Declaration.

### Consent for Publication

Written information consent for publication was obtained from the parents of patients at enrollment.

### Sample Collection

Tumor and AH specimens were collected from patients with retinoblastoma at Children's Hospital Los Angeles (CHLA). AH collection was performed at diagnosis or at the time of secondary enucleation and at specified clinical intervals throughout therapy; the methods for AH specimen collection and storage has been previously published. (10) No statistical method was used to predetermine sample size. For all participants, treatments were performed per routine CHLA protocol. Treatment regimens were unique for each child and only some children had disease recurrence or enucleation. Therefore, a range of biosamples (0-10 AH samples) were collected for each child depending on clinical course, and blood was drawn alongside AH. HCT116 (CCL-247) human colon cancer cell line was purchased from ATCC with ATCC Cell Line Authentication Service. The growth and passages of cell line was under mycoplasma monitoring.

DNA Extraction from AH, Blood Plasma, Primary Tumor Samples and Cultured Cells

cfDNAs were extracted from AH or blood plasma using the QIAgen QIAamp Circulating Nucleic Acid kit (Qiagen, Valencia, CA USA) as described by the manufacturer. Formalin fixed, paraffin embedded (FFPE) tumor sections were obtained from the CHLA Pathology Laboratory, and FFPE-DNAs were extracted using the QIAgen QIAamp FFPE DN Extraction Mini kit as recommended by the manufacturer. cfDNA and FFPE-DNA concentrations were measured using the Qubit dsDNA High Sensitivity Assay system (ThermoFisher, Waltham, MA USA). For analytical validation purpose, 1 μg genomic DNA from HCT116 (CCL-247) human colon cancer cells in 100 μl ddH₂O was sonicated 200-300 bp fragment sizes that were verified by agarose gel electrophoresis.

### Bisulfite Conversion and Restoration

cfDNA and FFPE-DNA samples were subject to bisulfite conversion using the Zymo EZ DNA methylation kit (Zymo Research, Irvine, CA USA) as specified by the manufacturer. AH cfDNA sample input ranged from 1-2 ng, while FFPE-DNA sample input ranged from 160-240 ng. The amount of bisulfite-converted DNA as well as the completeness of bisulfite conversion for each sample are assessed using a panel of MethyLight-based real-time PCR quality control assays (61). Bisulfite-converted DNAs are then subjected to the Illumina EPIC BeadArrays, as recommended by the manufacturer and described by Moran et al, 2016. (62)

### Targeted Bisulfite Sequencing

Bisulfite-converted DNA was amplified by PCR using the following primers (5′ to 3′) targeting: 1) TFF1 promoter (forward: GGG AAA GAG GGA TTT TTT GAA TT (SEQ ID NO: 1), REVERSE: AAC TAC CAA AAC TAA CTA TAA CCC CAC AA (SEQ ID NO: 2)), 2) HOXC4 promoter forward: ATT TAT TTA AGT GTT AAT TAG GTT GGG T (SEQ ID NO: 3); reverse: AAT TTA AAA TCA TAA CTT ACC AAA ACT CAA (SEQ ID NO: 4)), 3) MNX1 gene body (forward: GGG ATT TGA GGG ATA GTG ATT T (SEQ ID NO: 5), REVERSE: CAA AAT TCA AAT TTC AAC CCC CTA A (SEQ ID NO: 6)) and 4) CELSR3 gene body (forward: AGT ATT GGG AGT TAT TTT TGA GGT T (SEQ ID NO: 7), REVERSE: CAA TCC TCT CCT AAA AAC CAA A (SEQ ID NO: 8)). PCR products were then sequenced using Amplicon-EZ service (Genewiz). Sequencing data were analyzed using the EPIC TABSAT tool. (63)

### EPIC DNA Methylation Data Production

DNA methylation was evaluated using the Illumina Infinium MethylationEPIC (EPIC) BeadArray at the USC Norris Molecular Genomics Core Facility. Specifically, each bisulfite converted sample was whole genome amplified (WGA) and then enzymatically fragmented. Samples were then hybridized overnight to an 8-sample EPIC BeadArray, in which the amplified DNA molecules anneal to locus-specific DNA oligomers linked to individual bead types. The oligomer probe designs follow the Infinium I and II chemistries, in which cy3/cy5-labeled nucleotide base extension follows hybridization to a locus specific oligomer. After the chemistry steps, BeadArrays were scanned using the Illumina iScan system to generate the *.IDAT files in both red and green channels. Raw signal intensities were extracted from the *.IDAT files using the ‘noob’ function in the minfi 1.40.1 R package and the B5 version of the probe manifest. The ‘noob’ function corrects for background fluorescence intensities and red-green dye-bias developed by Triche et al. (21) The beta (β) value represents the DNA methylation score for each data point and is calculated as (M/(M+U)), in which M and U refer to the mean methylated and unmethylated probe signal intensities, respectively. β values range from 0-1, with zero indicating an unmethylated locus and one indicating a fully methylated locus. Measurements in which the fluorescent intensity is not statistically significantly above background signal (detection p value>0.05) as well as non-specific probes and those on the X- and Y-chromosomes were removed from the data set.

### Data Analysis

DNA methylation data of normal retina and RB tumors were obtained from the Gene Expression Omnibus (GEO, GSE57362 and GSE58783). (9,36) Sample purity was assessed using the LUMP (leukocytes unmethylation for purity) assay (64) and 27 samples with LUMP values<0.5 (<50% purity) were removed from further analysis (FIG. S2). Probes related to gender and age, and as well as those overlapping known polymorphisms were also excluded from further analysis. (20,65) Differentially methylated probes were selected using absolute mean β-value difference>0.3 between normal retina and RB tumor samples from GSE58783. Two-sided Welch's t-test (R package matrixTests) was used to identify statistical significance (p value<0.05). The top 10,000 probes with the greatest β-value standard deviation (SD) across the 4 pairs of matched RB primary tumors and AH samples (RB_CHLA_1-4 and AH_CHLA 1-4) were also selected for DNA methylation comparisons. Heatmaps were generated using the R package ComplexHeatmap (66), and multidimensional scaling (MDS) plots were generated using ‘plotMDS’ function in the edgeR package. (67)

### Probe Annotations

Probe annotations were obtained from the B5 version of the Infinium MethylationEPIC probe manifest (hg19, illumina.com). “Promoter” probes were defined as those located at the transcription start site (TSS200 or TSS1500), 5′ untranslated regions (UTR) and the first exon. In addition, “Gene Body” probes were classified as those located within gene bodies and 3′UTRs. The remaining probes were classified as “Other” probes as those not included in Promoter or Gene Body categories.

### Differentially Expressed Genes

Gene expression array data (GSE125903 and GSE111168) (29,30) were used to identify differentially expressed genes between apparently normal retina and RB tumors. The processed expression data were downloaded from BaseSpace correlation engine. (68) The up-regulated genes and down-regulated genes (fold change>2 or <−2) overlapped from both datasets were used for further analysis.

### Pathway and TF Binding Motif Analyses

Pathway analyses for genes regulated by DNA methylation was performed using Ingenuity Pathway Analysis (QIAGEN) (digitalinsights.qiagen.com/products-overview/discovery-insights-portfolio/analysis-and-visualization/qiagen-ipa/). The transcription factor (TF) binding motif prediction was performed using F-match analysis from the TRANSFAC 2.0 database (genexplain, Germany). (32)

### Copy Number Variation Analysis

Copy number variation was detected using the R package SeSAMe. (21,69) The stored normal data (EPIC.5.SigDF.normal) from sesameData was used for normalization.

### Reporting Summary

Further information on research design is available in the Nature Research Reporting Summary linked to this article.

### Data Availability

The raw DNA methylation datasets generated for this study are publicly available at the Gene Expression Omnibus GSE208055 [ncbi.nlm.nih.gov/geo/query/acc.cgi?acc-GSE208055] and GSE211508 [ncbi.nlm.nih.gov/geo/query/acc.cgi?acc-GSE211508]. TRANSFAC (2.0, genexplain, Germany) [genexplain.com/transfac] was used for transcription factor binding sites prediction. The previously published public DNA methylation data of normal retina and RB tumors used in this study are available in GEO database under accession codes GSE57362 [ncbi.nlm.nih.gov/geo/query/acc.cgi?acc-GSE57362] and GSE58783 [ncbi.nlm.nih.gov/geo/query/acc.cgi?acc=GSE58783]. The previously published, processed expression array data (GSE125903 and GSE111168 were downloaded from BaseSpace correlation engine. The remaining data are available within the Article, Supplementary Information, or Source Data File.

### Code Availability

The codes used to generate for the analysis, figures in this study are available at Github repositories (DOI: doi.org/10.5281/zenodo.7005924) 70. R (4.1.1), Rstudio (1.4.1106), and R packages (ggplot2 3.3.5, stats 4.1.1, matrixTests 0.1.9.1, and circlize 0.4.1.4) were used in this study.

### Results

### Validation of DNA Methylation Profiles in RB Specimens

Genome-scale DNA methylation profiling of AH cfDNA and RB tumors was investigated to characterize RB epigenetic changes in vivo. DNA methylation profiles of four paired primary RB tumors and AH cfDNAs (CHLA 1-4) were measured using the Illumina MethylationEPIC (EPIC) DNA methylation BeadArray system. An additional 11 AH cfDNA (CHLA 5-15) samples collected at diagnosis or at the time of enucleation (i.e., surgical removal of the eye) were similarly analyzed. DNA methylation datasets were filtered as per standard to remove data from probes that are: 1) linked to known polymorphisms, 2) located on the X- and Y-chromosomes, and 3) related to aging (FIG. S1). Publicly available DNA methylation data (Illumina Infinium HumanMethylation450, HM450) for primary RB tumors (RB_SR) (n=57) and tumor-adjacent retinas (n=12) as controls (GSE57362) 9 were integrated for validation. By overlapping the EPIC array data with the published HM450 data, a total of 363,579 probes remained for downstream analysis. After filtering for RB tumor purity, 34 primary RB (RB_SR_1-30 and RB_CHLA_1-4) and 15 AH cfDNA (AH_CHLA_1-15) samples were retained for further analysis.

RB tumor-specific DNA methylation changes were identified. Welch's t-test was applied on the filtered 363,579 probe set to identify differentially methylated probes across the 4 primary RB samples from CHLA and the 30 tumors and 12 retinas from a publicly available dataset. With average β value difference>0.3 and p<0.05, 15,483 probes were identified that are significantly differentially methylated between retina and RB samples. DNA methylation changes were identified in 31 of the 34 RB tumors with 3 exceptions (RB SR_18, 21, and 29) that displayed DNA methylation profiles similar to normal retina (FIG. 1A). Approximately 19% of the probes showed strong DNA hypermethylation in RB samples, while 81% displayed DNA hypomethylation, consistent with previous reporting. (8)

Similarly, multidimensional scaling (MDS) of the DNA methylation data revealed that RB tumors mainly clustered separately from normal retina, aside from the three aforementioned tumors (RB_SR_18, 21 and 29) that may represent uninvolved retina (FIG. 1B). The MDS analysis also revealed greater DNA methylation heterogeneity in RB tumors versus the normal retina (FIGS. 1A and 1B).

DNA hyper- or hypomethylation occurs in promoters, gene bodies, enhancer elements, and other as inter-genetic region. However, in the RB-specific group of 15,483 probes, DNA hypermethylated loci were mostly enriched within gene bodies and DNA hypomethylation was most prevalent in gene promoter regions (FIG. 1C). These findings are suggestive of gene expression alterations, as promoter DNA hypomethylation and gene body DNA hypermethylation are correlated with gene activation. (15,16) In addition, DNA methylation in intergenic regions may correlate with chromatin instability and regulation of functional elements, such as enhancers. (17,18) The distribution of RB-specific DNA methylation alterations across various genic regions may provide clues regarding potential gene activity.

DNA methylation profiles in cfDNA of aqueous humor (AH) are reliably assayed and highly concordant to primary RB tumors

The AH cfDNA, like that of other body fluids, is highly fragmented. (19) The Illumina Infinium EPIC DNA Methylation BeadChip is a widely used genome-scale DNA methylation assay (20), however, applying this technology for DNA methylation profiling of highly-degraded, low input DNA samples, such as FFPE-DNA or cfDNA with less than the recommended input DNA amounts (250 ng), presented a challenge.

The lower limits of fragmented DNAs on the EPIC DNA methylation array were evaluated using short DNA fragments. Genomic DNA extracted from the human HCT116 colon cancer cell line was first sonicated to 200-300 bp to match AH cfDNA fragments and then 1 ng, 5 ng, 10 ng, and 20 ng of the fragmentized DNA were subjected to the Illumina Restoration Kit after bisulfite conversion which is recommended for repairing FFPE-DNA samples prior to hybridization to Illumina EPIC DNA methylation arrays. 200 ng DNA was used as a control for bulk DNA amounts commonly evaluated on the EPIC DNA methylation array platform.

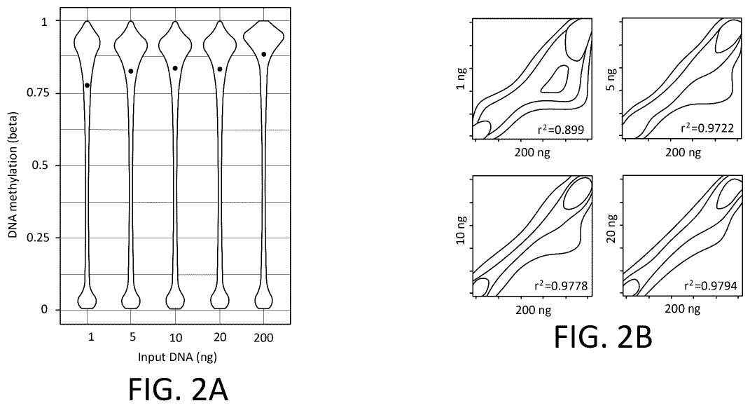

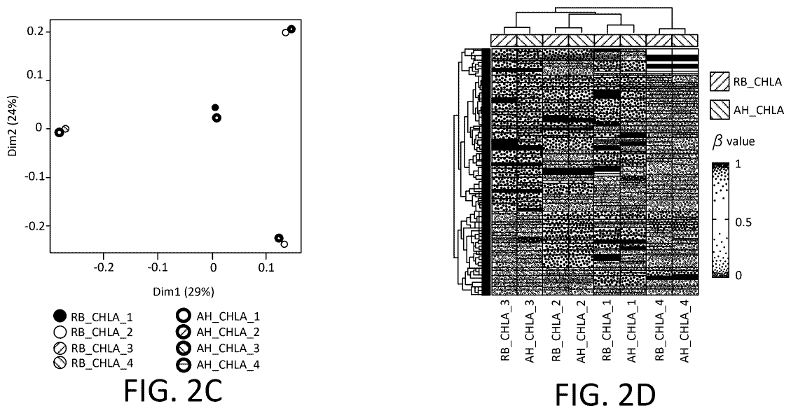

DNA methylation β values for 1 ng, 5 ng, 10 ng and 20 ng of the repaired DNA were plotted versus the 200 ng DNA sample (FIG. 2A). The 1 ng input DNA sample showed some DNA methylation β value skewing compared to the bulk sample, but still showed a high correlation (r2-0.899) to the bulk 200 ng DNA sample (FIG. 2B). The scatterplots and associated correlation coefficients show a strong and reliable association of the 5, 10, 20 ng input DNA samples vs. the bulk 200 ng control sample (r2-0.97-0.98) (FIG. 2B). These findings suggested that Illumina EPIC DNA Methylation assay is applicable for measuring DNA methylation of cfDNA samples with >1 ng input DNA and as such can be applied to the lower amount of cfDNA in the AH.

DNA methylation data on AH cfDNA samples with 1-10 ng input was successfully generated. DNA methylation profiles of four pairs of RB tumors and AHs (CHLA 1-4) demonstrated highly concordant DNA methylation profiles for each tumor-AH pair and distinct separation between different tumor-AH pairs (FIG. 2C). Unsupervised clustering of the most variably methylated probes across all four tumor-AH pairs also highlighted differential DNA methylation among these four patients and highly concordant DNA methylation profiles between each RB tumor and its corresponding paired AH (FIG. 2D); this demonstrates that the AH could be used in the absence of tumor (e.g., from eyes that have not been surgically removed) to accurately assay the methylation signature of the tumor in vivo. Copy number analysis of the EPIC DNA methylation data set18,21 also showed high concordance between each primary RB tumor and its paired AH cfDNA specimen (FIG. S3A).

An additional set of 11 AH samples from CHLA were included for further comparison, such that a total of n=15 AH cfDNA specimens were analyzed. All 15 AH_CHLA samples showed the RB-specific DNA methylation pattern by unsupervised clustering (FIG. 3A) and MDS (FIG. 3B) analyses. The RB-specific DNA methylation profiles were not detected in cfDNA isolated from blood plasma in two RB patients but rather clustered with white blood cell DNA isolated from RB patients (FIG. S4) as expected since the disease was confined to the eye without high tumor fraction in the blood. (12,22)

Analyses of gene-level DNA methylation in FIG. 3A revealed RB1 promoter DNA hypermethylation in five samples (RB_CHLA_3, AH_CHLA_3, RB_SR_16, RB_SR_24, and RB_SR_30) consistent with RB1 inactivation via epigenetic silencing; a known mechanism of non-heritable RB1 inactivation. Previously this could only be determined with access to tumor tissue, however identical RB1 promoter DNA hypermethylation was detected in cfDNA of AH and from tumor DNA from the same enucleated eye (RB_CHLA_3 and AH_CHLA_3) further demonstrating that RB1 promoter DNA hypermethylation can be reliably detected via ocular AH liquid biopsy in the absence of tumor tissue (FIG. 3C).

Overexpression and/or amplification of MYCN and SYK have been demonstrated to highly correlated to RB tumorigenesis and considered as potential therapeutic targets. (23-25) In the cohort, the majority of RB tumors demonstrated MYCN and SYK promoter DNA hypomethylation (associated with gene activation); this profile was similarly identified in all AH samples as compared to apparently normal retinal tissues (FIG. 3C), suggesting that these targets can be detected via AH methylation profiling.

Characterization of Genes with RB-Associated DNA Methylation Profiles and their Involvement in RB Tumorigenesis

While most cancer-specific DNA methylation alterations do not result in altered gene expression (16, 26, 27), promoter DNA methylation is negatively correlated with gene expression and gene body DNA methylation is positively associated with gene expression. (15,28) To characterize the extent to which promoter and gene body DNA methylation affect gene expression, publicly-available RNA sequencing (RNA200 seq) data (GSE125903 and GSE111168) (29,30) were used due to limited availability of primary RB samples.

First, DNA methylation probes exhibiting RB-specific DNA methylation changes (delta β value>0.3) in promoter or gene body regions were identified by comparing normal retina and primary RB samples (FIG. 1). In total, promoter DNA hypermethylation (978 probes), promoter DNA hypomethylation (4,949 probes), gene body DNA hypermethylation (1,178 probes) and gene body DNA hypomethylation (3,856 probes) were identified (FIG. 4A). Additionally, upregulation of 889 and downregulation of 382 genes in RB were uncovered. After integrating the RB-specific promoter and gene body DNA methylation (FIG. 4A) with differential gene expression data, 294 genes were identified that show potential gene regulation by aberrant DNA methylation directly in RB (FIG. 4B). These genes include those upregulated and correlated with promoter DNA hypomethylation (n=172) or gene body DNA hypermethylation (n=37), as well as those down-regulated and correlated with promoter DNA hypermethylation (n=67) or gene body DNA hypomethylation (n=18) (FIG. 4B). Although Illumina-based DNA methylation data are reliable and have been validated using pyrosequencing and targeted bisulfite sequencing (27,31), the EPIC DNA methylation array data was confirmed by performing targeted bisulfite sequencing of four gene regions (TFF1 and HOXC4 promoters and MNX1 and CELSR3 gene bodies) on 10 additional primary RB tumors and one apparently healthy retina (FIG. S5). The EPIC array and targeted bisulfite sequencing DNA methylation data are highly consistent at these four loci.

The potential roles of epigenetic-directed gene expression during RB tumorigenesis remain unclear. Core analysis in Ingenuity Pathway Analysis (IPA, Qiagen) was performed on the set of 294 differentially expressed genes to understand their potential functional profiles for RB tumorigenesis (FIGS. 4B and 4C). The Graphical Summary algorithm predicted downregulation of tumor suppressor pathways involving p53, RB1, CDKN2A/p16, and CDKN1A/p21, and activation of oncogenic pathways involving E2F1, E2F2, E2F3, and MYC (FIG. 4C). Furthermore, TRANSFAC analysis was used to identify transcription factors (TFs) involved in the regulation of these pathways or genes. (32) The top over-represented transcription factors binding sites included oncogenic regulators involved in ER (Estrogen receptor), Ras (RREB-1, Ras responsive element binding protein 1), E2F, MYC (MAZ, MYC associated zinc finger protein), NF-kB, and EGR1 (Early growth response protein 1) signaling pathways (FIG. S6).

These pathways are known to be involved in RB tumorigenesis and contribute to upregulation of Aryl Hydrocarbon Receptor (AhR) signaling, Estrogen-mediated S-phase entry for cancer cell proliferation, and others.33-35 These findings suggest that in addition to genetic alterations such as pathogenic RB1 variants, DNA methylation-regulated genes related to cancer aggressiveness can be involved in downregulation of tumor suppressor pathways as well as upregulation of oncogenic pathways that contribute to RB tumorigenesis. Further detailed analysis of Estrogen-mediated S-phase pathway elements revealed several key downstream signaling genes that are upregulated in association with promoter DNA hypomethylation in RB tumors, including CCNA1 and CCNA2 for Cyclin A, CCNE1 and CCNE2 for Cyclin E, as well as E2F1, E2F2 and CDC2 (FIG. 4D). Interestingly, the data also showed that these genes, especially in downstream of RB1 such as Cyclin A, Cyclin E, and CDC2, can be upregulated by promoter DNA hypomethylation independent of germline RB1 mutation status (FIG. 4E).

AH cfDNA Methylation Profiles are Associated with RB Tumor Aggressiveness

To explore the potential of epigenomic prognostic biomarkers to predict eye salvage via AH, EPIC DNA methylation data was analyzed from 12 AHs from eyes with different clinical outcomes, including four eyes salvaged with therapy (SV) with at least Ing AH cfDNA (AH_CHLA_6 removed), four primarily enucleated eyes (PE) without initial medical intervention and four secondary enucleations (SE) in which the eye failed attempted treatment. Three AH samples with low data quality were excluded. Specifically, AH cfDNA methylation profiles between salvage (AH_CHLA_5, 8, 9, and 10), primary enucleation (AH_CHLA_1, 2, 3, and 4) and secondary enucleation (AH_CHLA_11, 12, 13, and 14) cases were analyzed using heatmap representation after unsupervised clustering. In total, 1,092 probes were identified that are significantly differentially methylated (Δβ>0.4, p<0.01) between the salvage (n=4) and enucleation groups (n=4 PE and n=4 SE) (FIG. 5A). As expected, based on previous work (12) and that of Liu et al. (36), salvaged eyes had fewer copy number alterations than enucleated eyes, especially for gain of 1q, 6p and loss of 16q in current dataset (FIG. S3B).

To determine if these same probes could be used to distinguish primary tumors, DNA methylation data for this set of 1,092 differentially methylated probes was further applied to the above-described 30 primary RB tumors (RB_SR, GSE57362). Interestingly, in the larger data set there remained differential methylation between the salvaged group and the enucleated tumors (FIG. 5B). For further validation, this comparison was repeated with a second DNA methylation data set of 67 primary RB tumor samples (RB_NC, GSE58783) (36) using unsupervised clustering (FIG. 5C). The distinguishable pattern (FIG. 5A) between salvaged samples and enucleation samples in DNA from primary tumors or AH liquid biopsy (FIGS. 5B and 5C) suggests that DNA methylation analyses of AH cfDNA samples from RB eyes may be used to predict eye salvage.

To further investigate if the AH cfDNA methylation signature discriminates distinct tumor subgroups, unsupervised clustering of the merged datasets were performed: 4 AH cfDNAs collected at diagnosis from eyes that were salvaged, 8 AH cfDNAs from eyes that were eventually enucleated, and 97 (30+67) enucleated samples from primary RB tumors (FIG. 5D). While the heatmap representation after unsupervised clustering (a) and MDS analyses (b) demonstrate the variability of methylation signatures in RB tumors, there were two unique patterns on the edges of the spectrum. A subset of tumors that had a similar methylation signature to CHLA salvaged tumors (Cluster A) and an opposite signature more typical of the tumors enucleated at CHLA were identified, either primarily or secondarily (Cluster B). The distribution of salvaged tumors on one arm and subsequently enucleated tumors on the other arm was significant (p<0.01). 320 significantly differentially methylated probes (A B>0.4, p<0.01) spanning 185 unique genes between the Cluster A and Cluster B that were well separated using MDS analysis in (c) of FIG. 5D were further identified.

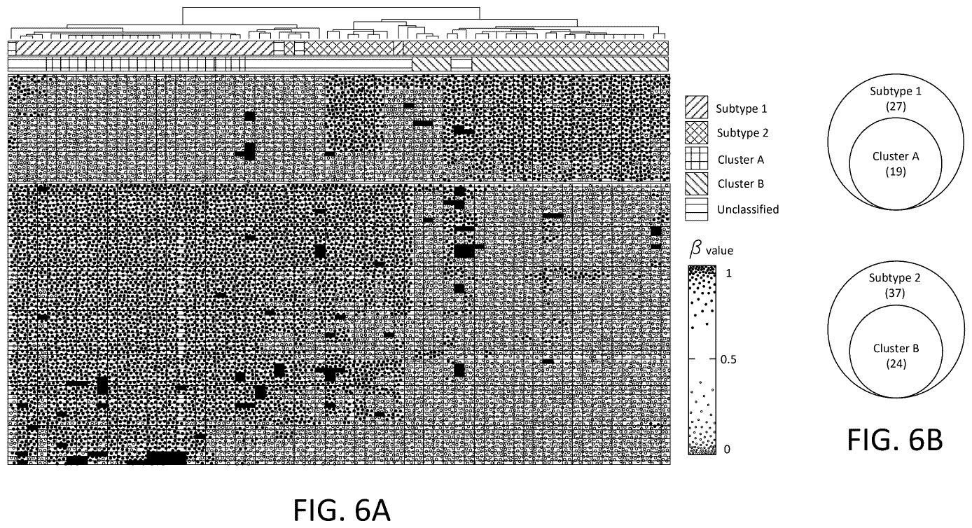

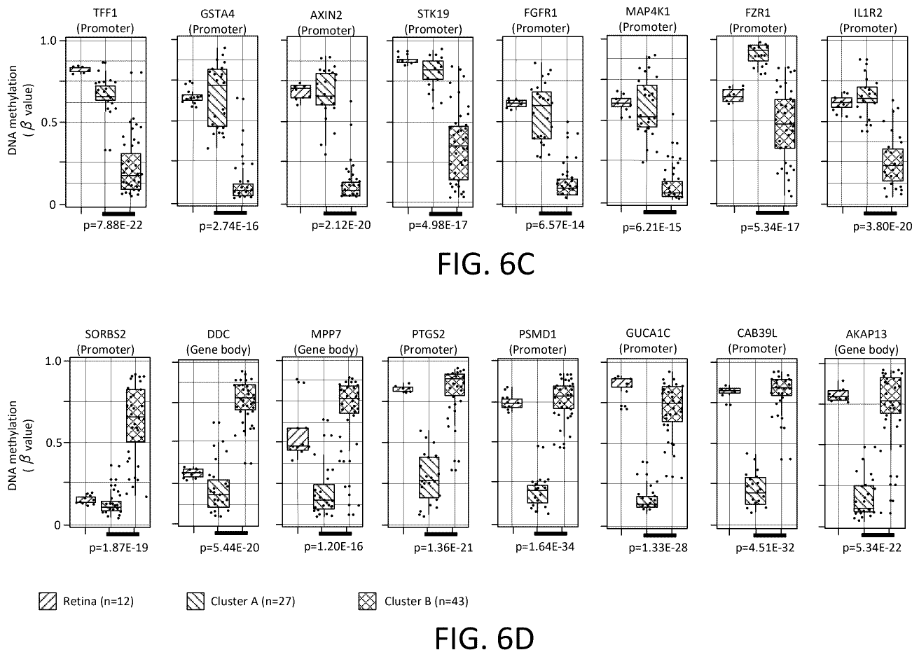

To better understand the characteristics of Cluster A and Cluster B tumors, the data (GSE58783) from Liu et al. (36) was investigated. Liu et al. identified two RB tumor subtypes (Subtype 1 and Subtype 2) based on DNA methylation, copy number variation and gene expression profiles from 67 enucleated RB samples with DNA methylation data. Subtype 1 RB tumors maintained a differentiated state, while Subtype 2 RB tumors displayed more aggressive disease that is associated with dedifferentiation, stemness features and expression of neuronal markers. (36) The Subtype assignments were applied with Cluster A and B subgrouping, which were distinguished by 320 differentially methylated probes in AH cfDNA (FIGS. 6A and 6B) and compared the instant assignments with theirs. Cluster A tumors (n=19) fully overlapped with differentiated Subtype 1 (n=27) cases while Cluster B tumors (n=24) also fully overlapped with de-differentiated Subtype 2 (n=37) cases as described by unsupervised clustering and Venn diagram (FIGS. 6A and 6B), demonstrating the classifier for treatment outcome prediction disclosed herein is consistent with their classifier for disease aggressiveness. Taken together, these data suggested that a more aggressive RB tumor subtype could be predicted by cfDNA methylation profiles of AH liquid biopsies.

A literature search was performed to identify known oncogenesis genes that display the greatest extent of differential DNA methylation from the identified 185 genes based on 320 differentially methylated probes between Clusters A and B. The examples of those genes that had either DNA hypomethylation in Cluster B tumors as compared to Cluster A tumors (FIG. 6C), or DNA hypermethylation in Cluster B tumors as compared to Cluster A tumors are shown in FIG. 6D. Unexpectedly, by including normal retina DNA methylation as a control (bright green), it was determined that the majority (7/8) of the hypermethylated genes in Cluster A are similarly hypermethylated in normal retina, while these genes remain unmethylated in Cluster B tumors (FIG. 6C). However, the FZR1 promoter displayed similar and moderate DNA methylation levels in normal retina and Cluster B tumors, but DNA hypermethylation in Cluster A tumors. Concurrently, most (5/8) hypermethylated genes in Cluster B were similar to normal retina, while these genes are unmethylated in Cluster A tumors thus it is the hypomethylation that is aberrant (compared to retina) (FIG. 6D). However, it should be noted that SORBS2 promoter and DDC and MPP7 gene bodies showed little to moderate DNA methylation in normal retina and Cluster A tumors, but DNA hypermethylation in Cluster B tumors (FIG. 6D).

Most of the genes identified are known to be involved in tumor aggressiveness (FIGS. 6C and D) and may directly contribute to an RB phenotype that is more likely to fail treatment (or require more aggressive intervention to salvage the eye). TFF1 overexpression is associated with aggressive disease and correlated with dedifferentiation with stemness features and higher risk of metastasis in RB36,37, while GSTA4 overexpression plays a key role for resistance of cisplatin-chemotherapy. (38-40) AXIN2 expression is driven by MYC and overexpressed in multiple human cancers critical to maintain cancer cell aggressiveness via regulation of the beta catenin/wnt pathway. (41) STK19 is an NRAS-activating kinase and the over-expression of which leads to cancer invasion and is a potential therapeutic target.42 FRGR1 is involved in cancer cell proliferation and metastasis (43), and ILIR2 promotes cancer cell proliferation and invasion and ILIR2 blockade suppresses tumor progression44 (FIG. 6C).

FZR1 has been described as both a tumor suppressor and oncoprotein. FZR1 promoter DNA hypermethylation in Cluster B tumors may correlate with FZR1 loss that results in increased sensitivity to DNA damage and resistance to chemotherapy. (45) In addition, SORBS2 and CAB39L have been suggested as potential tumor suppressors (46,47) and silencing of these genes by promoter DNA hypermethylation in Cluster B RBs may contribute to tumor aggressiveness (FIG. 6D). Taken together, these genes not only serve as prognostic biomarkers to predict eye salvage, tumor aggressiveness and likely response to treatment, but opens the door to future applications of predictive medicine by facilitating an in vivo evaluation of potential therapeutic targets for patients with RB, particularly those with more aggressive disease (FIG. 6).

### DISCUSSION

There exists a significant body of research into the genetic, genomic and epigenomic alterations of RB. However, this research was done on tumor tissue from surgically removed (enucleated) eyes. Due to the discernable risk of tumor dissemination after tumor biopsy (48,49), obtaining RB tissue DNA has been challenging aside from enucleated specimens. Thus, any application of molecular diagnostic or prognostic biomarkers, or use of these biomarkers for personalized medicine, was limited by the lack of tumor tissue at diagnosis or during therapy. Thus, utilization of a liquid biopsy approach may address this concern for RB and other malignancies in which tumor biopsy is not readily accessible.

Research into the AH liquid biopsy has demonstrated that this biofluid is an enriched source of tumor-derived DNA. In previous work, a prognostic genomic biomarker in the AH cfDNA was identified, gain of chrop, which could predict eye salvage better than currently used clinical classification schemes. (10, 13, 14) However, this molecular prognostication analysis relies on the presence of somatic copy number alterations in Rb genomes, which not all tumors harbor. Additionally, based on previous investigations (6), approximately 13% of Rb tumors are initiated by RB1 promoter hypermethylation. The other genes involved in aggressive RB tumorigenesis, and more importantly, whether they differ between more and less aggressive RB phenotypes, remains an area of active investigation. (36)

Aberrant DNA methylation is a common event in most malignancies and a reliable tumor marker for diagnosis and prognosis, however most of the defined alterations appear to be passenger events that do not actually lead to gene expression changes. (27,50-52) The ability to identify RB-derived molecular aberrations in cfDNA isolated from the aqueous humor provides an opportunity to characterize genetic and epigenetic features of eye tumors in vivo while RB patients are actively undergoing therapy. (12-14)

In this study, the DNA methylation profiles of primary RB specimens, cfDNA from AH of RB patients, and normal retina tissues which are available in public databanks, were compared. The analyses revealed that DNA methylation profiles from the tumor-derived cfDNA in the AH is representative of RB tumor tissue (9), thus demonstrating the AH is a reliable biofluid for methylation profiling of the tumor. In this subset, a patient with hypermethylation of the promotor, a known mechanism of tumorigenesis, was demonstrated. Previously this could only have been identified from tumor tissue in enucleated eyes; however, the work herein demonstrates the ability to detect this from the aqueous humor (alongside methylation signatures of multiple other genes including SYK, MYCN and others). This opens a multi-omics approach to AH analysis, enabling us to characterize the global methylation pattern of RB tumors in vivo at diagnosis and during therapy, thus obviating the need for tumor tissue. Moreover, unlike genetic alterations, RB1 epigenetic silencing is reversible and may be a therapeutic target of DNA methylation inhibitors. These have not been used for the treatment of RB but have been used for treatment of several cancer types in clinical trials. (28,53)

By integrating DNA methylation and gene expression data from primary RB tumors, 294 genes were identified that are directly regulated by promoter or gene body DNA methylation. The established correlation between DNA methylation and gene expression in these genes suggests that these DNA methylation markers can be used in place of RNA- or protein-based gene expression profiling. For example, the key therapeutic target genes of RB1, SYK, MYCN, E2Fs expression status can be predicted by their DNA methylation status. As expected, RB1 was also identified as the potential top upstream regulator of genes involved in Estrogen-mediated S-phase entry, and downregulation of downstream of these genes mimic decreased RB1 activity. (54,55) Notably, the expression changes of these downstream genes controlled by DNA methylation may directly alter the Estrogen-mediated S-phase entry independent of RB1 mutation status, suggesting that DNA methylation is an independent driving force for RB tumorigenesis. Interestingly, these genes also are directly regulated by oncogenic regulators, such as ER, Ras, E2F, MYC, NF-kB and EGR1 signaling.

Potential prognostic methylation markers for tumor aggressiveness were identified from RB eyes in vivo via an AH liquid biopsy taken at diagnosis or during active treatment. This liquid biopsy approach enabled assay of tumor-derived cfDNA in the absence of tumor tissue. Using the AH, a clear differential methylation signature was identified between eyes that were salvaged with therapy (Cluster A) and eyes that failed therapy and were enucleated (Cluster B). This AH methylation signature is highly concordant with previous genomic and epigenetic analyses of RB tumors. (36) This work builds upon the work from Liu et al. (36), suggesting molecular subtypes of RB. This work allows for detection of these subtypes from the AH in vivo. This can directly impact these young patients with RB by allowing the clinician to understand the state of the tumor, and combined with clinical features, the likelihood of salvage with various therapies.

TFF1, GSTA4, AXIN2, ILIR2, STK19 and FRGR1 Promoter DNA hypomethylation in aggressive RBs (Cluster B) identified in this study may result in gene overexpression, thereby leading to tumor dedifferentiation with stemness features (36), resistance to cisplatin chemotherapy (39,40), maintained cancer cell aggressiveness by protecting the tumor from oxidation stress and ensuring MYC-driven transcription (41,56), cancer invasion (42,43), and T421 cell suppression. (44,57) Furthermore, silencing of FZR1 due to promoter DNA hypermethylation in Cluster B cases may decrease sensitivity to chemotherapy (45) and suppress antitumor immunity. (44,57) Interestingly, promoter DNA methylation of tumor suppressor genes SORBS2 and CAB39L may also contribute to tumor aggressiveness characteristic of Cluster B cases. (46,47)

DNA methylation is a stable epigenetic modification that is routinely assayed by several technologies. (58) Isolating cfDNA from AH is now a well-established procedure (12,59), and therefore can easily be applied to RB patients in the clinical setting. Characterizing RB-specific DNA methylation markers in AH cfDNA provides a foundation for future applications in the clinical diagnosis and prognostication of RB and as well as potential for precision medicine-based treatment approaches.

### BIBLIOGRAPHY

1 Rodriguez-Galindo, C., Orbach, D. B. & VanderVeen, D. Retinoblastoma. Pediatr Clin North Am 62, 201-223 (2015). https://doi.org:10.1016/j.pcl.2014.09.014
2 Kaewkhaw, R. & Rojanaporn, D. Retinoblastoma: Etiology, Modeling, and Treatment. Cancers (Basel) 12 (2020). https://doi.org:10.3390/cancers12082304
3 Knudson, A. G., Jr. Mutation and cancer: statistical study of retinoblastoma. Proc Natl Acad Sci USA 68, 820-823 (1971). https://doi.org:10.1073/pnas.68.4.820
4 Sakai, T. et al. Allele-specific hypermethylation of the retinoblastoma tumor581 suppressor gene. Am J Hum Genet 48, 880-888 (1991).
5 Stirzaker, C. et al. Extensive DNA methylation spanning the Rb promoter in retinoblastoma tumors. Cancer Res 57, 2229-2237 (1997).
6 Greger, V. et al. Frequency and parental origin of hypermethylated RB1 alleles in retinoblastoma. Hum Genet 94, 491-496 (1994). https://doi.org:10.1007/BF00211013
7 Benavente, C. A. & Dyer, M. A. Genetics and epigenetics of human retinoblastoma. Annu Rev Pathol 10, 547-562 (2015). https://doi.org:10.1146/annurev-pathol-012414-040259
8 Aldiri, I. et al. The Dynamic Epigenetic Landscape of the Retina During Development, Reprogramming, and Tumorigenesis. Neuron 94, 550-568 e510 (2017). https://doi.org:10.1016/j.neuron.2017.04.022
9 Berdasco, M. et al. DNA Methylomes Reveal Biological Networks Involved in Human Eye Development, Functions and Associated Disorders. Sci Rep 7, 11762 (2017). https://doi.org:10.1038/s41598-017-12084-1
10 Berry, J. L. et al. Potential of Aqueous Humor as a Surrogate Tumor Biopsy for Retinoblastoma. JAMA Ophthalmol 135, 1221-1230 (2017). https://doi.org:10.1001/jamaophthalmol.2017.4097
11 Kitazawa, K. et al. Safety of anterior chamber paracentesis using a 30-gauge needle integrated with a specially designed disposable pipette. Br J Ophthalmol 101, 548-550 (2017). https://doi.org:10.1136/bjophthalmol-2016-309650
12 Gerrish, A., Jenkinson, H. & Cole, T. The Impact of Cell-Free DNA Analysis on the Management of Retinoblastoma. Cancers (Basel) 13 (2021). https://doi.org:10.3390/cancers13071570
13 Xu, L. et al. Establishing the Clinical Utility of ctDNA Analysis for Diagnosis, Prognosis, and Treatment Monitoring of Retinoblastoma: The Aqueous Humor Liquid Biopsy. Cancers (Basel) 13 (2021). https://doi.org:10.3390/cancers13061282
14 Xu, L. et al. Simultaneous identification of clinically relevant RB1 mutations and copy number alterations in aqueous humor of retinoblastoma eyes. Ophthalmic Genet 41, 526-532 (2020). https://doi.org:10.1080/13816810.2020.1799417
15 Yang, X. et al. Gene body methylation can alter gene expression and is a therapeutic target in cancer. Cancer Cell 26, 577-590 (2014). https://doi.org:10.1016/j.ccr.2014.07.028
16 Liu, M. et al. Integrative Epigenetic Analysis Reveals Therapeutic Targets to the DNA Methyltransferase Inhibitor Guadecitabine (SGI-110) in Hepatocellular Carcinoma. Hepatology 68, 1412-1428 (2018). https://doi.org:10.1002/hep.30091
17 Berman, B. P. et al. Regions of focal DNA hypermethylation and long-range hypomethylation in colorectal cancer coincide with nuclear lamina-associated domains. Nat Genet 44, 40-46 (2011). https://doi.org:10.1038/ng.969
18 Zhou, W. et al. DNA methylation loss in late-replicating domains is linked to mitotic cell division. Nat Genet 50, 591-602 (2018). https://doi.org:10.1038/s41588-018-0073-4
19 Diaz, L. A., Jr. & Bardelli, A. Liquid biopsies: genotyping circulating tumor DNA. J Clin Oncol 32, 579-586 (2014). https://doi.org:10.1200/JCO.2012.45.2011
20 Zhou, W., Laird, P. W. & Shen, H. Comprehensive characterization, annotation and innovative use of Infinium DNA methylation BeadChip probes. Nucleic Acids Res 45, e22 (2017). https://doi.org:10.1093/nar/gkw967
21 Triche, T. J., Jr., Weisenberger, D. J., Van Den Berg, D., Laird, P. W. & Siegmund, K. D. Low-level processing of Illumina Infinium DNA Methylation BeadArrays. Nucleic Acids Res 41, e90 (2013). https://doi.org/10.1093/nar/gkt090
22 Polski, A. et al. Cell-Free DNA Tumor Fraction in the Aqueous Humor Is Associated With Therapeutic Response in Retinoblastoma Patients. Transl Vis Sci Technol 9, 30 (2020). https://doi.org:10.1167/tvst.9.10.30
23 Zhang, J. et al. A novel retinoblastoma therapy from genomic and epigenetic analyses. Nature 481, 329-334 (2012). https://doi.org:10.1038/nature10733
24 Wu, N. et al. A mouse model of MYCN-driven retinoblastoma reveals MYCN638 independent tumor reemergence. J Clin Invest 127, 888-898 (2017). https://doi.org:10.1172/JCI88508
25 Sradhanjali, S. et al. The Oncogene MYCN Modulates Glycolytic and Invasive Genes to Enhance Cell Viability and Migration in Human Retinoblastoma. Cancers (Basel) 13 (2021). https://doi.org:10.3390/cancers13205248
26 Pandiyan, K. et al. Functional DNA demethylation is accompanied by chromatin accessibility. Nucleic Acids Res 41, 3973-3985 (2013). https://doi.org:10.1093/nar/gkt077
27 Becket, E. et al. Identification of DNA Methylation-Independent Epigenetic Events Underlying Clear Cell Renal Cell Carcinoma. Cancer Res 76, 1954-1964 (2016). 648 https://doi.org:10.1158/0008-5472.CAN-15-2622
28 Liang, G. & Weisenberger, D. J. DNA methylation aberrancies as a guide for surveillance and treatment of human cancers. Epigenetics 12, 416-432 (2017). https://doi.org:10.1080/15592294.2017.1311434
29 Rajasekaran, S. et al. Non-coding and Coding Transcriptional Profiles Are Significantly Altered in Pediatric Retinoblastoma Tumors. Front Oncol 9, 221 (2019). https://doi.org:10.3389/fonc.2019.00221
30 Chai, P. et al. Dynamic chromosomal tuning of a novel GAU1 Incing driver at chr12p13.32 accelerates tumorigenesis. Nucleic Acids Res 46, 6041-6056 (2018). https://doi.org:10.1093/nar/gky366
31 Su, S. F. et al. A panel of three markers hyper- and hypomethylated in urine sediments accurately predicts bladder cancer recurrence. Clin Cancer Res 20, 1978-1989 (2014). https://doi.org:10.1158/1078-0432.CCR-13-2637
32 Matys, V. et al. TRANSFAC and its module TRANSCompel: transcriptional gene regulation in eukaryotes. Nucleic Acids Res 34, D108-110 (2006). https://doi.org:10.1093/nar/gkj 143
33 Murray, I. A., Patterson, A. D. & Perdew, G. H. Aryl hydrocarbon receptor ligands in cancer: friend and foe. Nat Rev Cancer 14, 801-814 (2014). https://doi.org:10.1038/nrc3846
34 Foster, J. S., Henley, D. C., Bukovsky, A., Seth, P. & Wimalasena, J. Multifaceted regulation of cell cycle progression by estrogen: regulation of Cdk inhibitors and Cdc25A independent of cyclin D1-Cdk4 function. Mol Cell Biol 21, 794-810 (2001). https://doi.org:10.1128/MCB.21.3.794-810.2001
35 Doisneau-Sixou, S. F. et al. Estrogen and antiestrogen regulation of cell cycle progression in breast cancer cells. Endocr Relat Cancer 10, 179-186 (2003). https://doi.org:10.1677/erc.0.0100179
36 Liu, J. et al. A high-risk retinoblastoma subtype with stemness features, dedifferentiated cone states and neuronal/ganglion cell gene expression. Nat Commun 12, 5578 (2021). https://doi.org:10.1038/s41467-021-25792-0
37 Busch, M. A. et al. TFF1 in Aqueous Humor-A Potential New Biomarker for Retinoblastoma. Cancers (Basel) 14 (2022). https://doi.org:10.3390/cancers14030677
38 Zhu, H. et al. Molecular mechanisms of cisplatin resistance in cervical cancer. Drug Des Devel Ther 10, 1885-1895 (2016). https://doi.org:10.2147/DDDT.S106412
39 Park, H. J. et al. GSTA4 mediates reduction of cisplatin ototoxicity in female mice. Nat Commun 10, 4150 (2019). https://doi.org:10.1038/s41467-019-12073-0
40 Kalinina, E. V. et al. Expression of genes of glutathione transferase isoforms GSTP1-1, GSTA4-4, and GSTK1-1 in tumor cells during the formation of drug resistance to cisplatin. Bull Exp Biol Med 154, 64-67 (2012). https://doi.org:10.1007/s10517-012-1876-4
41 Li, Y. et al. c-Myb Enhances Breast Cancer Invasion and Metastasis through the Wnt/beta-Catenin/Axin2 Pathway. Cancer Res 76, 3364-3375 (2016). https://doi.org:10.1158/0008-5472.CAN-15-2302
42 Yin, C. et al. Pharmacological Targeting of STK19 Inhibits Oncogenic NRAS-Driven Melanomagenesis. Cell 176, 1113-1127 e1116 (2019). https://doi.org:10.1016/j.cell.2019.01.002
43 Wang, K. et al. FGFR1-ERK1/2-SOX2 axis promotes cell proliferation, epithelial695 mesenchymal transition, and metastasis in FGFR1-amplified lung cancer. Oncogene 37, 5340-5354 (2018). https://doi.org:10.1038/s41388-018-0311-3
44 Zhang, L. et al. IL1R2 Blockade Suppresses Breast Tumorigenesis and Progression by Impairing USP15-Dependent BMI1 Stability. Adv Sci (Weinh) 7, 1901728 (2020). https://doi.org:10.1002/advs.201901728
45 Ishizawa, J. et al. FZR1 loss increases sensitivity to DNA damage and consequently promotes murine and human B-cell acute leukemia. Blood 129, 1958-1968 (2017). https://doi.org:10.1182/blood-2016-07-726216
46 Li, W. et al. CAB39L elicited an anti-Warburg effect via a LKB1-AMPK-PGC1alpha axis to inhibit gastric tumorigenesis. Oncogene 37, 6383-6398 (2018). https://doi.org:10.1038/s41388-018-0402-1
47 Zhao, L. et al. The RNA binding protein SORBS2 suppresses metastatic colonization of ovarian cancer by stabilizing tumor-suppressive immunomodulatory transcripts. Genome Biol 19, 35 (2018). https://doi.org:10.1186/s13059-018-1412-6
48 Karcioglu, Z. A. Fine needle aspiration biopsy (FNAB) for retinoblastoma. Retina 22, 707-710 (2002). https://doi.org:10.1097/00006982-200212000-00004
49 Munier, F. L. et al. Conservative management of retinoblastoma: Challenging orthodoxy without compromising the state of metastatic grace. “Alive, with good vision and no comorbidity”. Prog Retin Eye Res 73, 100764 (2019). https://doi.org:10.1016/j.preteyeres.2019.05.005
50 Jones, P. A. & Baylin, S. B. The fundamental role of epigenetic events in cancer. Nat Rev Genet 3, 415-428 (2002). https://doi.org:10.1038/nrg816
51 Vogelstein, B. et al. Cancer genome landscapes. Science 339, 1546-1558 (2013). https://doi.org/10.1126/science.1235122
52 De Carvalho, D. D. et al. DNA methylation screening identifies driver epigenetic events of cancer cell survival. Cancer Cell 21, 655-667 (2012). https://doi.org:10.1016/j.ccr.2012.03.045
53 Jones, P. A., Issa, J. P. & Baylin, S. Targeting the cancer epigenome for therapy. Nat Rev Genet 17, 630-641 (2016). https://doi.org:10.1038/nrg.2016.93
54 Mendoza, P. R. & Grossniklaus, H. E. The Biology of Retinoblastoma. Prog Mol Biol Transl Sci 134, 503-516 (2015). https://doi.org:10.1016/bs.pmbts.2015.06.012
55 Dyson, N. J. RB1: a prototype tumor suppressor and an enigma. Genes Dev 30, 1492-1502 (2016). https://doi.org:10.1101/gad.282145.116
56 Port, J. et al. Colorectal Tumors Require NUAK1 for Protection from Oxidative Stress. Cancer Discov 8, 632-647 (2018). https://doi.org:10.1158/2159-8290.CD-17-0533
57 Si, J. et al. Hematopoietic Progenitor Kinase1 (HPK1) Mediates T Cell Dysfunction and Is a Druggable Target for T Cell-Based Immunotherapies. Cancer Cell 38, 551-566 e511 (2020). https://doi.org:10.1016/j.ccell.2020.08.001
58 Laird, P. W. Principles and challenges of genomewide DNA methylation analysis. Nat Rev Genet 11, 191-203 (2010). https://doi.org:10.1038/nrg2732
59 Kim, M. E. et al. Aqueous Humor as a Liquid Biopsy for Retinoblastoma: Clear Corneal Paracentesis and Genomic Analysis. J Vis Exp (2021). https://doi.org:10.3791/62939
60 Singh, H. P. et al. Developmental stage-specific proliferation and retinoblastoma genesis in RB-deficient human but not mouse cone precursors. Proc Natl Acad Sci U SA 115, E9391-E9400 (2018). https://doi.org:10.1073/pnas.1808903115
61 Campan, M., Weisenberger, D. J., Trinh, B. & Laird, P. W. MethyLight. Methods Mol Biol 507, 325-337 (2009). https://doi.org:10.1007/978-1-59745-522-0_23
62 Moran, S., Arribas, C. & Esteller, M. Validation of a DNA methylation microarray for 850,000 CpG sites of the human genome enriched in enhancer sequences. Epigenomics 8, 389-399 (2016). https://doi.org:10.2217/epi.15.114
63 Krainer, J. et al. EPIC-TABSAT: analysis tool for targeted bisulfite sequencing experiments and array-based methylation studies. Nucleic Acids Res 47, W166-W170 (2019). https://doi.org:10.1093/nar/gkz398
64 Aran, D., Sirota, M. & Butte, A. J. Systematic pan-cancer analysis of tumour purity. Nat Commun 6, 8971 (2015). https://doi.org:10.1038/ncomms9971
65 Horvath, S. DNA methylation age of human tissues and cell types. Genome Biol 14, R115 (2013). https://doi.org:10.1186/gb-2013-14-10-r115
66 Gu, Z., Eils, R. & Schlesner, M. Complex heatmaps reveal patterns and correlations in multidimensional genomic data. Bioinformatics 32, 2847-2849 (2016). https://doi.org:10.1093/bioinformatics/btw313
67 Robinson, M. D., McCarthy, D. J. & Smyth, G. K. edgeR: a Bioconductor package for differential expression analysis of digital gene expression data. Bioinformatics 26, 139-140 (2010). https://doi.org:10.1093/bioinformatics/btp616
68 Kupershmidt, I. et al. Ontology-based meta-analysis of global collections of high760 throughput public data. PLOS One 5 (2010). https://doi.org:10.1371/journal.pone.0013066
69 Zhou, W., Triche, T. J., Jr., Laird, P. W. & Shen, H. SeSAMe: reducing artifactual detection of DNA methylation by Infinium BeadChips in genomic deletions. Nucleic Acids Res 46, e123 (2018). https://doi.org:10.1093/nar/gky691
70 Li, H.-T. Source Code for “Characterizing DNA methylation signatures of retinoblastoma using the aqueous humor liquid biopsy.”. Github (2022). https://doi.org:10.5281/zenodo.7005924

Various embodiments of the invention are described herein. While these descriptions directly describe embodiments, it is understood that those skilled in the art may conceive modifications and/or variations to the specific embodiments shown and described herein. Any such modifications or variations that fall within the purview of this description are intended to be included therein as well. Unless specifically noted, it is the intention of the inventors that the words and phrases in the specification and claims be given the ordinary and accustomed meanings to those of ordinary skill in the applicable art(s).

The description of various embodiments of the invention known to the applicant at this time of filing the application has been presented and is intended for the purposes of illustration and description. The present description is not intended to be exhaustive nor limit the invention to the precise form disclosed and many modifications and variations are possible in the light of the above teachings. The embodiments described serve to explain the principles of the invention and its practical application and to enable others skilled in the art to utilize the invention in various embodiments and with various modifications as are suited to the particular use contemplated. Therefore, it is intended that the invention not be limited to the particular embodiments disclosed for carrying out the invention.

While particular embodiments of the present invention have been shown and described, it will be obvious to those skilled in the art that, based upon the teachings herein, changes and modifications may be made without departing from this invention and its broader aspects and, therefore, the appended claims are to encompass within their scope all such changes and modifications as are within the true spirit and scope of this invention. It will be understood by those within the art that, in general, terms used herein are generally intended as “open” terms (e.g., the term “including” should be interpreted as “including but not limited to,” the term “having” should be interpreted as “having at least,” the term “includes” should be interpreted as “includes but is not limited to,” etc.).

All publications herein are incorporated by reference to the same extent as if each individual publication or patent application was specifically and individually indicated to be incorporated by reference. The following description includes information that may be useful in understanding the present invention. It is not an admission that any of the information provided herein is prior art or relevant to the presently claimed invention, or that any publication specifically or implicitly referenced is prior art.

1. A method to diagnose retinoblastoma (RB) in a subject comprising providing an aqueous humor (AH) sample from said subject;
measuring methylation levels of cell-free DNA (cfDNA) from said AH sample; and
determining likelihood of RB if at least one gene in said AH sample of cfDNA is hypermethylated or hypomethylated compared to that of a control.

2.-3. (canceled)

4. The method of claim 1, wherein the control is AH from an unaffected subject or normal control sample.

5. The method of claim 1, wherein the methylation level of a promoter region of at least one gene is measured.

6. The method of claim 1, wherein the methylation level of a gene body of the at least one gene is measured.

7. The method of claim 1, wherein the at least one gene is one provided in FIG. 10.

8. The method of claim 1, wherein the methylation of at least 2, 3, 4, 5, 6, 7, 8, 9, 10 or more genes is determined.

9. The method of claim 1, wherein methylation of status of all genes provided in FIG. 10 is determined.

10. The method of claim 1, further comprising treating said subject for RB.

11. The method of claim 10 wherein the treatment is cryotherapy, thermotherapy, chemotherapy, radiation therapy, internal radiation therapy, high-dose chemotherapy with stem cell rescue, surgery, targeted therapy, those therapies listed in FIG. 10 or a combination thereof.

12. The method of claim 7, further comprising treating said subject, wherein the treatment is correlated with the altered methylation state of the gene as provided in FIG. 10.

13. A method to predict severity of retinoblastoma (RB) in a subject comprising
providing an aqueous humor (AH) sample from said subject;
detecting methylation of cell-free DNA (cfDNA) from said AH sample; and
determining severity of RB if a plurality of genes in said AH sample is hypermethylated or hypomethylated compared to that of a control.

14.-15. (canceled)

16. The method of claim 13, wherein the control is AH from a subject without RB.

17. The method of claim 13, wherein the methylation of at least 2, 3, 4, 5, 6, 7, 8, 9, 10 or more genes is determined.

18. The method of claim 13, wherein the plurality of genes is selected from the genes listed in FIG. 8.

19. The method of claim 18, wherein the severe or more aggressive RB is predicted based on altered methylation status of the plurality of genes identified in FIG. 8 by Type I or Type II probes that show altered methylation status of genes that identified with Cluster B of FIG. 7.

20. The method of claim 13, further comprising treating said subject for RB.

21. The method of claim 20, wherein the treatment is cryotherapy, thermotherapy, chemotherapy, radiation therapy, internal radiation therapy, high-dose chemotherapy with stem cell rescue, surgery, targeted therapy, or a combination thereof.

22. The method of claim 19, further comprising treating said subject for RB, wherein the treatment is enucleation.

23. The method of claim 1, wherein the DNA methylation levels are determined by sodium bisulfite pyrosequencing, methylation-sensitive single nucleotide primer extension (Ms-SNuPE) reaction, methylation-specific PCR or microarray analysis.

24. The method of claim 23, wherein the DNA methylation levels are measured after sodium bisulfite modification and analyzed using microarray analysis.

25. A method to monitor the progress of a patient on treatment for retinoblastoma (RB) comprising:
a) measuring DNA methylation levels of a panel of biomarker loci of cell free DNA (cfDNA) in an aqueous humor (AH) sample from said patient for at least two time points during a course of treatment, and
b) identifying a change in DNA methylation levels of the from the panel in a),
wherein the change in the methylation status identified in b) towards normal levels indicates the treatment is therapeutically efficacious for the patient,
wherein normal levels are determined by comparison to a suitable control sample.

26. The method of claim 25, wherein the change in methylation levels is in one or more genes provided in FIG. 10.

27.-36. (canceled)


SEQUENCE LISTING (8 sequences)

SEQ ID NO: 1 - DNA, 23 bp, synthetic construct
  source 1..23: mol_type = other DNA
gggaaagagggattttttgaatt

SEQ ID NO: 2 - DNA, 29 bp, synthetic construct
  source 1..29: mol_type = other DNA
aactaccaaaactaactataaccccacaa

SEQ ID NO: 3 - DNA, 28 bp, synthetic construct
  source 1..28: mol_type = other DNA
atttatttaagtgttaattaggttgggt

SEQ ID NO: 4 - DNA, 30 bp, synthetic construct
  source 1..30: mol_type = other DNA
aatttaaaatcataacttaccaaaactcaa

SEQ ID NO: 5 - DNA, 22 bp, synthetic construct
  source 1..22: mol_type = other DNA
gggatttgagggatagtgattt

SEQ ID NO: 6 - DNA, 25 bp, synthetic construct
  source 1..25: mol_type = other DNA
caaaattcaaatttcaaccccctaa

SEQ ID NO: 7 - DNA, 25 bp, synthetic construct
  source 1..25: mol_type = other DNA
agtattgggagttatttttgaggtt

SEQ ID NO: 8 - DNA, 22 bp, synthetic construct
  source 1..22: mol_type = other DNA
caatcctctcctaaaaaccaaa